(19) United States
(12) Patent Application Publication
(10) Pub. No.: US 2025/0353827 A1
(43) Pub. Date: Nov. 20, 2025

(54) INDOLINE DERIVATIVES AS SEROTONERGIC AGENTS USEFUL FOR THE TREATMENT OF DISORDERS RELATED THERETO

(71) Applicant: MINDSET PHARMA INC., Toronto (CA)

(72) Inventors: Abdelmalik SLASSI, Mississauga (CA); Joseph A. ARAUJO, Grimsby (CA)

(21) Appl. No.: 18/857,230

(22) PCT Filed: Apr. 18, 2023
(86) PCT No.: PCT/CA2023/050521

§ 371 Date: Oct. 16, 2024

Related U.S. Application Data
(60) Provisional application No. 63/332,450, filed on Apr. 19, 2022.

(51) Int. Cl. C07D 401/04; A61K 31/4045; A61K 31/407; A61K 31/422; A61K 31/427; A61K 31/439; C07B 59/00; C07D 209/16; C07D 403/04; C07D 409/04; C07D 413/04; C07D 417/04; C07D 491/056

(52) U.S. Cl. CPC C07D 401/04; A61K 31/4045; A61K 31/407; A61K 31/422; A61K 31/427; A61K 31/439; C07B 59/002; C07D 209/16; C07D 403/04; C07D 409/04; C07D 413/04; C07D 417/04; C07D 491/056

(57) ABSTRACT

The present application relates to indoline derivatives of general Formula (I), to processes for their preparation, to compositions comprising them and to their use in activation of a serotonin receptors in a cell, as well as to treating diseases, disorders or conditions by activation of a serotonin receptors in a cell. The diseases, disorders or conditions include, for example, psychosis, mental illnesses and CNS disorders and/or associated endophenotypes and/or symptom clusters. Wherein Q is (Q3).

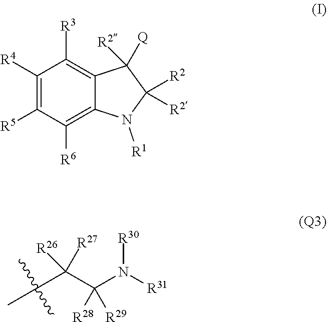

### RELATED APPLICATIONS

The present application claims the benefit of priority of co-pending U.S. provisional patent application No. 63/332,450 filed on Apr. 19, 2022, the contents of which are incorporated herein by reference in their entirety.

### FIELD

The application relates to indoline compounds for the treatment of different conditions that are treated by activation of serotonin receptors, for example, mental illnesses and neurological disease, in the fields of psychiatry, neurobiology and pharmacotherapy.

### BACKGROUND OF THE APPLICATION

The present applicant has several co-pending applications disclosing novel indole derivatives as serotonergic psychedelic agents for the treatment of various CNS disorders, including PCT patent application publication nos. WO2021/155470, WO2021/155468 and WO2021/155467, and PCT application S.N. PCT/CA2022/-50295 and U.S. provisional application Ser. No. 63/260,470 and U.S. provisional application Ser. No. 63/449,715. These applications describe the various pharmaceutical applications for such agents, including in the treatment of mental illnesses and neurological disease, and use in the fields of psychiatry, neurobiology and pharmacotherapy.

Psychedelics show promise in treating diseases such as major depressive disorder, treatment resistant depression, post-traumatic stress disorder (PTSD), and substance use disorder, in addition to end of life existential distress and others. To date, hundreds of millions of dollars have been invested in companies developing new psychedelic drugs in (see for example, C&EN Mar. 7, 2022). Psychedelics are therefore destined to enter the mainstream of modern medicine as treatments for intractable mental illness.

The therapeutic effectiveness of classic psychedelics such as mescaline, N,N-dimethyltryptamine (DMT), psilocybin and lysergic acid diethylamide (LSD) are largely believed to be mediated by the activation of serotonin 2A (5-HT_{2A}) receptors. These drugs are increasingly being evaluated and used to treat neuropsychiatric diseases.

Many psychedelics have side effects, for example, hallucinogenic effects, impeding their use outside of a clinical setting.

Some potential non-hallucinogenic psychedelic analogs have been reported (Cunningham M. J. et al., ACS Chem. Neurosci. 2023, 14, 1, 119-135, Hofman, A. LSD, My Problem Child: Reflections on Sacred Drugs, Mysticism, and Science; MAPS, Multidisciplinary Association for Psychedelic Studies: Santa Cruz, CA, 2009, Weingartner, H. et al., J. Clin. Pharmacol. New Drugs 1971, 11, 103-111, Shulgin. A. T. 2-Amino-1-(2,5-dimethoxyphenyl) butanes U.S. Pat. No. 4,105,695A, Shulgin, A. T. Soc. Pharmacol. 1987, 1, 279-290, Cameron, et al. Nature 2021, 589(7842) 474-479, Lu J. et al., Molecular Psychiatry, 2021, 26, 6237-6252, Cao et al., Science 2022, 375, 403-411, Vargas et al., Science, 2023, 379, 700-706). While results with these non-hallucinogenic 5-HT_{2A} agonists have been encouraging, non-hallucinogenic 5-HT_{2A} agonist therapeutic development remains in its infancy.

There remains a need to provide new psychedelic and non-hallucinogenic 5-HT_{2A} agonist molecules for the treatment, for example, of mental diseases.

### SUMMARY OF THE APPLICATION

Compounds of the present application modulate the activity of serotonin receptor subtypes, in particular 5-HT_{2A}, by direct binding to these receptors.

Accordingly, the present application includes compounds of Formula I:

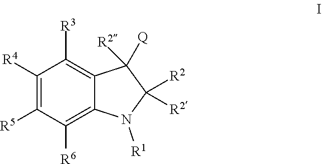

or a pharmaceutically acceptable salt, solvate and/or prodrug thereof,
wherein:
R¹ is selected from C(O)R⁷, CO₂R⁷, C(O)N(R⁷)(R⁷), S(O)R⁷, SO₂R⁷, C₁₋₄alkyleneR⁷ and R⁷;
Q is Q3:

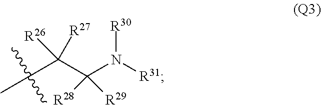

[symbol] over a bond means that the bond is attached to a remaining portion of the compound;
R², R^{2′} and R^{2″} are independently selected from H, halo, C₁₋₆alkyl, C₁₋₆alkoxy C₁₋₆ alkyleneN(R^{2a})(R^{2b}) and SC₁₋₆alkyl;
R^{2a} and R^{2b} are independently selected from H and C₁₋₆alkyl;
R³, R⁴, R⁵ and R⁶ are independently selected from H, halo, N(R^{5′})(R^{6′}), C₁₋₆alkyl, C₁₋₆alkoxy, C₁₋₆alkyleneN(R^{5′})(R^{6′}) and SC₁₋₆alkyl, the latter four groups being optionally substituted with one or two substituents selected from OH and C₁₋₆alkoxy, or
two adjacent of R³, R⁴, R⁵ and R⁶ are linked together to form O—(CH₂)₁₋₂₀ and the remaining of R³, R⁴, R⁵ and R⁶ are independently selected from H, halo, C₁₋₆alkyl, C₁₋₆alkoxy, N(R^{5′})(R^{6′), C}₁₋₆alkyleneN(R^{5′})(R^{6′}) and SC₁₋₆alkyl, or
one of R³, R⁴, R⁵ and R⁶ is selected from A, O-A and C₁₋₄alkyleneA and the other of R⁴ and remaining of R³, R⁴, R⁵ and R⁶ are, independently H or halo;
R^{5′} and R^{6′} are independently selected from H and C₁₋₆alkyl;
A is selected from phenyl, C₃₋₆cycloalkyl, 3- to 6-membered heterocycloalkyl comprising 1 to 4 heteromoeities independently selected from O, S, S(O), SO₂, N and NR⁵⁴ and 5- to 6-membered heteroaryl comprising 1 to 4 heteromoeities independently selected from O, S, S(O), SO₂, N and NR⁵⁴, wherein the phenyl, C₃₋₁₀cycloalkyl, 3- to 6-membered heterocycloalkyl and 5- to 6-membered heteroaryl are optionally substituted with one or more substituents independently selected from halo, C₁₋₄alkyl, OC₁₋₄alkyl, N(R^{54a})(R^{54b}), C₁₋₄alkyleneN(R^{54a})(R^{54b}) and SC₁₋₄alkyl;
R⁷ is selected from H, C₁₋₆alkyl, C₂₋₆alkenyl, C₂₋₆alkynyl, phenyl, C₃₋₆cycloalkyl, 3- to 6-membered heterocycloalkyl comprising 1 to 4 heteromoeities independently selected from O, S, N and NR^{7″}, and 5- to 6-membered heteroaryl comprising 1 to 4 heteromoeities independently selected from O, S, N and NR^{7″}, wherein the latter 7 groups are optionally substituted with one or more substituents independently selected from halo, OR⁵⁵, N(R⁵⁵)(R⁵⁶) and SR⁵⁵ and/or the C₁₋₆alkyl is optionally interrupted by one to three heteromoieties independently selected from O, C(O), CO₂ and NR⁵⁷;
R^{7′} is selected from H and C₁₋₆alkyl;
R^{7″} is selected from H and C₁₋₆alkyl;
R²⁶, R²⁷, R²⁸ and R²⁹ are independently selected from H, halo and C₁₋₆alkyl;
R³⁰ and R³¹ are independently selected from H, C₁₋₆alkyl and C(O)C₁₋₆alkyl, or
R³⁰ and R³¹, together with the N atom to which they are bound, form a 3- to 6-membered heterocyclic ring which optionally comprises one or two additional heteromoieties independently selected from O, S, S(O), SO₂, N, and NR⁵⁸;
R⁵⁴, R⁵⁵, R⁵⁶, R⁵⁷ and R⁵⁸ are independently selected from H and C₁₋₆alkyl;
R^{54a} and R⁵⁴¹ are independently selected from H and C₁₋₆alkyl; and
all available hydrogen atoms are optionally and independently substituted with a fluorine atom or chlorine atom and all available atoms are optionally substituted with alternate isotope thereof,
provided that when R²⁶, R²⁷, R²⁸ and R²⁹ are all H and R³⁰ and R³¹ are H or CH₃, and:
R¹ is R⁷ and R⁷ is H,
R², R^{2′}, R^{2″}, R³, R⁴, R⁵ and R⁶ are all H,
R¹, R², R^{2′}, R^{2″}, R³, R⁵ and R⁶ are all H and R⁴ is OCH₃, or
R¹, R², R^{2′}, R^{2″}, R³, R⁴, R⁵ and R⁶ are all H and R⁵ is OCH₃,
then the compound of Formula I is an (R)- or (S)-enantiomer of the carbon to which Q is attached.

In some embodiments, the compound of Formula I is defined as follows:

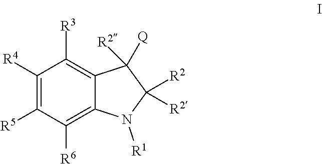

or a pharmaceutically acceptable salt, solvate and/or prodrug thereof,
R¹ is H;
Q is Q3:

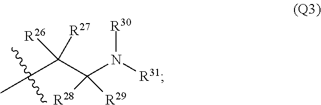

[symbol] over a bond means that the bond is attached to a remaining portion of the compound;
R², R^{2′} and R^{2″} are independently selected H, D and F;
R³, R⁴, R⁵ and R⁶ are independently selected from H, C₁₋₄alkoxy, C₁₋₄fluoralkoxy and C₁₋₄deuteroalkoxy, or
two adjacent of R³, R⁴, R⁵ and R⁶ are linked together to form O—(CH₂)₁₋₂O and the remaining of R³, R⁴, R⁵ and R⁶ are independently selected from H, halo, C₁₋₆alkyl and C₁₋₆alkoxy;
R²⁶, R²⁷, R²⁸ and R²⁹ are independently selected from H, halo and C₁₋₆alkyl;
R³⁰ and R³¹ are independently selected from H, C₁₋₆alkyl and C(O)C₁₋₆alkyl, or
R³⁰ and R³¹, together with the N atom to which they are bound, form a 3- to 6-membered heterocyclic ring which optionally comprises one or two additional heteromoieties independently selected from O, S, S(O), SO₂, N, and NR⁵⁸;
R⁵⁸ is selected from H and C₁₋₆alkyl; and
all available hydrogen atoms are optionally and independently substituted with a fluorine atom or chlorine atom and all available atoms are optionally substituted with alternate isotope thereof,
provided that when R²⁶, R²⁷, R²⁸ and R²⁹ are all H and R³⁰ and R³¹ are H or CH₃, and:
R¹, R², R^{2′}, R^{2″}, R³, R⁴, R⁵ and R⁶ are all H,
R¹, R², R^{2′}, R^{2″}, R³, R⁵ and R⁶ are all H and R⁴ is OCH₃, or
R¹, R², R^{2′}, R^{2″}, R³, R⁴, R⁵ and R⁶ are all H and R⁵ is OCH₃,
then the compound of Formula I is an (R)- or (S)-enantiomer of the carbon to which Q is attached.

In a further embodiment, the compounds of the application are used as medicaments. Accordingly, the application also includes a compound of the application for use as a medicament.

The present application also includes a method of treating psychosis or psychotic symptoms comprising administering a therapeutically effective amount of one or more compounds of the application to a subject in need thereof.

The present application also includes a method of treating a mental illness comprising administering a therapeutically effective amount of one or more compounds of the application to a subject in need thereof.

The present application also includes a method of treating a central nervous system (CNS) disease, disorder or condition and/or a neurological disease, disorder or condition.

The present application also includes a method of treating an endophenotype and/or symptom cluster associated with a disease, disorder or condition that is treated by activation of a serotonin receptor.

The present application also includes methods and uses of a non-hallucinogenic compound of Formula II

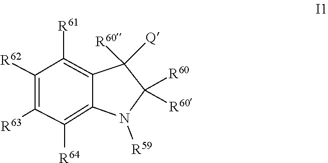

or a pharmaceutically acceptable salt, solvate and/or prodrug thereof,
wherein:
R⁵⁹ is selected from C(O)R⁶⁵, CO₂R⁶⁵, C(O)N(R⁶⁵)(′^{65′}), S(O)R⁶⁵, SO₂R⁶⁵, C₁₋₆alkyleneR⁶⁵ and R⁶⁵;
Q′ is Q3′:

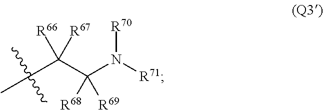

[symbol] over a bond means that the bond is attached to a remaining portion of the compound;
R⁶⁰, R^{60′} and R^{60″} are independently selected from H, halo, C₁₋₆alkyl and C₁₋₆alkoxy, C₁₋₁₀ alkyleneN(R^{60a})(R^{60b}) and SC₁₋₆alkyl;
R^{60a} and R^{60b} are independently selected from H and C₁₋₆alkyl;
R⁶¹, R⁶², R⁶³ and R⁶⁴ are independently selected from H, halo, N(R^{63′})(R^{64′}), C₁₋₆alkyl, C₁₋₆ alkoxy, C₁₋₆alkyleneN(R^{63′})(R^{64′}) and SC₁₋₆alkyl, the latter four groups being optionally substituted with one or two substituents selected from OH and C₁₋₆alkoxy, or
two adjacent of R⁶¹, R⁶², R⁶³ and R⁶⁴ are linked together to form O—(CH₂)₁₋₂O and the remaining of R⁶¹, R⁶², R⁶³ and R⁶⁴ are independently selected from H, halo, C₁₋₆alkyl and C₁₋₆alkoxy, N(R^{63′})(R^{64′}), C₁₋₆alkyleneN(R^{63′})(R^{64′}) and SC₁₋₆alkyl; or
one of R⁶¹, R⁶², R⁶³ and R⁶⁴ is selected from A′, O-A′ and C₁₋₄alkyleneA′ and the remaining of R⁶¹, R⁶², R⁶³ and R⁶⁴ are independently H or halo;
R^{63′} and R^{64′} are independently selected from H and C₁₋₆alkyl;
A′ is selected from phenyl, C₃₋₆cycloalkyl, 3- to 6-membered heterocycloalkyl comprising 1 to 4 heteromoeities independently selected from O, S, S(O), SO₂, N and NR⁷² and 5- to 6-membered heteroaryl comprising 1 to 4 heteromoeities independently selected from O, S, S(O), SO₂, N and NR⁷², wherein the phenyl, C₃₋₁₀cycloalkyl, 3- to 6-membered heterocycloalkyl and 5- to 6-membered heteroaryl are optionally substituted with one or more substituents independently selected from halo, C₁₋₄alkyl, OC₁₋₄alkyl, N(R^{72a})(R^{72b}), C₁₋₆alkyleneN(R^{72a})(R^{72b}) and SC₁₋₆alkyl;
R⁶⁵ is selected from H, C₁₋₆alkyl, C₂₋₆alkenyl, C₂₋₆alkynyl, phenyl, C₃₋₆cycloalkyl, 3- to 6-membered heterocycloalkyl comprising 1 to 4 heteromoeities independently selected from O, S, N and NR^{65″} and 5- to 6-membered heteroaryl comprising 1 to 4 heteromoeities independently selected from O, S, N and NR^{65″}, wherein the latter 5 groups are optionally substituted with one or more substituents independently selected from halo, OR⁷³, N(R⁷³)(R⁷⁴) and SR⁷³ and/or the C₁₋₆alkyl is optionally interrupted by one to three heteromoieties independently selected from O, C(O), CO₂ and NR⁷⁵;
R^{65′} is selected from H and C₁₋₆alkyl;
R^{65″} is selected from H and C₁₋₆alkyl;
R⁶⁶, R⁶⁷, R⁶⁸ and R⁶⁹ are independently selected from H, halo and C₁₋₆alkyl;
R⁷⁰ and R⁷¹ are independently selected from H, C₁₋₆alkyl and C(O)C₁₋₆alkyl, or
R⁷⁰ and R⁷¹, together with the N atom to which they are bound, form a 3- to 6-membered heterocyclic ring which optionally comprises one or two additional heteromoieties independently selected from O, S, S(O), SO₂, N, and NR⁷⁶;
R⁷², R⁷³, R⁷⁴, R⁷⁵ and R⁷⁶ are independently selected from H and C₁₋₆alkyl;
R^{72a} and R^{72b} are independently selected from H and C₁₋₆alkyl; and
all available hydrogen atoms are optionally and independently substituted with a fluorine atom or chlorine atom and all available atoms are optionally substituted with alternate isotope thereof.

The application additionally provides a process for the preparation of compounds of the application. General and specific processes are discussed in more detail below and set forth in the examples below.

The application also provides a process for the preparation of compounds of Formula II, or a pharmaceutically acceptable salt, solvate and/or prodrug thereof. General and specific processes are discussed in more detail below and set forth in the examples below.

Other features and advantages of the present application will become apparent from the following detailed description. It should be understood, however, that the detailed description and the specific examples, while indicating embodiments of the application, are given by way of illustration only and the scope of the claims should not be limited by these embodiments but should be given the broadest interpretation consistent with the description as a whole.

### BRIEF DESCRIPTION OF THE DRAWINGS

The present application will be described in greater detail with reference to the attached drawings in which:

FIG. 1 shows the scores for exemplary compound II-1 in the mouse head twitch assays at doses of up to 30 mg/kg.

### DETAILED DESCRIPTION

### I. Definitions

Unless otherwise indicated, the definitions and embodiments described in this and other sections are intended to be applicable to all embodiments and aspects of the present application herein described for which they are suitable as would be understood by a person skilled in the art.

The term “compound(s) of the application” or “compound(s) of the present application” and the like as used herein refers to a compound of Formula I and includes pharmaceutically acceptable salts, solvates and/or prodrugs thereof as well as all stereoisomers and regioisomers.

The term “composition(s) of the application” or “composition(s) of the present application” and the like as used herein refers to a composition, such a pharmaceutical composition, comprising one or more compounds of the application.

The term “and/or” as used herein means that the listed items are present, or used, individually or in combination. In effect, this term means that “at least one of or “one or more” of the listed items is used or present. The term “and/or” with respect to pharmaceutically acceptable salts and/or solvates thereof means that the compounds of the application exist as individual salts and solvates, as well as a combination of, for example, a salt of a solvate of a compound of the application.

As used in the present application, the singular forms “a”, “an” and “the” include plural references unless the content clearly dictates otherwise. For example, an embodiment including “a compound” should be understood to present certain aspects with one compound, or two or more additional compounds.

As used in this application and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

The term “consisting” and its derivatives as used herein are intended to be closed terms that specify the presence of the stated features, elements, components, groups, integers and/or steps and also exclude the presence of other unstated features, elements, components, groups, integers and/or steps.

The term “consisting essentially of”, as used herein, is intended to specify the presence of the stated features, elements, components, groups, integers and/or steps as well as those that do not materially affect the basic and novel characteristic(s) of these features, elements, components, groups, integers and/or steps.

In embodiments comprising an “additional” or “second” component, such as an additional or second compound, the second component as used herein is chemically different from the other components or first component. A “third” component is different from the other, first and second components and further enumerated or “additional” components are similarly different.

The term “suitable” as used herein means that the selection of the particular compound or conditions would depend on the specific synthetic manipulation to be performed, the identity of the molecule(s) to be transformed and/or the specific use for the compound, but the selection would be well within the skill of a person trained in the art. All process/method steps described herein are to be conducted under conditions sufficient to provide the product shown. A person skilled in the art would understand that all reaction conditions, including, for example, reaction solvent, reaction time, reaction temperature, reaction pressure, reactant ratio and whether or not the reaction should be performed under an anhydrous or inert atmosphere, can be varied to optimize the yield of the desired product and it is within their skill to do so.

The terms “about”, “substantially” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies or unless the context suggests otherwise to a person skilled in the art.

The present description refers to a number of chemical terms and abbreviations used by those skilled in the art. Nevertheless, definitions of selected terms are provided for clarity and consistency.

The term “solvate” as used herein means a compound, or a salt and/or prodrug of a compound, wherein molecules of a suitable solvent are incorporated in the crystal lattice.

The term “prodrug” as used herein means a compound, or salt of a compound, that, after administration, is converted into an active drug.

The term “zwitterion” as used herein means a molecule that contains an equal number of positively- and negatively-charged functional groups. Zwitterions are also known as “inner salts” and “dipolar ions”.

The term “alkyl” as used herein, whether it is used alone or as part of another group, means straight or branched chain, saturated alkyl groups. The number of carbon atoms that are possible in the referenced alkyl group are indicated by the prefix “Cₙ₁₋ₙ₂”. Thus, for example, the term “C₁₋₆alkyl” (or “C₁-C₆alkyl”) means an alkyl group having 1, 2, 3, 4, 5, or carbon atoms.

The term “alkenyl” as used herein, whether it is used alone or as part of another group, means straight or branched chain, unsaturated alkyl groups containing at least one double bond. The number of carbon atoms that are possible in the referenced alkylene group are indicated by the prefix “Cₙ₁₋ₙ₂”. For example, the term C₂₋₆alkenyl means an alkenyl group having 2, 3, 4, 5 or 6 carbon atoms and at least one double bond.

The term “alkynyl” as used herein, whether it is used alone or as part of another group, means straight or branched chain, unsaturated alkynyl groups containing at least one triple bond. The number of carbon atoms that are possible in the referenced alkyl group are indicated by the prefix “Cₙ₁₋ₙ₂”. For example, the term C₂₋₆alkynyl means an alkynyl group having 2, 3, 4, 5 or 6 carbon atoms.

As used herein, the term “alkoxy” as used herein, alone or in combination, includes an alkyl group connected to an oxygen-connecting atom.

The term “fluoroalkoxy” as used herein refers to an alkoxy group as defined above in which one or more of the available hydrogen atoms have been replaced with a fluorine.

The term “deuteroalkoxy” as used herein refers to an alkoxy group as defined above in which one or more of the available hydrogen atoms have been replaced with a deuterium.

The term “cycloalkyl,” as used herein, whether it is used alone or as part of another group, means a saturated carbocyclic group containing from 3 to 6 carbon atoms and one or more rings. The number of carbon atoms that are possible in the referenced cycloalkyl group are indicated by the numerical prefix “Cₙ₁₋ₙ₂”. For example, the term C₃₋₁₀cycloalkyl means a cycloalkyl group having 3, 4, 5 or 6 carbon atoms.

The term “heterocycloalkyl” as used herein, whether it is used alone or as part of another group, refers to cyclic groups containing at least one non-aromatic ring containing from 3 to 6 atoms in which one or more of the atoms are a heteromoiety selected from O, S, S(O), SO₂ and N and the remaining atoms are C. Heterocycloalkyl groups are either saturated or unsaturated (i.e. contain one or more double bonds). When a heterocycloalkyl group contains the prefix Cₙ₁₋ₙ₂ or “n1 to n2” this prefix indicates the number of carbon atoms in the corresponding carbocyclic group, in which one or more, suitably 1 to 4, of the ring atoms is replaced with a heteromoeity as selected from O, S, S(O), SO₂ and N and the remaining atoms are C.

The term “heteroaryl” as used herein, whether it is used alone or as part of another group, refers to cyclic groups containing at least one heteroaromatic ring containing 5-6 atoms in which one or more of the atoms are a heteroatom selected from O, S and N and the remaining atoms are C. When a heteroaryl group contains the prefix Cₙ₁₋ₙ₂ this prefix indicates the number of carbon atoms in the corresponding carbocyclic group, in which one or more, suitably 1 to 4, of the ring atoms is replaced with a heteroatom as defined above.

The term “halogen” (or “halo”) whether it is used alone or as part of another group, refers to a halogen atom and includes fluoro, chloro, bromo and iodo.

The term “haloalkyl” as used herein refers to an alkyl group as defined above in which one or more of the available hydrogen atoms have been replaced with a halogen. Thus, for example, “C₁₋₆haloalkyl” refers to a C₁ to C₆ linear or branched alkyl group as defined above with one or more halogen substituents.

The term “deuteroalkyl” as used herein refers to an alkyl group as defined above in which one or more of the available hydrogen atoms have been replaced with a deuterium.

Thus, for example, “C₁₋₆deuteroalkyl” refers to a C₁ to C₆ linear or branched alkyl group as defined above with one or more deuterium substituents.

The term “available”, as in “available hydrogen atoms” or “available atoms” refers to atoms that would be known to a person skilled in the art to be capable of replacement by a substituent.

As used herein, the term “one or more” item includes a single item selected from the list as well as mixtures of two or more items selected from the list.

The term “alternate isotope thereof” as used herein refers to an isotope of an element that is other than the isotope that is most abundant in nature.

The term “all available atoms are optionally replaced with alternate isotope” as used herein means that available atoms are optionally replaced with an isotope of that atom having the same atomic number, but an atomic mass or mass number different from the atomic mass or mass number predominantly found in nature.

In the compounds of general Formula I and pharmaceutically acceptable salts and/or solvates thereof, the atoms may exhibit their natural isotopic abundances, or one or more of the atoms may be artificially enriched in a particular isotope having the same atomic number, but an atomic mass or mass number different from the atomic mass or mass number predominantly found in nature. The present disclosure is meant to include all suitable isotopic variations of the compounds of general Formula I and pharmaceutically acceptable salts and/or solvates thereof. For example, different isotopic forms of hydrogen (H) include protium (¹H), deuterium (²H) and tritium (³H). Protium is the predominant hydrogen isotope found in nature.

In the compounds of general Formula II, and pharmaceutically acceptable salts and/or solvates thereof, the atoms may exhibit their natural isotopic abundances, or one or more of the atoms may be artificially enriched in a particular isotope having the same atomic number, but an atomic mass or mass number different from the atomic mass or mass number predominantly found in nature. The present disclosure is meant to include all suitable isotopic variations of the compounds of general Formula II and pharmaceutically acceptable salts and/or solvates thereof. For example, different isotopic forms of hydrogen (H) include protium (¹H), deuterium (²H) and tritium (³H). Protium is the predominant hydrogen isotope found in nature.

The term “compound” refers to the compound and, in certain embodiments, to the extent they are stable, any hydrate or solvate thereof. A hydrate is the compound complexed with water and a solvate is the compound complexed with a solvent, which may be an organic solvent or an inorganic solvent. A “stable” compound is a compound that can be prepared and isolated and whose structure and properties remain or can be caused to remain essentially unchanged for a period of time sufficient to allow use of the compound for the purposes described herein (e.g., therapeutic administration to a subject). The compounds of the present application are limited to stable compounds embraced by general Formula I, or pharmaceutically acceptable salts and/or solvates thereof. The compounds of the present application are limited to stable compounds embraced by general Formula I, or pharmaceutically acceptable prodrugs thereof. Further compounds of Formula II, or pharmaceutically acceptable salts, solvates and/or prodrugs thereof, are limited to stable compounds of Formula II, or pharmaceutically acceptable salts, solvates and/or prodrugs thereof.

The term “pharmaceutically acceptable” means compatible with the treatment of subjects.

The term “pharmaceutically acceptable carrier” means a non-toxic solvent, dispersant, excipient, adjuvant or other material which is mixed with the active ingredient in order to permit the formation of a pharmaceutical composition, i.e., a dosage form capable of administration to a subject.

The term “pharmaceutically acceptable salt” means either an acid addition salt or a base addition salt which is suitable for, or compatible with, the treatment of subjects.

An acid addition salt suitable for, or compatible with, the treatment of subjects is any non-toxic organic or inorganic acid addition salt of any basic compound.

A base addition salt suitable for, or compatible with, the treatment of subjects is any non-toxic organic or inorganic base addition salt of any acidic compound.

The term “protecting group” or “PG” and the like as used herein refers to a chemical moiety which protects or masks a reactive portion of a molecule to prevent side reactions in those reactive portions of the molecule, while manipulating or reacting a different portion of the molecule. After the manipulation or reaction is complete, the protecting group is removed under conditions that do not degrade or decompose the remaining portions of the molecule. The selection of a suitable protecting group can be made by a person skilled in the art. Many conventional protecting groups are known in the art, for example as described in “Protective Groups in Organic Chemistry” McOmie, J. F. W. Ed., Plenum Press, 1973, in Greene, T. W. and Wuts, P. G. M., “Protective Groups in Organic Synthesis”, John Wiley & Sons, 3^{rd} Edition, 1999 and in Kocienski, P. Protecting Groups, 3rd Edition, 2003, Georg Thieme Verlag (The Americas).

The term “subject” as used herein includes all members of the animal kingdom including mammals, and suitably refers to humans. Thus the methods of the present application are applicable to both human therapy and veterinary applications.

The term “treating” or “treatment” as used herein and as is well understood in the art, means an approach for obtaining beneficial or desired results, including clinical results. Beneficial or desired clinical results include, but are not limited to alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease and remission (whether partial or total), whether detectable or undetectable. “Treating” and “treatment” can also mean prolonging survival as compared to expected survival if not receiving treatment. “Treating” and “treatment” as used herein also include prophylactic treatment. For example, a subject with early cancer can be treated to prevent progression, or alternatively a subject in remission can be treated with a compound or composition of the application to prevent recurrence. Treatment methods comprise administering to a subject a therapeutically effective amount of one or more of the compounds of the application and optionally consist of a single administration, or alliteratively comprise a series of administrations.

As used herein, the term “effective amount” or “therapeutically effective amount” means an amount of one or more compounds of the application that is effective, at dosages and for periods of time necessary to achieve the desired result. For example, in the context of treating a disease, disorder or condition mediated or treated by agonism or activation of serotonergic receptors and downstream second messengers, an effective amount is an amount that, for example, increases said activation compared to the activation without administration of the one or more compounds.

“Palliating” a disease, disorder or condition means that the extent and/or undesirable clinical manifestations of a disease, disorder or condition are lessened and/or time course of the progression is slowed or lengthened, as compared to not treating the disorder.

The term “administered” as used herein means administration of a therapeutically effective amount of one or more compounds or compositions of the application to a cell, tissue, organ or subject.

The term “prevention” or “prophylaxis”, or synonym thereto, as used herein refers to a reduction in the risk or probability of a patient becoming afflicted with a disease, disorder or condition or manifesting a symptom associated with a disease, disorder or condition.

The “disease, disorder or condition” as used herein refers to a disease, disorder or condition treated or treatable by activation a serotonin receptor, for example 5-HT_{2A} and particularly using a serotonin receptor agonist, such as one or more compounds of the application herein described.

The term “treating a disease, disorder or condition by activation of a serotonin receptor” as used herein means that the disease, disorder or condition to be treated is affected by, modulated by and/or has some biological basis, either direct or indirect, that includes serotonergic activity, in particular increases in serotonergic activity. These diseases respond favourably when serotonergic activity associated with the disease, disorder or condition is agonized by one or more of the compounds or compositions of the application.

The term “activation” as used herein includes agonism, partial agonist and positive allosteric modulation of a serotonin receptor.

The terms “5-HT_{1A}” and “5-HT_{2A}” are used herein mean the 5-HT_{2A} and 5-HT_{2A} receptor subtypes of the 5-HT₂ serotonin receptor.

The term “therapeutic agent” as used herein refers to any drug or active agent that has a pharmacological effect when administered to a subject.

The term “equivalent dose of psilocybin” as used herein means that a compound is administered or used at a dose that is the same molar amount as a corresponding dose of psilocybin.

### II. Compounds

The present application includes compounds of Formula I:

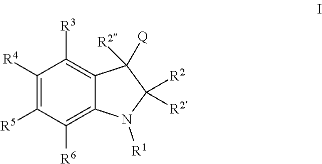

or a pharmaceutically acceptable salt, solvate and/or prodrug thereof,
wherein:
R¹ is selected from C(O)R⁷, CO₂R⁷, C(O)N(R⁷)(R^{7′}), S(O)R⁷, SO₂R⁷, C₁₋₆alkyleneR⁷ and R⁷;
Q is Q3:

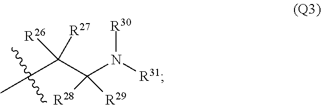

[symbol] over a bond means that the bond is attached to a remaining portion of the compound;
R², R^{2′} and R^{2″} are independently selected from H, halo, C₁₋₆alkyl, C₁₋₆alkoxy, C₁₋₆alkyleneN(R^{2a})(R^{2b}) and SC₁₋₆alkyl;
R^{2a} and R^{2b} are independently selected from H and C₁₋₆alkyl;
R³, R⁴, R⁵ and R⁶ are independently selected from H, halo, N(R^{5′})(R^{6′}), C₁₋₆alkyl, C₁₋₆alkoxy, C₁₋₆alkyleneN(R^{5′})(R^{6′}) and SC₁₋₆alkyl, the latter four groups being optionally substituted with one or two substituents selected from OH and C₁₋₆alkoxy, or
two adjacent of R³, R⁴, R⁵ and R⁶ are linked together to form O—(CH₂)₁₋₂O and the remaining of R³, R⁴, R⁵ and R⁶ are independently selected from H, halo, C₁₋₆alkyl, C₁₋₆alkoxy, N(R^{5′})(R^{6′}), C₁₋₆alkyleneN(R^{5′})(R^{6′}) and SC₁₋₆alkyl, or
one of R³, R⁴, R⁵ and R⁶ is selected from A, O-A and C₁₋₄alkyleneA and the remaining of R³, R⁴, R⁵ and R⁶ are independently H or halo;
R^{5′} and R^{6′} are independently selected from H and C₁₋₆alkyl;
A is selected from phenyl, C₃₋₆cycloalkyl, 3- to 6-membered heterocycloalkyl comprising 1 to 4 heteromoeities independently selected from O, S, S(O), SO₂, N and NR⁵⁴ and 5- to 6-membered heteroaryl comprising 1 to 4 heteromoeities independently selected from O, S, S(O), SO₂, N and NR⁵⁴, wherein the phenyl, C₃₋₁₀cycloalkyl, 3- to 6-membered heterocycloalkyl and 5- to 6-membered heteroaryl are optionally substituted with one or more substituents independently selected from halo, C₁₋₄alkyl, OC₁₋₄alkyl, N(R^{54a})(R^{54b}), C₁₋₄alkyleneN(R^{54a})(R^{54b}) and SC₁₋₄alkyl;
R⁷ is selected from H, C₁₋₆alkyl, C₂₋₆alkenyl, C₂₋₆alkynyl, phenyl, C₃₋₆cycloalkyl, 3- to 6-membered heterocycloalkyl comprising 1 to 4 heteromoeities independently selected from O, S, N and NR^{7″} and 5- to 6-membered heteroaryl comprising 1 to 4 heteromoeities independently selected from O, S, N and NR^{7″}, wherein the latter 7 groups are optionally substituted with one or more substituents independently selected from halo, OR⁵⁵, N(R⁵⁵)(R⁵⁶) and SR⁵⁵ and/or the C₁₋₆alkyl is optionally interrupted by one to three heteromoieties independently selected from O, C(O), CO₂ and NR⁵⁷;
R^{7′} is selected from H and C₁₋₆alkyl;
R^{7″} is selected from H and C₁₋₆alkyl;
R²⁶, R²⁷, R²⁸ and R²⁹ are independently selected from H, halo and C₁₋₆alkyl;
R³⁰ and R³¹ are independently selected from H, C₁₋₆alkyl and C(O)C₁₋₆alkyl, or
R³⁰ and R³¹, together with the N atom to which they are bound, form a 3- to 6-membered heterocyclic ring which optionally comprises one or two additional heteromoieties independently selected from O, S, S(O), SO₂, N, and NR⁵⁸;
R⁵⁴, R⁵⁵, R⁵⁶, R⁵⁷ and R⁵⁸ are independently selected from H and C₁₋₆alkyl; and
R^{54a} and R^{54b} are independently selected from H and C₁₋₆alkyl;
all available hydrogen atoms are optionally and independently substituted with a fluorine atom or chlorine atom and all available atoms are optionally substituted with alternate isotope thereof,
provided that when R²⁶, R²⁷, R²⁸ and R²⁹ are all H and R³⁰ and R³¹ are H or CH₃, and:
R¹ is R⁷, R⁷ is H, and R², R^{2′}, R^{2″}, R³, R⁴, R⁵ and R⁶ are all H,
R¹ is R⁷, R⁷ is H, R², R^{2′}, R^{2″}, R³, R⁵ and R⁶ are all H and R⁴ is OCH₃, or
R¹ is R⁷, R⁷ is H, R², R^{2′}, R^{2″}, R³, R⁴, R⁵ and R⁶ are all H and R⁵ is OCH₃,
then the compound of Formula I is an (R)- or (S)-enantiomer of the carbon to which Q is attached.

The present application includes compounds of Formula I:

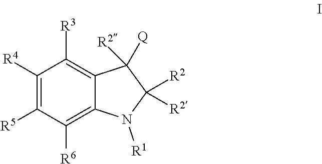

or a pharmaceutically acceptable salt, solvate and/or prodrug thereof,
wherein:
R¹ is selected from H, C(O)R⁷, CO₂R⁷, C(O)N(R⁷)(R^{7′}), S(O)R⁷ and SO₂R⁷;
Q is selected from Q1, Q2, Q3, Q4 and Q5:

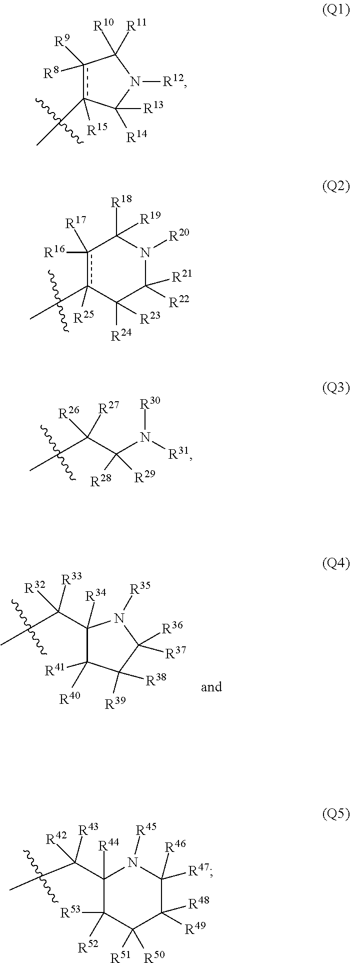

[symbol] is a single bond or a double bond provided that when [symbol] in Q1 is a double bond then R⁹ and R¹⁵ are not present, and when [symbol] in Q2 is a double bond then R¹⁷ and R²⁵ are not present;
R², R^{2′}, R^{2″}, R³ and R⁶ are independently selected from H, halo, C₁₋₆alkyl and C₁₋₆alkoxy;
one or both of R⁴ and R⁵ is independently selected from H, halo, C₁₋₆alkyl and C₁₋₆alkoxy, or
R⁴ and R⁵ are linked together to form O—(CH₂)₁₋₂O, or
one of R⁴ and R⁵ is selected from A, O-A and C₁₋₄alkylene A and the other of R⁴ and R⁵ is H;
A is selected from phenyl, C₃₋₆cycloalkyl, 3- to 6-membered heterocycloalkyl comprising 1 to 4 heteromoeities independently selected from O, S, S(O), SO₂, N and NR⁵⁴ and 5- to 6-membered heteroaryl comprising 1 to 4 heteromoeities independently selected from O, S, S(O), SO₂, N and NR⁵⁴, wherein the phenyl, C₃₋₁₀cycloalkyl, 3- to 6-membered heterocycloalkyl and 5- to 6-membered heteroaryl are optionally substituted with one or more substituents independently selected from halo, C₁₋₄alkyl and OC₁₋₄alkyl;
R⁷ is selected from H and C₁₋₆alkyl, wherein the C₁₋₆alkyl is optionally substituted with one or more substituents independently selected from halo, OR⁵⁵, N(R⁵⁵)(R⁵⁶) and SR⁵⁵ and/or are optionally interrupted by one to three heteromoieties independently selected from O, C(O), CO₂ and NR⁵⁷;
R^{7′} is selected from H and C₁₋₆alkyl;
R⁸, R⁹, R¹⁰, R¹¹, R¹³, R¹⁴, R¹⁵, R¹⁶, R¹⁷, R¹⁸, R¹⁹, R²¹, R²², R²³, R²⁴, R²⁵, R²⁶, R²⁷, R²⁸, R²⁹, R³², R³³, R³⁴, R³⁶, R³⁷, R³⁸, R³⁹, R⁴⁰, R⁴¹, R⁴², R⁴³, R⁴⁴, R⁴⁶, R⁴⁷, R⁴⁸, R⁴⁹, R⁵⁰, R⁵¹, R⁵² and R⁵³ are independently selected from H, halo and C₁₋₆alkyl;
R¹², R²⁰, R³⁵ and R⁴⁵ are independently selected from H, C₁₋₆alkyl and C(O)C₁₋₆alkyl;
R³⁰ and R³¹ are independently selected from H, C₁₋₆alkyl and C(O)C₁₋₆alkyl, or
R³⁰ and R³¹, together with the N atom to which they are bound, form a 3- to 6-membered heterocyclic ring which optionally comprises one or two additional heteromoieties independently selected from O, S, S(O), SO₂, N, and NR⁵⁸;
R⁵⁴, R⁵⁵, R⁵⁶, R⁵⁷ and R⁵⁸ are independently selected from H and C₁₋₆alkyl; and
all available hydrogen atoms are optionally and independently substituted with a fluorine atom or chlorine atom and all available atoms are optionally substituted with alternate isotope thereof,
provided that when Q is Q3, R²⁶, R²⁷, R²⁸ and R²⁹ are all H and R³⁰ and R³¹ are H or CH₃, and:
R¹, R², R^{2′}, R^{2″}, R³, R⁴, R⁵ and R⁶ are all H,
R¹, R², R^{2′}, R^{2″}, R³, R⁵ and R⁶ are all H and R⁴ is OCH₃, or
R¹, R², R^{2′}, R^{2″}, R³, R⁴, R⁵ and R⁶ are all H and R⁵ is OCH₃,
then the compound of Formula I is an (R)- or (S)-enantiomer of the carbon to which Q is attached.

In some embodiments, when all available hydrogen atoms in a group are optionally replaced with a halogen atom, the halogen atom is F, Cl or Br. In some embodiments, when all available hydrogen atoms in a group are optionally replaced with a halogen atom, the halogen atom is F or Br. In some embodiments, when all available hydrogen atoms are replaced with a halogen atom, the halogen atom is F or Cl. In some embodiments, when all available hydrogen atoms in a group are optionally replaced with a halogen atom, the halogen atom is F.

In some embodiments, the compound of Formula I has the following structure:

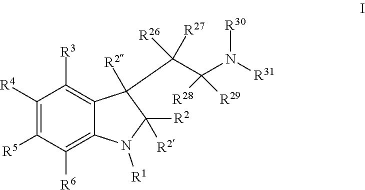

or a pharmaceutically acceptable salt, solvate thereof,
wherein:
R¹, R², R^{2′}, R^{2″}, R³, R⁴, R⁵, R⁶, R²⁶, R²⁷, R²⁸, R²⁹, R³⁰ and R³¹, are as defined for Formula I, and all available hydrogen atoms are optionally and independently substituted with a fluorine atom or chlorine atom and all available atoms are optionally substituted with alternate isotope thereof.

In some embodiments, R¹, R², R^{2′}, R³, R⁵ and R⁶ are all H and the compound Formula I has the following structure:

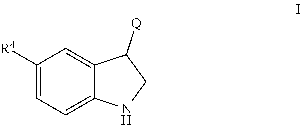

or a pharmaceutically acceptable salt and/or solvate thereof,
wherein:
R⁴ and Q are as defined for Formula I, and
all available hydrogen atoms are optionally and independently substituted with a fluorine atom or chlorine atom and all available atoms are optionally substituted with alternate isotope thereof.

In some embodiments, R¹, R², R^{2′}, R^{2″}, R³, R⁴ and R⁶ are all H and the compound Formula I has the following structure:

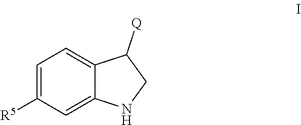

or a pharmaceutically acceptable salt and/or solvate thereof,
wherein:
R⁵ and Q are as defined for Formula I, and
wherein all available hydrogen atoms are optionally and independently substituted with a fluorine atom or chlorine atom and all available atoms are optionally substituted with alternate isotope thereof.

In some embodiments, R¹, R², R^{2′}, R^{2″}, R³ and R⁶ are all H and the compound Formula I has the following structure:

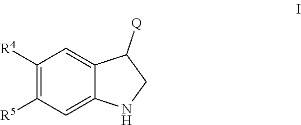

or a pharmaceutically acceptable salt and/or solvate thereof,
wherein:
R⁴, R⁵ and Q are as defined for Formula I, and
all available hydrogen atoms are optionally and independently substituted with a fluorine atom or chlorine atom and all available atoms are optionally substituted with alternate isotope thereof.

In some embodiments, R¹, R², R^{2′} and R^{2″} are all H and R³, R⁴, R⁵ and R⁶ are all D and the compound of Formula I has the following structure:

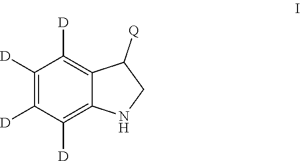

or a pharmaceutically acceptable salt and/or solvate thereof,
wherein:
Q is as defined for Formula I, and
all available hydrogen atoms are optionally and independently substituted with a fluorine atom or chlorine atom and all available atoms are optionally substituted with alternate isotope thereof.

In some embodiments, R²⁶, R²⁷, R²⁸, and R²⁹ are independently selected from H, D, F, C₁, C₁₋₆alkyl, wherein all available hydrogen atoms are optionally and independently substituted with a fluorine atom or deuterium atom. In some embodiments, R²⁶, R²⁷, R²⁸, and R²⁹ are independently selected from H, D, F, Cl, C₁₋₆alkyl, C₁₋₆fluoroalkyl and C₁₋₆deuteroalkyl. In some embodiments, R²⁶, R²⁷, R²⁸, and R²⁹ are independently selected from H, F, D, CH₃, CD₂H, CDH₂, CD₃, CF₃, CHF₂, CH₂CH₃, CH(CH₃)₂, CH₂CH₂D, CH₂CD₂H and CD₂CD₃. In some embodiments, R²⁶, R²⁷, R²⁸, and R²⁹ are independently selected from H, F, D, CH₃, CD₂H, CDH₂, CD₃, CF₃, CHF₂, CH₂CH₃, CH₂CH₂D, CH₂CD₂H and CD₂CD₃. In some embodiments, R²⁶, R²⁷, R²⁸, and R²⁹ are independently selected from H and D.

In some embodiments Q is selected from one of the following groups:

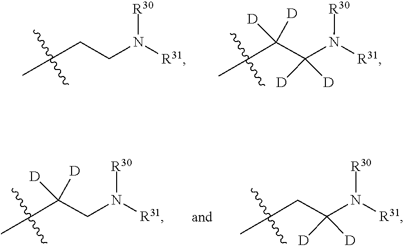

wherein R³⁰ and R³¹ are as defined in Formula I In some embodiments Q is selected from one of the following groups:

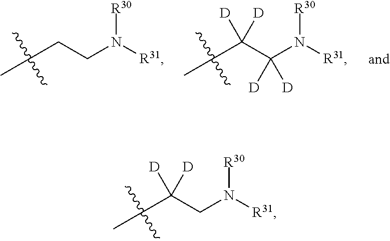

wherein R³⁰ and R³¹ are as defined in Formula I.

In some embodiments, R³⁰ and R³¹ are independently selected from H, C₁₋₆alkyl and C(O)C₁₋₆alkyl, wherein all available hydrogen atoms are optionally and independently substituted with a fluorine atom or deuterium atom. In some embodiments, R³⁰ and R³¹ are independently selected from H, D, C₁₋₆alkyl, C₁₋₆fluoroalkyl, C₁₋₆deuteroalkyl, C(O)C₁₋₆fluoroalkyl and C(O)C₁₋₆deuteroalkyl. In some embodiments, R³⁰ and R³¹ are independently selected from H, D, C₁₋₄alkyl, C₁₋₄fluoroalkyl, C₁₋₄deuteroalkyl, C(O)C₁₋₄alkyl, C(O)C₁₋₄fluoroalkyl and C(O)C₁₋₄deuteroalkyl. In some embodiments, R³⁰ and R³¹ are independently selected from H, D, CH₃, CD₂H, CDH₂, CD₃, CF₃, CHF₂, CH₂CH₃, CH(CH₃)₂, CH₂CH₂D, CH₂CD₂H, CD₂CD₃, CH₃C(O), CD₂HC(O), CDH₂C(O), CD₃C(O), CF₃C(O), CHF₂C(O), CH₃CH₂C(O), CH(CH₃)₂C(O), CH₂DCH₂C(O), CD₂HCH₂C(O) and CD₃CH₂C(O).

In some embodiments, R³⁰ and R³¹ are independently selected from H, D, CH₃, CD₂H, CDH₂, CD₃, CF₃, CHF₂, CH₂CH₃, CH(CH₃)₂, CH₂CH₂D, CH₂CD₂H and CD₂CD₃. In some embodiments, R³⁰ and R³¹ are independently selected from H, D, CH₃, CD₂H, CDH₂, CD₃, CF₃, CHF₂, CH₂CH₃ and CH(CH₃)₂. In some embodiments, R³⁰ and R³¹ are independently selected from H, D, CH₃, CD₃, CF₂H, CF₃ and CH(CH₃)₂. In some embodiments, R³⁰ and R³¹ are independently selected from H, D, CH₃, CF₃ and CH(CH₃). In some embodiments, R³⁰ and R³¹ are independently selected from H, D, CH₃ and CD₃. In some embodiments, R³⁰ and R³¹ are independently selected from H, CH₃ and CD₃. In some embodiments, R³⁰ and R³¹ are independently selected from CH₃ and CD₃. In some embodiments, R³⁰ and R³¹ are independently selected from H and CH(CH₃)₂. In some embodiments, R³⁰ and R³¹ are both CH(CH₃)₂.

In some embodiments, R³⁰ and R³¹ are independently selected from H, D, H, D, CH₃, CD₂H, CDH₂, CD₃, CF₃, CHF₂, CH₃C(O), CD₂HC(O), CDH₂C(O), CD₃C(O), CF₃C(O), CHF₂C(O), CH₃CH₂C(O), CH(CH₃)₂C(O), CH₂DCH₂C(O), CD₂HCH₂C(O) and CD₃CH₂C(O). In some embodiments, R³⁰ and R³¹ are independently selected from H, D, CH₃C(O), CD₂HC(O), CDH₂C(O), CD₃C(O), CF₃C(O), CHF₂C(O), CH₃CH₂C(O), CH(CH₃)₂C(O), CH₂DCH₂C(O), CD₂HCH₂C(O), and CD₃CD₂C(O). In some embodiments, R³⁰ and R³¹ are independently selected from H, CH₃C(O), CD₃C(O), CF₂HC(O) and CF₃C(O).

In some embodiments Q is selected from one of the following groups:

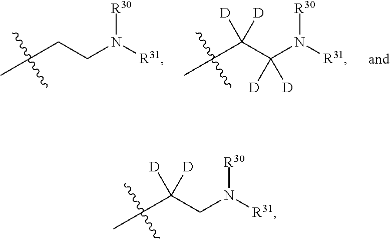

wherein R³⁰ and R³¹ are independently selected from H, D, C₁₋₆alkyl, C₁₋₆fluoroalkyl and C₁₋₁₀ deuteroalkyl. In some embodiments, R³⁰ and R³¹ are independently selected from H, C₁₋₄alkyl, C₁₋₄fluoroalkyl and C₁₋₄deuteroalkyl. In some embodiments, R³⁰ and R³¹ are independently selected from H, CH₃, CD₃, CF₂H and CF₃. In some embodiments, R³⁰ and R³¹ are independently selected from CH₃ and CD₃.

In some embodiments, R³⁰ and R³¹, together with the N atom to which they are bound, form a 3- to 7-membered heterocyclic ring which optionally comprises one or two additional heteromoieties independently selected from O, N and NR⁵⁸. In some embodiments, R³⁰ and R³¹, together with the N atom to which they are bound, form a 3- to 6-membered heterocyclic ring which optionally comprises one or two additional heteromoieties independently selected from O, N and NR⁵⁸.

In some embodiments, the 3- to 6-membered heterocyclic ring is selected from aziridinyl, azetidinyl, pyrrolidinyl, imidazolidinyl, imidazolinyl, pyrazolinyl, piperidinyl, piperazinyl and morpholinyl. In some embodiments, the 3- to 6-membered heterocyclic ring is selected from azetidinyl, pyrrolidinyl, pyrazolinyl, piperidinyl and piperazinyl. In some embodiments, the 3- to 6-membered heterocyclic ring is pyrrolidinyl.

In some embodiments, R¹ is selected from C(O)R⁷, CO₂R⁷, C(O)N(R⁷)(R^{7′}), S(O)R⁷, SO₂R⁷, C₁₋₄alkyleneR⁷ and R⁷, wherein all available hydrogen atoms are optionally and independently substituted with a fluorine atom or deuterium atom.

In some embodiments, R¹ is selected from C(O)R⁷, CO₂R⁷, C(O)N(R⁷)(R⁷), S(O)R⁷ and SO₂R⁷. In some embodiments, R¹ is selected from C(O)R⁷, CO₂R⁷, and C(O)N(R⁷)(R⁷). In some embodiments, R¹ is selected from C(O)R⁷ and CO₂R⁷.

In some embodiments, R^{7′} is selected from H and C₁₋₄alkyl, wherein all available hydrogen atoms are optionally and independently substituted with a fluorine atom or deuterium atom. In some embodiments, R^{7′} is selected from H, D, C₁₋₄alkyl, C₁₋₄fluoroalkyl and C₁₋₄deuteroalkyl. In some embodiments, R^{7′} is selected from H, D, CH₃, CD₂H, CDH₂, CD₃, CF₃, CHF₂, CH₂CH₃, CH(CH₃)₂, CH₂CH₂D, CH₂CD₂H and CD₂CD₃. In some embodiments, R^{7′} is selected from H, CH₃, CD₃, CF₂H and CF₃. In some embodiments, R^{7′} is selected from H, CH₃ and CD₃.

In some embodiments, R¹ is selected from R⁷ and C₁₋₄alkyleneR⁷. In some embodiments, R¹ is selected from R⁷ and C₁₋₂alkyleneR⁷. In some embodiments, R¹ is R⁷. In some embodiments, R¹ is C₁₋₄alkyleneR⁷. In some embodiments, R¹ is C₁alkyleneR⁷ (CH₂R⁷).

In some embodiments, R⁷ is selected from H, C₁₋₄alkyl, C₂₋₆alkenyl, C₂₋₆alkynyl, phenyl, C₃₋₆cycloalkyl, 3- to 6-membered heterocycloalkyl comprising 1 to 4 heteromoeities independently selected from O, S, N and NR^{7″} and 5- to 6-membered heteroaryl comprising 1 to 4 heteromoeities independently selected from O, S, N and NR^{7″}, wherein the latter 7 groups are optionally substituted with one or more substituents independently selected from halo, OR⁵⁵, N(R⁵⁵)(R⁵) and SR⁵⁵ and/or the C₁₋₄alkyl is optionally interrupted by one to three heteromoieties independently selected from O, C(O), CO₂ and NR⁵⁷, and wherein all available hydrogen atoms are optionally and independently substituted with a fluorine atom or deuterium atom.

In some embodiments, R⁷ is selected from phenyl, C₃₋₆cycloalkyl, 3- to 6-membered heterocycloalkyl comprising 1 to 3 heteromoeities independently selected from O, S, S(O), SO₂, N and NR^{7″} and 5- to 6-membered heteroaryl comprising 1 to 3 heteromoeities independently selected from O, S, S(O), SO₂, N and NR^{7″}, optionally substituted with one or more substituents independently selected from halo, OR⁵⁵, N(R⁵⁵)(R⁵⁶) and SR⁵⁵ and wherein all available hydrogen atoms are optionally and independently substituted with a fluorine atom or deuterium atom. In some embodiments, R⁷ is selected from phenyl, C₃₋₆cycloalkyl, 3- to 6-membered heterocycloalkyl comprising 1 to 3 heteromoeities independently selected from O, S, S(O), SO₂, N and NR^{7″} and 5- to 6-membered heteroaryl comprising 1 to 3 heteromoeities independently selected from O, S, S(O), SO₂, N and NR^{7″}, wherein all available hydrogen atoms are optionally and independently substituted with a fluorine atom or deuterium atom. In some embodiments, R⁷ is selected from phenyl, C₃₋₆cycloalkyl, 5- to 6-membered heterocycloalkyl comprising 1 to 2 heteromoeities independently selected from O, S, S(O), SO₂, N and NR^{7″} and 5- to 6-membered heteroaryl comprising 1 to 3 heteromoeities independently selected from O, S, N and NR^{7″}, wherein all available hydrogen atoms are optionally and independently substituted with a fluorine atom or deuterium atom.

In some embodiments, R⁷ is phenyl, optionally substituted with one or more substituents independently selected from halo, OR⁵⁵, N(R⁵⁵)(R⁵) and SR⁵⁵ and wherein all available hydrogen atoms are optionally and independently substituted with a fluorine atom or deuterium atom. In some embodiments, R⁷ is phenyl, wherein all available hydrogen atoms are optionally and independently substituted with a fluorine atom or deuterium atom.

In some embodiments, R⁷ is C₃₋₆cycloalkyl optionally substituted with one or more substituents independently selected from halo, OR⁵⁵, N(R⁵⁵)(R⁵) and SR⁵⁵ and wherein all available hydrogen atoms are optionally and independently substituted with a fluorine atom or deuterium atom. In some embodiments, R⁷ is C₃₋₆cycloalkyl wherein all available hydrogen atoms are optionally and independently substituted with a fluorine atom or deuterium atom. In some embodiments, the C₃₋₆cycloalkyl in R⁷ is selected from cyclopropyl, cyclobutyl, cyclopentyl and cyclohexyl. In some embodiments, the C₃₋₆cycloalkyl in R⁷ is selected from cyclopropyl, cyclobutyl and cyclopentyl. In some embodiment, the C₃₋₆cycloalkyl in R⁷ is cyclopropyl.

In some embodiments, R⁷ is 3- to 6-membered heterocycloalkyl comprising 1 to 3 heteromoeities independently selected from O, S, S(O), SO₂, N and NR^{7″} optionally substituted with one or more substituents independently selected from halo, OR⁵⁵, N(R⁵⁵)(R⁵⁶) and SR⁵⁵ and wherein all available hydrogen atoms are optionally and independently substituted with a fluorine atom or deuterium atom. In some embodiments, R⁷ is 3- to 6-membered heterocycloalkyl comprising 1 to 3 heteromoeities independently selected from O, S, S(O), SO₂, N and NR^{7″} wherein all available hydrogen atoms are optionally and independently substituted with a fluorine atom or deuterium atom. In some embodiments, the 3- to 6-membered heterocycloalkyl comprising 1 to 2 heteromoeities independently selected from O, S, S(O), SO₂, N and NR^{7′} in R⁷ is selected from aziridinyl, oxiranyl, azetidinyl, oxetanyl, pyrrolidinyl, imidazolidinyl, imidazolinyl, pyrazolinyl, tetrahydrofuranyl, piperidinyl, piperazinyl, morpholinyl, tetrahydropyranyl, thianyl, thianyl oxide and thianyl dioxide. In some embodiments, the 3- to 6-membered heterocycloalkyl comprising 1 to 2 heteromoeities independently selected from O, S, S(O), SO₂, N and NR^{7″} in R⁷ is selected from azetidinyl, oxetanyl, pyrrolidinyl, imidazolidinyl, imidazolinyl, pyrazolinyl, tetrahydrofuranyl, piperidinyl, piperazinyl, morpholinyl, tetrahydropyranyl, thianyl, and thianyl dioxide. In some embodiments, the 3- to 6-membered heterocycloalkyl comprising 1 to 2 heteromoeities independently selected from O, S, S(O), SO₂, N and NR^{7″} in R⁷ is selected from tetrahydropyranyl, thianyl, and thianyl dioxide.

In some embodiments, R⁷ is 5- to 6-membered heteroaryl comprising 1 to 3 heteromoeities independently selected from O, S, N and NR^{7′}, optionally substituted with one or more substituents independently selected from halo, OR⁵⁵, N(R⁵⁵)(R⁵⁶) and SR⁵⁵ and wherein all available hydrogen atoms are optionally and independently substituted with a fluorine atom or deuterium atom. In some embodiments, R⁷ is 5- to 6-membered heteroaryl comprising 1 to 3 heteromoeities independently selected from O, S, N and NR^{7″}, wherein all available hydrogen atoms are optionally and independently substituted with a fluorine atom or deuterium atom. In some embodiments, the 5- to 6-membered heteroaryl comprising 1 to 3 heteromoeities independently selected from O, S, N and NR^{7″} in R⁷ is selected from pyrrolyl, furanyl, imidazolyl, oxazolyl, isoxazolyl, isothiazolyl, thiazoyl, pyrazolyl, thiophenyl, pyrazolyl, triazolyl, tetrazolyl, pyridinyl, pyrimidinyl, pyrazinyl and triazinyl. In some embodiments, the 5- to 6-membered heteroaryl comprising 1 to 3 heteromoeities independently selected from O, S, N and NR^{7′} in R⁷ is selected from pyrrolyl, imidazolyl, oxazolyl, isoxazolyl, isothiazolyl, thiazoyl, pyrazolyl, thiophenyl, pyrazolyl and pyridinyl

In some embodiments, R⁷ is selected from C₂₋₆alkenyl and C₂₋₆alkynyl, optionally substituted with one or more substituents independently selected from halo, OR⁵⁵, N(R⁵⁵)(R⁵⁶) and SR⁵⁵ and wherein all available hydrogen atoms are optionally and independently substituted with a fluorine atom or deuterium atom. In some embodiments, R⁷ is selected from C₂₋₄alkenyl and C₂₋₄alkynyl, optionally substituted with one or more substituents independently selected from halo, OR⁵⁵, N(R⁵⁵)(R⁵⁶) and SR⁵⁵ and wherein all available hydrogen atoms are optionally and independently substituted with a fluorine atom or deuterium atom.

In some embodiments, R⁷ is C₂₋₄alkenyl, optionally substituted with one or more substituents independently selected from halo, OR⁵⁵, N(R⁵⁵)(R⁵⁶) and SR⁵⁵ wherein all available hydrogen atoms are optionally and independently substituted with a fluorine atom or deuterium atom. In some embodiments, R⁷ is C₂₋₄alkenyl wherein all available hydrogen atoms are optionally and independently substituted with a fluorine atom or deuterium atom.

In some embodiments, R⁷ is C₂₋₄alkynyl, optionally substituted with one or more substituents independently selected from halo, OR⁵⁵, N(R⁵⁵)(R⁵⁶) and SR⁵⁵ wherein all available hydrogen atoms are optionally and independently substituted with a fluorine atom or deuterium atom. In some embodiments, R⁷ is C₂₋₄alkynyl wherein all available hydrogen atoms are optionally and independently substituted with a fluorine atom or deuterium atom. In some embodiments, R⁷ is selected from C≡CH, C≡CCH₃, CH₂C≡CH, C≡CCH₂CH₃, CH₂C≡CCH₃. In some embodiments, R⁷ is CH₂C≡CH.

In some embodiments, R⁷ is selected from H and C₁₋₄alkyl, wherein the C₁₋₄alkyl is optionally substituted with one or more substituents independently selected from halo, OR⁵⁵, N(R⁵⁵)(R⁵⁶) and SR⁵⁵ and/or the C₁₋₄alkyl is optionally interrupted by one to three heteromoieties independently selected from O, C(O), CO₂ and NR⁵⁷, and wherein all available hydrogen atoms are optionally and independently substituted with a fluorine atom or deuterium atom. In some embodiments, R⁷ is selected from H and C₁₋₄alkyl, wherein the C₁₋₄alkyl is optionally substituted with one to three substituents independently selected from halo, OR⁵⁵, N(R⁵⁵)(R⁵⁶) and SR⁵⁵ and/or the C₁₋₄alkyl is optionally interrupted by one to three heteromoieties independently selected from O, C(O), CO₂ and NR⁵⁷, and wherein all available hydrogen atoms are optionally and independently substituted with a fluorine atom or deuterium atom. In some embodiments, R⁷ is selected from H and C₁₋₄alkyl, wherein the C₁₋₄alkyl is optionally substituted with one to three substituents independently selected from F, Cl, OR⁵⁵, N(R⁵⁵)(R⁵⁶) and SR⁵⁵ and/or the C₁₋₄alkyl is optionally interrupted by one to three heteromoieties independently selected from O, C(O), CO₂ and NR⁵⁷, and wherein all available hydrogen atoms are optionally and independently substituted with a fluorine atom or deuterium atom.

In some embodiments, R⁷ is selected from H and C₁₋₄alkyl, wherein the C₁₋₄alkyl is optionally substituted with one to three substituents independently selected from F, Cl, OR⁵⁵, N(R⁵⁵)(R⁵⁶) and SR⁵⁵ and wherein all available hydrogen atoms are optionally and independently substituted with a fluorine atom or deuterium atom. In some embodiments, R⁷ is selected from H and C₁₋₄alkyl, wherein the C₁₋₄alkyl is optionally substituted OR⁵⁵ and wherein all available hydrogen atoms are optionally and independently substituted with a fluorine atom or deuterium atom.

In some embodiments, R⁷ is selected from H and C₁₋₄alkyl, wherein the C₁₋₄alkyl is optionally interrupted by one to three heteromoieties independently selected from O, C(O), CO₂ and NR⁵⁷, and wherein all available hydrogen atoms are optionally and independently substituted with a fluorine atom or deuterium atom. In some embodiments, R⁷ is selected from H and C₁₋₄alkyl, wherein the C₁₋₄alkyl is optionally interrupted O, and wherein all available hydrogen atoms are optionally and independently substituted with a fluorine atom or deuterium atom.

In some embodiments, R⁷ is selected from H and C₁₋₄alkyl, wherein all available hydrogen atoms are optionally and independently substituted with a fluorine atom or deuterium atom. In some embodiments, R⁷ is selected from H, D, C₁₋₄alkyl, C₁₋₄fluoroalkyl and C₁₋₄deuteroalkyl. In some embodiments, R⁷ is selected from H, D, C₁₋₄alkyl, C₁₋₄fluoroalkyl, C₁₋₄deuteroalkyl, OC₁₋₄alkyl, OC₁₋₄fluoroalkyl and OC₁₋₄deuteroalkyl. In some embodiments, R⁷ is selected from H, D, CH₃, CD₂H, CDH₂, CD₃, CF₃, CHF₂, CH₂CH₃, CH(CH₃)₂, CH₂CH₂D, CH(CD₃)₂, CH₂CD₂H and CD₂CD₃. In some embodiments, R⁷ is selected from H, CH₃, CD₃, CF₂H, CF₃ and CH(CH₃)₂. In some embodiments, R⁷ is selected from H, CH₃, CD₃, CF₂H and CF₃. In some embodiments, R⁷ is selected from H, CH₃, CD₃ and CH(CH₃)₂. In some embodiments, R⁷ is selected from H, CH₃ and CD₃. In some embodiments, R⁷ is H.

In some embodiments, R¹ is R⁷. Therefore, in some embodiments R¹ is selected from H and C₁₋₄alkyl, wherein all available hydrogen atoms are optionally and independently substituted with a fluorine atom or deuterium atom. In some embodiments, R¹ is selected from H, D, C₁₋₄alkyl, C₁₋₄fluoroalkyl and C₁₋₄deuteroalkyl. In some embodiments, R¹ is selected from H, D, C₁₋₄alkyl, C₁₋₄fluoroalkyl, C₁₋₄deuteroalkyl, OC₁₋₄alkyl, OC₁₋₄fluoroalkyl and OC₁₋₄deuteroalkyl. In some embodiments, R¹ is selected from H, D, CH₃, CD₂H, CDH₂, CD₃, CF₃, CHF₂, CH₂CH₃, CH(CH₃)₂, CH₂CH₂D, CH₂CD₂H and CD₂CD₃. In some embodiments, R⁷ is H, D, CH₃, CD₃, CF₃, CH₂CH₃, CH(CD₃)₂, and CH(CH₃)₂ and R¹ is selected from H, D, CH₃, CD₃, CF₃, CH₂CH₃ and CH(CH₃)₂. In some embodiments, R¹ is selected from H, CH₃, CD₃, CF₂H, CF₃ and CH(CH₃)₂. In some embodiments, R¹ is selected from H, CH₃, CD₃, CF₂H and CF₃. In some embodiments, R¹ is selected from H, CH₃, CD₃ and CH(CH₃)₂. In some embodiments, R¹ is R⁷ and R⁷ is selected from H, CH₃, CD₃ and CH(CH₃)₂. In some embodiments, R¹ is selected from H, CH₃ and CD₃. In some embodiments, R¹ is selected from CH₃ and CD₃. In some embodiments, when R¹ is R⁷ and R⁷ is H, and R¹ is H. Therefore, in some embodiments, R¹ is H.

In some embodiments, R^{7″} is selected from H and C₁₋₄alkyl, wherein all available hydrogen atoms are optionally and independently substituted with a fluorine atom or deuterium atom. In some embodiments, R^{7″} is selected from H, D, C₁₋₄alkyl, C₁₋₄fluoroalkyl and C₁₋₄deuteroalkyl. In some embodiments, R^{7″} is selected from H, D, CH₃, CD₂H, CDH₂, CD₃, CF₃, CHF₂, CH₂CH₃, CH(CH₃)₂, CH₂CH₂D, CH₂CD₂H and CD₂CD₃. In some embodiments, R^{7″} is selected from H, CH₃, CD₃, CF₂H and CF₃. In some embodiments, R^{7″} is selected from H, CH₃ and CD₃.

In some embodiments, R², R^{2′} and R^{2″} are independently selected from H, halo, C₁₋₄alkyl C₁₋₄alkoxy, C₁₋₃alkyleneN(R^{2a})(R^{2b}) and SC₁₋₄alkyl, wherein all available hydrogen atoms are optionally and independently substituted with a fluorine atom or deuterium atom.

In some embodiments, R², R^{2′} and R^{2″} are independently selected from H, D, F, Cl, C₁₋₄alkyl, C₁₋₄fluoroalkyl, C₁₋₄deuteroalkyl, C₁₋₄alkoxy, C₁₋₄fluoroalkoxy, C₁₋₄deuteroalkoxy, C₁₋₃ alkyleneN(R^{2a})(R^{2b}), C₁₋₃fluoroalkyleneN(R^{2a})(R^{2b}), C₁₋₃deuteroalkyleneN(R^{2a})(R^{2b}), SC₁₋₄alkyl, SC₁₋₄fluoroalkyl and SC₁₋₄deuteroalkyl. In some embodiments, R², R^{2′} and R^{2″} are independently selected from H, D, F, Cl, C₁₋₄alkyl, C₁₋₄fluoroalkyl, C₁₋₄deuteroalkyl, C₁₋₄alkoxy, C₁₋₄fluoroalkoxy, C₁₋₄deuteroalkoxy, C₁₋₃alkyleneN(R^{2a})(R^{2b}), SC₁₋₄alkyl, SC₁₋₄fluoroalkyl and SC₁₋₄deuteroalkyl.

In some embodiments, R², R^{2′} and R^{2″} are independently selected from H, D, F, C, Br, CH₃, CD₂H, CDH₂, CD₃, CF₃, CHF₂, CH₂CH₃, CH(CH₃)₂, CH₂CH₂D, CH₂CD₂H, CD₂CD₃, CH₃O, CD₂HO, CDH₂O, CD₃O, CF₃O, CHF₂O, CH₂CH₃O, CH(CH₃)₂O, CHD₂CH₂O, CD₂HCH₂O, and CD₃CD₂O, C₁₋₂alkyleneN(R^{2a})(R^{2b}), C₁₋₂fluoroalkyleneN(R^{2a})(R^{2b}), C₁₋₂ deuteroalkyleneN(R^{2a})(R^{2b}), CH₃S, CD₂HS, CDH₂S, CD₃S, CF₃S, CHF₂S, CH₂CH₃S, CH(CH₃)₂S, CHD₂CH₂S, CD₂HCH₂S and CD₃CD₂S. In some embodiments, R², R^{2′} and R^{2″} are independently selected from H, D, F, Cl, Br, CH₃, CD₂H, CDH₂, CD₃, CF₃, CHF₂, CH₂CH₃, CH(CH₃)₂, CH₂CH₂D, CH₂CD₂H, CD₂CD₃, CH₃O, CD₂HO, CDH₂O, CD₃O, CF₃O, CHF₂O, CH₂CH₃O, CH(CH₃)₂O, CHD₂CH₂O, CD₂HCH₂O, and CD₃CD₂O, C₁₋₂alkyleneN(R^{2a})(R^{2b}), CH₃S, CD₂HS, CDH₂S, CD₃S, CF₃S, CHF₂S, CH₂CH₃S, CH(CH₃)₂S, CHD₂CH₂S, CD₂HCH₂S and CD₃CD₂S.

In some embodiments, R², R^{2′} and R^{2″} are independently selected from H, D, F, CH₃, CD₂H, CDH₂, CD₃, CF₃, CHF₂, CH₂CH₃, CH(CH₃)₂, CH₂CH₂D, CH₂CD₂H, CD₂CD₃, CH₃O, CD₂HO, CDH₂O, CD₃O, CF₃O, CHF₂O, CH₂CH₃O, CH(CH₃)₂O, CHD₂CH₂O, CD₂HCH₂O, CD₃CD₂O, CH₃S, CD₂HS, CDH₂S, CD₃S, CF₃S, CHF₂S, CH₂CH₃S, CH(CH₃)₂S, CHD₂CH₂S, CD₂HCH₂S and CD₃CD₂S. In some embodiments, R², R^{2′} and R^{2″} are independently selected from H, D, CH₃S, CD₂HS, CDH₂S, CD₃S, CF₃S, CHF₂S, CH₂CH₃S, CH(CH₃)₂S, CHD₂CH₂S, CD₂HCH₂S and CD₃CD₂S.

In some embodiments, R², R^{2′} and R^{2″} are independently selected from H, D, F, C₁₋₂alkyleneN(R^{2a})(R^{2b}), C₁₋₂fluoroalkyleneN(R^{2a})(R^{2b}) and C₁₋₂deuteroalkyleneN(R^{2a})(R^{2b}).

In some embodiments, R², R^{2′} and R^{2″} are independently selected from H, D, F, Cl, C₁₋₄alkyl, C₁₋₄fluoroalkyl, C₁₋₄deuteroalkyl, C₁₋₄alkoxy, C₁₋₄fluoroalkoxy and C₁₋₄deuteroalkoxy. In some embodiments, R², R^{2′} and R^{2″} are independently selected from H, D, F, Cl, C₁₋₄alkyl, C₁₋₄fluoroalkyl, C₁₋₄deuteroalkyl, C₁₋₄alkoxy, C₁₋₄fluoroalkoxy and C₁₋₄deuteroalkoxy. In some embodiments, R², R^{2′} and R^{2″} are independently selected from H, D, F, CH₃, CD₂H, CDH₂, CD₃, CF₃, CHF₂, CH₂CH₃, CH(CH₃)₂, CH₂CH₂D, CH₂CD₂H, CD₂CD₃, CH₃O, CD₂HO, CDH₂O, CD₃O, CF₃O, CHF₂O, CH₂CH₃O, CH(CH₃)₂O, CHD₂CH₂O, CD₂HCH₂O, and CD₃CD₂O.

In some embodiments, R², R^{2′} and R^{2″} are independently selected from H, F, D, CH₃O, CD₂HO, CDH₂O, CD₃O, CF₃O, CHF₂O, CH₂CH₃O, CH(CH₃)₂O, CH₂DCH₂O, CD₂HCH₂O, and CD₃CD₂O. In some embodiments, R², R^{2′} and R^{2″} are independently selected from H, D, F, CH₃O, CD₃O, CF₂HO and CF₃O.

In some embodiments, R², R^{2′} and R^{2″} are independently selected from H, D, F, CH₃, CD₂H, CDH₂, CD₃, CF₃, CHF₂, CH₂CH₃, CH(CH₃)₂, CH₂CH₂D, CH₂CD₂H and CD₂CD₃. In some embodiments, R², R^{2′} and R^{2″} are independently selected from H, D, F, CH₃, CD₂H, CDH₂, CD₃, CF₃, CHF₂, CH₂CH₃ and CH(CH₃)₂. In some embodiments, R², R^{2′} and R^{2″} are independently selected from H, F, D, CH₃, CD₃, CF₂H and CF₃. In some embodiments, R², R^{2′} and R^{2″} are independently selected from H, F, D, CH₃ and CD₃.

In some embodiments, R^{2″} is selected from H, F, D, CH₃, CD₃, CF₂H and CF₃. In some embodiments, R^{2″} is selected from H, D or F. In some embodiments, R^{2″} is selected from H or F. In some embodiments, R^{2″} is H.

In some embodiments, R² and R^{2′} are independently selected from H, F, D, CH₃, CD₃, CF₂H and CF₃. In some embodiments, R² and R^{2′} are independently selected from H, D or F. In some embodiments, R² and R^{2′} are both H or R² and R^{2′} are both D. In some embodiments, R² and R^{2′} are both H.

In some embodiments, R², R^{2′} and R^{2″} are independently H, D or F. In some embodiments, R², R^{2′} and R^{2″} are all H or are all D. In some embodiments, R², R^{2′} and R^{2″} are all H.

In some embodiments, R^{2a} and R^{2b} are independently selected from H and C₁₋₄alkyl, wherein all available hydrogen atoms are optionally and independently substituted with a fluorine atom or deuterium atom. In some embodiments, R^{2a} and R^{2b} are independently selected from H, D, C₁₋₄alkyl, C₁₋₄fluoroalkyl and C₁₋₄deuteroalkyl. In some embodiments, R^{2a} and R^{2b} are independently selected from H, D, CH₃, CD₂H, CDH₂, CD₃, CF₃, CHF₂, CH₂CH₃, CH(CH₃)₂, CH₂CH₂D, CH₂CD₂H and CD₂CD₃. In some embodiments, R^{2a} and R^{2b} are independently selected from H, CH₃, CD₃, CF₂H and CF₃. In some embodiments, R^{2a} and R^{2b} are independently selected from H, CH₃ and CD₃.

In some embodiments, R³, R⁴, R⁵ and R⁶ are independently selected from H, halo, N(R^{5′})(R^{6′}), C₁₋₄alkyl, C₁₋₄alkoxy, C₁₋₄alkyleneN(R^{5′})(R^{6′}) and SC₁₋₄alkyl, the latter four groups being optionally substituted with one or two substituents selected from OH and C₁₋₄alkoxy wherein all available hydrogen atoms are optionally and independently substituted with a fluorine atom or deuterium atom. In some embodiments, R³, R⁴, R⁵ and R⁶ are independently selected from H, D, F, C, Br, N(R^{5′})(R^{6′}), C₁₋₄alkyl, C₁₋₄fluoroalkyl, C₁₋₄deuteroalkyl, C₁₋₄alkoxy, C₁₋₄fluoroalkoxy, C₁₋₄deuteroalkoxy, C₁₋₄alkyleneN(R^{5′})(R^{6′}), C₁₋₄deuteroalkyleneN(R^{5′})(R^{6′}), C₁₋₄fluorolkyleneN(R^{5′})(R^{6′}), SC₁₋₄alkyl, SC₁₋₄fluoroalkyl and SC₁₋₄deuteroalkyl, the latter twelve groups being optionally substituted with one or two substituents selected from OH, C₁₋₄alkoxy, C₁₋₄fluoroalkoxy and C₁₋₄deuteroalkoxy. In some embodiments, R³, R⁴, R⁵ and R⁶ are independently selected from H, D, F, C, Br, N(R^{5′})(R^{6′}), C₁₋₄alkyl, C₁₋₄fluoroalkyl, C₁₋₄deuteroalkyl, C₁₋₄alkoxy, C₁₋₄fluoroalkoxy, C₁₋₄deuteroalkoxy, C₁₋₄alkyleneN(R^{5′})(R^{6′}), SC₁₋₄ alkyl, SC₁₋₄fluoroalkyl and SC₁₋₄deuteroalkyl, the latter ten groups being optionally substituted with one or two substituents selected from OH, C₁₋₄alkoxy, C₁₋₄fluoroalkoxy and C₁₋₄deuteroalkoxy.

In some embodiments, R³, R⁴, R⁵ and R⁶ are independently selected from H, D, F, C, Br, N(R^{5′})(R^{6′}), C₁₋₄alkyl, C₁₋₄fluoroalkyl, C₁₋₄deuteroalkyl, C₁₋₄alkyleneN(R^{5′})(R^{6′}), SC₁₋₄alkyl, SC₁₋₄fluoroalkyl and SC₁₋₄deuteroalkyl, the latter seven groups being optionally substituted with one or two substituents selected from OH, C₁₋₄alkoxy, C₁₋₄fluoroalkoxy and C₁₋₄deuteroalkoxy.

In some embodiments, R³, R⁴, R⁵ and R⁶ are independently selected from H, D, N(R^{5′})(R^{6′}), C₁₋₄alkyl, C₁₋₄fluoroalkyl, C₁₋₄deuteroalkyl, C₁₋₄alkyleneN(R^{5′})(R^{6′}), SC₁₋₄alkyl, SC₁₋₄fluoroalkyl and SC₁₋₄deuteroalkyl.

In some embodiments, R³, R⁴, R⁵ and R⁶ are independently selected from H, D, F, C, Br, N(R^{5′})(R^{6′}), C₁₋₄alkyl, C₁₋₄fluoroalkyl, C₁₋₄deuteroalkyl and C₁₋₄alkyleneN(R^{5′})(R^{6′}), the latter four groups being optionally substituted with one or two substituents selected from OH, C₁₋₄alkoxy, C₁₋₄fluoroalkoxy and C₁₋₄deuteroalkoxy. In some embodiments, R³, R⁴, R⁵ and R⁶ are independently selected from H, D, F, C, Br, N(R^{5′})(R^{6′}), C₁₋₄alkyl, C₁₋₄fluoroalkyl, C₁₋₄deuteroalkyl and C₁₋₂alkyleneN(R^{5′})(R^{6′}). In some embodiments, one to three of R³, R⁴, R⁵ and R⁶ are independently selected from H, D, F, N(R^{5′})(R^{6′}), CH₃, CD₂H, CDH₂, CD₃, CF₃, CHF₂, CH₂CH₃, CH(CH₃)₂, CH₂CH₂D, CH₂CD₂H, CD₂CD₃, and C₁₋₂alkyleneN(R^{5′})(R^{6′}) and the remaining of R³, R⁴, R⁵ and R⁶ are selected from H and D. In some embodiments, one to three of R³, R⁴, R⁵ and R⁶ are independently selected from H, D, F, N(R^{5′})(R^{6′}), CH₃, CD₂H, CDH₂, CD₃, CF₃, CHF₂, CH₂CH₃, CH(CH₃)₂, CH₂CH₂D, CH₂CD₂H, CD₂CD₃, and CH₂alkyleneN(R^{5′})(R^{6′}) and the remaining of R³, R⁴, R⁵ and R⁶ are selected from H and D. In some embodiments, R³, R⁴, R⁵ and R⁶ are independently selected from H, D, N(R^{5′})(R^{6′}), C₁₋₄alkyl, C₁₋₄fluoroalkyl, C₁₋₄deuteroalkyl and C₁₋₂alkyleneN(R^{5′})(R^{6′}). In some embodiments, one to three of R³, R⁴, R⁵ and R⁶ are independently selected from H, D, N(R^{5′})(R^{6′}), CH₃, CD₂H, CDH₂, CD₃, CF₃, CHF₂, CH₂CH₃, CH(CH₃)₂, CH₂CH₂D, CH₂CD₂H, CD₂CD₃, and C₁₋₂ alkyleneN(R^{5′})(R^{6′}) and the remaining of R³, R⁴, R⁵ and R⁶ are selected from H and D.

In some embodiments, R³, R⁴, R⁵ and R⁶ are independently selected from H, D, F, C, Br, C₁₋₄alkyl, C₁₋₄fluoroalkyl, C₁₋₄deuteroalkyl, SC₁₋₄alkyl, SC₁₋₄fluoroalkyl and SC₁₋₄deuteroalkyl, the latter six groups being optionally substituted with one or two substituents selected from OH, C₁₋₄alkoxy, C₁₋₄fluoroalkoxy and C₁₋₄deuteroalkoxy. In some embodiments, one to three of R³, R⁴, R⁵ and R⁶ are independently selected from H, D, F, Cl, C₁₋₃alkyl, C. 3fluoroalkyl, C₁₋₃deuteroalkyl, SC₁₋₃alkyl, SC₁₋₃fluoroalkyl and SC₁₋₃deuteroalkyl, and the remaining of R³, R⁴, R⁵ and R⁶ are selected from H and D. In some embodiments, one to three of R³, R⁴, R⁵ and R⁶ are independently selected from H, D, F, CH₃, CD₂H, CDH₂, CD₃, CF₃, CHF₂, CH₂CH₃, CH(CH₃)₂, CH₂CH₂D, CH₂CD₂H, CD₂CD₃, CH₃S, CD₂HS, CDH₂S, CD₃S, CF₃S, CHF₂S, CH₂CH₃S, CH(CH₃)₂S, CHD₂CH₂S, CD₂HCH₂S and CD₃CD₂S and the remaining of R³, R⁴, R⁵ and R⁶ are selected from H and D. In some embodiments, one to three of R³, R⁴, R⁵ and R⁶ are independently selected from H, D, C₁₋₃alkyl, C₁₋₃fluoroalkyl, C₁₋₃ deuteroalkyl, SC₁₋₃alkyl, SC₁₋₃fluoroalkyl and SC₁₋₃deuteroalkyl, and the remaining of R³, R⁴, R⁵ and R⁶ are selected from H and D. In some embodiments, one to three of R³, R⁴, R⁵ and R⁶ are independently selected from H, D, CH₃, CD₂H, CDH₂, CD₃, CF₃, CHF₂, CH₂CH₃, CH(CH₃)₂, CH₂CH₂D, CH₂CD₂H, CD₂CD₃, CH₃S, CD₂HS, CDH₂S, CD₃S, CF₃S, CHF₂S, CH₂CH₃S, CH(CH₃)₂S, CHD₂CH₂S, CD₂HCH₂S and CD₃CD₂S and the remaining of R³, R⁴, R⁵ and R⁶ are selected from H and D.

In some embodiments, R³, R⁴, R⁵ and R⁶ are independently selected from H, halo, C₁₋₄alkyl and C₁₋₄alkoxy, the latter two groups being optionally substituted with one or two substituents selected from OH and C₁₋₄alkoxy wherein all available hydrogen atoms are optionally and independently substituted with a fluorine atom or deuterium atom. In some embodiments, R³, R⁴, R⁵ and R⁶ are independently selected from H, D, F, Cl, Br, C₁₋₄alkyl, C₁₋₄fluoroalkyl, C₁₋₄deuteroalkyl, C₁₋₄alkoxy, C₁₋₄fluoroalkoxy and C₁₋₄deuteroalkoxy, the latter six groups being optionally substituted with one or two substituents selected from OH, C₁₋₄alkoxy, C₁₋₄fluoroalkoxy and C₁₋₄deuteroalkoxy.

In some embodiments, R³, R⁴, R⁵ and R⁶ are independently selected from H, D, F, Cl, Br, C₁₋₄alkoxy, C₁₋₄fluoroalkoxy and C₁₋₄deuteroalkoxy, the latter three groups being substituted with one or two substituents selected from OH, C₁₋₄alkoxy, C₁₋₄fluoroalkoxy and C₁₋₄deuteroalkoxy. In some embodiments, one of R³, R⁴, R⁵ and R⁶ is selected from C₁₋₄alkoxy, C₁₋₄fluoroalkoxy and C₁₋₄deuteroalkoxy substituted with one or two substituents selected from OH, C₁₋₄alkoxy, C₁₋₄fluoroalkoxy and C₁₋₄deuteroalkoxy, and the remaining of R³, R⁴, R⁵ and R⁶ are independently selected from H, D, F, Cl, Br, C₁₋₄alkoxy, C₁₋₄fluoroalkoxy and C₁₋₄deuteroalkoxy. In some embodiments, one of R³, R⁴, R⁵ and R⁶ is selected from C₁₋₃alkoxy, C₁₋₃fluoroalkoxy and C₁₋₃deuteroalkoxy substituted with one or two substituents selected from OH, C₁₋₃alkoxy, C₁₋₃fluoroalkoxy and C₁₋₃deuteroalkoxy, and the remaining of R³, R⁴, R⁵ and R⁶ are independently selected from H, D, CH₃O, CD₃O, and CF₃O. In some embodiments, one of R³ and R⁴ is selected from C₁₋₃alkoxy, C₁₋₃fluoroalkoxy and C₁₋₃deuteroalkoxy substituted with one or two substituents selected from OH, C₁₋₃alkoxy, C₁₋₃fluoroalkoxy and C₁₋₃deuteroalkoxy, and the remaining of R³, R⁴, R⁵ and R⁶ are independently selected from H, D, CH₃O, CD₃O and CF₃O. In some embodiments, one of R³ and R⁴ is selected from C₁₋₃alkoxy substituted with C₁₋₃alkoxy and the remaining of R³, R⁴, R⁵ and R⁶ are independently selected from H, D, CH₃O, CD₃O and CF₃O. In some embodiments, one of R³ and R⁴ is C₁₋₃alkoxy substituted with one substituent selected from CH₃O, CD₃O and CF₃O, and the remaining of R³, R⁴, R⁵ and R⁶ are independently selected from H, D, CH₃O, CD₃O and CF₃O. In some embodiments, R⁴ is C₁₋₃alkoxy substituted with one substituent selected from CH₃O, CD₃O and CF₃O, and the remaining of R³, R⁵ and R⁶ are independently selected from H, D, CH₃O, CD₃O and CF₃O.

In some embodiments, R³, R⁴, R⁵ and R⁶ are independently selected from H, D, F, Cl, Br, C₁₋₄alkyl, C₁₋₄fluoroalkyl, C₁₋₄deuteroalkyl, C₁₋₄alkoxy, C₁₋₄fluoroalkoxy and C₁₋₄deuteroalkoxy. In some embodiments, one to three of R³, R⁴, R⁵ and R⁶ are independently selected from H, D, F, Cl, Br, C₁₋₃alkyl, C₁₋₃fluoroalkyl, C₁₋₃deuteroalkyl, C₁₋₃alkoxy, C₁₋₄fluoroalkoxy and C₁₋₄deuteroalkoxy and the remaining of R³, R⁴, R⁵ and R⁶ are selected from H and D.

In some embodiments, one to three of R³, R⁴, R⁵ and R⁶ are independently selected from H, D, F, Cl, Br, CH₃, CD₂H, CDH₂, CD₃, CF₃, CHF₂, CH₂CH₃, CH(CH₃)₂, CH₂CH₂D, CH₂CD₂H, CD₂CD₃, CH₃O, CD₂HO, CDH₂O, CD₃O, CF₃O, CHF₂O, CH₃CH₂O, CH(CH₃)₂O, CH₂DCH₂O, CD₂HCH₂O and CD₃CD₂O and the remaining of R³, R⁴, R⁵ and R⁶ are selected from H and D.

In some embodiments, one to three of R³, R⁴, R⁵ and R⁶ are independently selected from H, D, F, CH₃O, CD₂HO, CDH₂O, CD₃O, CF₃O, CHF₂O, CH₂CH₃O, CH(CH₃)₂O, CH₂DCH₂O, CD₂HCH₂O and CD₃CD₂O and the remaining of R³, R⁴, R⁵ and R⁶ are selected from H and D.

In some embodiments, one to three of R³, R⁴, R⁵ and R⁶ are F and the remaining of R³, R⁴, R⁵ and R⁶ are independently selected from H and D. In some embodiments, one or two of R³, R⁴, R⁵ and R⁶ are F and the remaining of R³, R⁴, R⁵ and R⁶ are independently selected from H and D. In some embodiments, one of R³ and R⁴ is F and the R⁵ remaining of R³, R⁴, R⁵ and R⁶ are independently selected from H and D.

In some embodiments, one to three of R³, R⁴, R⁵ and R⁶ are independently selected from CH₃O, CD₂HO, CDH₂O, CD₃O, CF₃O, CHF₂O, CH₂CH₃O, CH(CH₃)₂O, CH₂DCH₂O, CD₂HCH₂O and CD₃CD₂O and the remaining of R³, R⁴, R⁵ and R⁶ are independently selected from H and D. In some embodiments, one to three of R³, R⁴, R⁵ and R⁶ are independently selected from CH₃O, CD₂HO, CDH₂O, CD₃O, CF₃O and CHF₂O, and the remaining of R³, R⁴, R⁵ and R⁶ are independently selected from H and D. In some embodiments, one or two of R³, R⁴, R⁵ and R⁶ are independently selected from CH₃O, CD₂HO, CDH₂O, CD₃O, CF₃O and CHF₂O, and the remaining of R³, R⁴, R⁵ and R⁶ are independently selected from H and D.

In some embodiments, one or both of R⁴ and R⁵ are independently selected from H, F, CH₃O, CD₂HO, CDH₂O, CD₃O, CF₃O, CHF₂O, CH₂CH₃O, CH(CH₃)₂O, CH₂DCH₂O, CD₂HCH₂O and CD₃CD₂O and the remaining of R³, R⁴, R⁵ and R⁶ are selected from H and D.

In some embodiments, one or both of R⁴ and R⁵ are independently selected from CH₃O, CD₂HO, CDH₂O, CD₃O, CF₃O, CHF₂O, CH₂CH₃O, CH(CH₃)₂O, CH₂DCH₂O, CD₂HCH₂O and CD₃CD₂O and the remaining of R³, R⁴, R⁵ and R⁶ are selected from H and D. In some embodiments, one or both of R⁴ and R⁵ are independently selected from OCH₃, OCD₂H, OCDH₂, OCD₃, OCF₃ and OCHF₂, and the remaining of R³, R⁴, R⁵ and R⁶ are independently selected from H and D. In some embodiments, both of R⁴ and R⁵ are independently selected from OCH₃, OCD₂H, OCDH₂, OCD₃, OCF₃ and OCHF₂, and R³ and R⁶ are independently selected from H and D.

In some embodiments, R³, R⁴, R⁵ and R⁶ are independently selected from H, D, F, CH₃, CD₂H, CDH₂, CD₃, CF₃, CHF₂, CH₂CH₃, CH(CH₃)₂, CH₂CH₂D, CH₂CD₂H and CD₂CD₃ and the remaining of R³, R⁴, R⁵ and R⁶ are independently selected from H and D. In some embodiments, one or two of R³, R⁴, R⁵ and R⁶ are independently selected from CH₃, CD₂H, CDH₂, CD₃, CF₃, CHF₂, CH₂CH₃, CH(CH₃)₂, CH₂CH₂D, CH₂CD₂H and CD₂CD₃ and the remaining of R³, R⁴, R⁵ and R⁶ are independently selected from H and D. In some embodiments, one or both of R⁴ and R⁵ are independently selected from CH₃, CD₂H, CDH₂, CD₃, CF₃, CHF₂, CH₂CH₃, CH(CH₃)₂, CH₂CH₂D, CH₂CD₂H and CD₂CD₃ and the remaining of R³, R⁴, R⁵ and R⁶ are independently selected from H and D. In some embodiments, R³, R⁴, R⁵ and R⁶ are selected from H and D. In some embodiments, all of R³, R⁴, R⁵ and R⁶ are D or all of R³, R⁴, R⁵ and R⁶ are H.

In some embodiments, R³ and R⁶ are independently selected from H, D, C₁, F, C₁₋₄alkyl, C₁₋₄fluoroalkyl and C₁₋₄deuteroalkyl. In some embodiments, R³ and R⁶ are independently selected from H, D, C, F, C₁₋₂alkyl, C₁₋₂fluoroalkyl and C₁₋₂deuteroalkyl. In some embodiments, R³ and R⁶ are independently selected from H, D, F, C, Br, CH₃, CD₂H, CDH₂, CD₃, CF₃ and CHF₂. In some embodiments, R³ and R⁶ are independently selected from H, D, F, CH₃, CF₃ and CD₃.

In some embodiments, R³ and R⁶ are independently selected from H and D. In some embodiments, at least one of R³ and R⁶ is D. In some embodiments, each of R³ and R⁶ are D. In some embodiments, each of R³ and R⁶ are H.

In some embodiments, one or both of R⁴ and R⁵ is independently selected from H, D, F, Cl, C₁₋₆alkyl, C₁₋₆fluoroalkyl, C₁₋₆deuteroalkyl, C₁₋₆alkoxy, C₁₋₆fluoroalkoxy and C₁₋₆ deuteroalkoxy. In some embodiments, one or both of R⁴ and R⁵ is independently selected from H, D, F, C, CH₃, CF₃, CF₂H, CD₃, CH₃O, CF₃O, CHF₂O, and CD₃O.

In some embodiments both of R⁴ and R⁵ are independently selected from D, F, Cl, C₁₋₆alkyl, C₁₋₆fluoroalkyl, C₁₋₆deuteroalkyl, C₁₋₆alkoxy, C₁₋₆fluoroalkoxy and C₁₋₆deuteroalkoxy. In some embodiments both of R⁴ and R⁵ are independently selected from D, F, C, CH₃, CF₃, CF₂H, CD₃, CH₃O, CF₃O, CHF₂O, and CD₃O.

In some embodiments, R⁴ is H or D and R⁵ is selected from H, D, F, Cl, C₁₋₆alkyl, C₁₋₆ fluoroalkyl, C₁₋₆deuteroalkyl, C₁₋₆alkoxy, C₁₋₆fluoroalkoxy and C₁₋₆deuteroalkoxy. In some embodiments, R⁴ is H or D and R⁵ is selected from D, F, C, CH₃, CF₃, CF₂H, CD₃, CH₃O, CF₃O, CHF₂O, and CD₃O.

In some embodiments, R⁵ is H or D and R⁴ is selected from H, D, F, Cl, C₁₋₆alkyl, C₁₋₆ fluoroalkyl, C₁₋₆deuteroalkyl, C₁₋₆alkoxy, C₁₋₆fluoroalkoxy and C₁₋₆deuteroalkoxy. In some embodiments, R⁵ is H or D and R⁴ is selected from D, F, Cl, CH₃, CF₃, CF₂H, CD₃, CH₃O, CF₃O, CHF₂O, and CD₃O.

In some embodiments, two adjacent of R³, R⁴, R⁵ and R⁶ are linked together to form O—(CH₂)₁₋₂O and the remaining of R³, R⁴, R⁵ and R⁶ are independently selected from H, halo, C₁₋₄alkyl, C₁₋₄alkoxy, N(R^{5′})(R^{6′}), C₁₋₄alkyleneN(R^{5′})(R^{6′}) and SC₁₋₄alkyl, wherein all available hydrogen atoms are optionally and independently substituted with a fluorine atom or deuterium atom. In some embodiments, two adjacent of R³, R⁴, R⁵ and R⁶ are linked together to form O—(CH₂)₁₋₂O and the remaining of R³, R⁴, R⁵ and R⁶ are independently selected from H, F, Cl, Br, C₁₋₄alkyl, C₁₋₄fluoroalkyl, C₁₋₄deuteroalkyl, C₁₋₄alkoxy, C₁₋₄fluoroalkoxy, C₁₋₄deuteroalkoxy, N(R^{5′})(R^{6′}), C₁₋₄alkyleneN(R^{5′})(R^{6′}), C₁₋₄deuteroalkyleneN(R^{5′})(R^{6′}), C₁₋₄fluorolkyleneN(R^{5′})(R^{6′}), SC₁₋₄alkyl, SC₁₋₄fluoroalkyl and SC₁₋₄deuteroalkyl. In some embodiments, two adjacent of R³, R⁴, R⁵ and R⁶ are linked together to form O—(CH₂)₁₋₂O and the remaining of R³, R⁴, R⁵ and R⁶ are independently selected from H, F, Cl, Br, C₁₋₄alkyl, C₁₋₄fluoroalkyl, C₁₋₄deuteroalkyl, C₁₋₄alkoxy, C₁₋₄fluoroalkoxy, C₁₋₄deuteroalkoxy, N(R^{5′})(R^{6′}), C₁₋₄alkyleneN(R^{5′})(R^{6′}), SC₁₋₄alkyl, SC₁₋₄fluoroalkyl and SC₁₋₄deuteroalkyl.

In some embodiments, two adjacent of R³, R⁴, R⁵ and R⁶ are linked together to form O—(CH₂)₁₋₂O and the remaining of R³, R⁴, R⁵ and R⁶ are independently selected from H, D, F, Cl, Br, C₁₋₄alkyl, C₁₋₄fluoroalkyl, C₁₋₄deuteroalkyl, N(R^{5′})(R^{6′}) and C₁₋₄alkyleneN(R^{5′})(R^{6′}). In some embodiments, two adjacent of R³, R⁴, R⁵ and R⁶ are linked together to form O—(CH₂)₁₋₂O and the remaining of R³, R⁴, R⁵ and R⁶ are independently selected from H, D, F, Cl, Br, C₁₋₄alkyl, C₁₋₄fluoroalkyl, C₁₋₄deuteroalkyl, N(R^{5′})(R^{6′}), and C₁₋₂alkyleneN(R^{5′})(R^{6′}). In some embodiments, two adjacent of R³, R⁴, R⁵ and R⁶ are linked together to form O—(CH₂)₁₋₂O and the remaining of R³, R⁴, R⁵ and R⁶ are independently selected from H, D, F, CH₃, CD₂H, CDH₂, CD₃, CF₃, CHF₂, CH₂CH₃, CH(CH₃)₂, CH₂CH₂D, CH₂CD₂H, CD₂CD₃, N(R^{5′})(R^{6′}) and CH₂alkyleneN(R^{5′})(R^{6′}). In some embodiments, R⁴ and R⁵ are linked together to form O—(CH₂)O and R³ and R⁶ are independently selected from H, D, F, CH₃, CD₂H, CDH₂, CD₃, CF₃, CHF₂, CH₂CH₃, CH(CH₃)₂, CH₂CH₂D, CH₂CD₂H, CD₂CD₃, N(R^{5′})(R^{6′}) and CH₂alkyleneN(R^{5′})(R^{6′}).

In some embodiments, two adjacent of R³, R⁴, R⁵ and R⁶ are linked together to form O—(CH₂)₁₋₂O and the remaining of R³, R⁴, R⁵ and R⁶ are independently selected from H, D, F, Cl, Br, C₁₋₄alkyl, C₁₋₄fluoroalkyl, C₁₋₄deuteroalkyl, SC₁₋₄alkyl, SC₁₋₄fluoroalkyl and SC₁₋₄ deuteroalkyl. In some embodiments, two adjacent of R³, R⁴, R⁵ and R⁶ are linked together to form O—(CH₂)₁₋₂O and the remaining of R³, R⁴, R⁵ and R⁶ are independently selected from H, D, F, Cl, C₁₋₃alkyl, C₁₋₃fluoroalkyl, C₁₋₃deuteroalkyl, SC₁₋₃alkyl, SC₁₋₃fluoroalkyl and SC₁₋₃ deuteroalkyl. In some embodiments, two adjacent of R³, R⁴, R⁵ and R⁶ are linked together to form O—(CH₂)₁₋₂O and the remaining of R³, R⁴, R⁵ and R⁶ are independently selected from H, D, F, CH₃, CD₂H, CDH₂, CD₃, CF₃, CHF₂, CH₂CH₃, CH(CH₃)₂, CH₂CH₂D, CH₂CD₂H, CD₂CD₃, CH₃S, CD₂HS, CDH₂S, CD₃S, CF₃S, CHF₂S, CH₂CH₃S, CH(CH₃)₂S, CHD₂CH₂S, CD₂HCH₂S and CD₃CD₂S. In some embodiments, R⁴ and R⁵ are linked together to form O—(CH₂)O and R³ and R⁶ are independently selected from H, D, F, CH₃, CD₂H, CDH₂, CD₃, CF₃, CHF₂, CH₂CH₃, CH(CH₃)₂, CH₂CH₂D, CH₂CD₂H, CD₂CD₃, CH₃S, CD₂HS, CDH₂S, CD₃S, CF₃S, CHF₂S, CH₂CH₃S, CH(CH₃)₂S, CHD₂CH₂S, CD₂HCH₂S and CD₃CD₂S.

In some embodiments, R^{5′} and R^{6′} are independently selected from H and C₁₋₄alkyl, wherein all available hydrogen atoms are optionally and independently substituted with a fluorine atom or deuterium atom. In some embodiments, R^{5′} and R^{6′} are independently selected from H, D, C₁₋₄alkyl, C₁₋₄fluoroalkyl and C₁₋₄deuteroalkyl. In some embodiments, R^{5′} and R^{6′} are independently selected from H, D, CH₃, CD₂H, CDH₂, CD₃, CF₃, CHF₂, CH₂CH₃, CH(CH₃)₂, CH₂CH₂D, CH₂CD₂H and CD₂CD₃. In some embodiments, R^{5′} and R^{6′} are independently selected from H, CH₃, CD₃, CF₂H and CF₃. In some embodiments, R^{5′} and R^{6′} are independently selected from H, CH₃ and CD₃.

In some embodiments, two adjacent of R³, R⁴, R⁵ and R⁶ are linked together to form O—(CH₂)₁₋₂O and the remaining of R³, R⁴, R⁵ and R⁶ are independently selected from H, halo, C₁₋₆alkyl and C₁₋₆alkoxy, wherein all available hydrogen atoms are optionally and independently substituted with a fluorine atom or deuterium atom. In some embodiments, two adjacent of R³, R⁴, R⁵ and R⁶ are linked together to form O—(CH₂)₁₋₂O and the remaining of R³, R⁴, R⁵ and R⁶ independently selected from H, halo, C₁₋₄alkyl and C₁₋₄alkoxy, wherein all available hydrogen atoms are optionally and independently substituted with a fluorine atom or deuterium atom. In some embodiments, two adjacent of R³, R⁴, R⁵ and R⁶ are linked together to form O—(CH₂)₁₋₂O and the remaining of R³, R⁴, R⁵ and R⁶ are independently selected from H, F, C, Br, C₁₋₄alkyl, C₁₋₄fluoroalkyl, C₁₋₄deuteroalkyl, C₁₋₄alkoxy, C₁₋₄fluoroalkoxy and C₁₋₄deuteroalkoxy. In some embodiments, two adjacent of R³, R⁴, R⁵ and R⁶ are linked together to form O—(CH₂)₀ and the remaining of R³, R⁴, R⁵ and R⁶ are independently selected from H, D, F, C, Br, CH₃, CD₂H, CDH₂, CD₃, CF₃, CHF₂, CH₂CH₃, CH(CH₃)₂, CH₂CH₂D, CH₂CD₂H, CD₂CD₃, CH₃O, CD₂HO, CDH₂O, CD₃O, CF₃O, CHF₂O, CH₃CH₂O, CH(CH₃)₂O, CH₂DCH₂O, CD₂HCH₂O and CD₃CD₂O.

In some embodiments, R⁴ and R⁵ are linked together to form O—(CH₂)O and R³ and R⁶ are independently selected from H, F, C, Br, C₁₋₄alkyl, C₁₋₄fluoroalkyl, C₁₋₄deuteroalkyl, C₁₋₄alkoxy, C₁₋₄fluoroalkoxy and C₁₋₄deuteroalkoxy. In some embodiments, R⁴ and R⁵ are linked together to form O—(CH₂)O and R³ and R⁶ are independently selected from H, D, F, C, Br, CH₃, CD₂H, CDH₂, CD₃, CF₃, CHF₂, CH₂CH₃, CH(CH₃)₂, CH₂CH₂D, CH₂CD₂H, CD₂CD₃, CH₃O, CD₂HO, CDH₂O, CD₃O, CF₃O, CHF₂O, CH₃CH₂O, CH(CH₃)₂O, CH₂DCH₂O, CD₂HCH₂O and CD₃CD₂O. In some embodiments, R⁴ and R⁵ are linked together to form O—(CH₂)O and R³ and R⁶ are independently selected from H, D, F, CH₃O, CD₂HO, CDH₂O, CD₃O, CF₃O and CHF₂O. In some embodiments, R⁴ and R⁵ are linked together to form O—(CH₂)O and R³ and R⁶ are both H or R³ and R⁶ are both D. In some embodiments, R⁴ and R⁵ are linked together to form O—(CH₂)O and R³ and R⁶ are both H.

In some embodiments, R⁴ and R⁵ are linked together to form O—CH₂O.

In some embodiments, one of R³, R⁴, R⁵ and R⁶ is selected from A, O-A and C₁₋₂alkyleneA and the remaining of R³, R⁴, R⁵ and R⁶ are independently H or halo, wherein all available hydrogen atoms are optionally and independently substituted with a fluorine atom or deuterium atom. In some embodiments, one of R³, R⁴, R⁵ and R⁶ is selected from A, O-A and C₁₋₂alkyleneA and the remaining of R³, R⁴, R⁵ and R⁶ are independently H, F, Br or Cl, wherein all available hydrogen atoms are optionally and independently substituted with a fluorine atom or deuterium atom. In some embodiments, one of R³, R⁴, R⁵ and R⁶ is selected from A, O-A, CH₂A, CF₂A and CD₂A and the remaining of R³, R⁴, R⁵ and R⁶ are independently H, D or F. In some embodiments, one of R³, R⁴, R⁵ and R⁶ is selected from A, O-A, CH₂A, CF₂A and CD₂A and the remaining of R³, R⁴, R⁵ and R⁶ are independently selected from H and D.

In some embodiments, one of R³, R⁴ and R⁵ is selected from A, O-A and C₁₋₂alkyleneA and the remaining of R³, R⁴, R⁵ and R⁶ are independently H or halo, wherein all available hydrogen atoms are optionally and independently substituted with a fluorine atom or deuterium atom. In some embodiments, one of R³, R⁴ and R⁵ is selected from A, O-A, CH₂A, CF₂A and CD₂A and the remaining of R³, R⁴, R⁵ and R⁶ are independently selected from H, Cl, F, Br, D and F. In some embodiments, one of R³, R⁴ and R⁵ is selected from A, O-A, CH₂A, CF₂A and CD₂A and the remaining of R³, R⁴, R⁵ and R⁶ are independently selected from H, D and F.

In some embodiments, one of R⁴ and R⁵ is selected from A, O-A and C₁₋₂alkylene A and the other of R⁴ and R⁵ is H or D, wherein all available hydrogen atoms are optionally and independently substituted with a fluorine atom or deuterium atom. In some embodiments, one of R⁴ and R⁵ is selected from A, O-A, CH₂A, CF₂A and CD₂A and the other of R⁴ and R⁵ is H or D. In some embodiments, R⁴ is selected from A, O-A and C₁₋₂alkylene A and R⁵ is H or D, wherein all available hydrogen atoms are optionally and independently substituted with a fluorine atom or deuterium atom. In some embodiments, R⁴ is selected from A, O-A, CH₂A, CF₂A and CD₂A and R⁵ is H or D. In some embodiments, R⁵ is selected from A, O-A and C₁₋₂ alkylene A and R⁴ is H or D, wherein all available hydrogen atoms are optionally and independently substituted with a fluorine atom or deuterium atom. In some embodiments, R⁵ is selected from A, O-A, CH₂A, CF₂A and CD₂A and R⁴ is H or D.

In some embodiments, A is selected from phenyl, C₃₋₆cycloalkyl, 3- to 6-membered heterocycloalkyl comprising 1 to 3 heteromoeities independently selected from O, S, S(O), SO₂, N and NR⁵⁴ and 5- to 6-membered heteroaryl comprising 1 to 3 heteromoeities independently selected from O, S, S(O), SO₂, N and NR⁵⁴, wherein the phenyl, C₃₋₁₀cycloalkyl, 3- to 6-membered heterocycloalkyl and 5- to 6-membered heteroaryl are optionally substituted with one or two substituents independently selected from F, Cl, Br, C₁₋₄alkyl, C₁₋₄deuteroalkyl, C₁₋₄fluoroalkyl, OC₁₋₄alkyl, OC₁₋₄deuteroalkyl, OC₁₋₄fluoroalkyl, N(R^{54a})(R^{54b}), C₁₋₄ alkyleneN(R^{54a})(R^{54b}), C₁₋₄deuteroalkyleneN(R^{54a})(R^{54b}), C₁₋₄fluoroalkyleneN(R^{54a})(R^{54b}), SC₁₋₄ alkyl, SC₁₋₄deuteroalkyl and SC₁₋₄fluoroalkyl. In some embodiments, A is selected from phenyl, C₃₋₁₀cycloalkyl, 3- to 6-membered heterocycloalkyl comprising 1 to 3 heteromoeities independently selected from O, S, S(O), SO₂, N and NR⁵⁴ and 5- to 6-membered heteroaryl comprising 1 to 3 heteromoeities independently selected from O, S, S(O), SO₂, N and NR⁵⁴, wherein the phenyl, C₃₋₁₀cycloalkyl, 3- to 6-membered heterocycloalkyl and 5- to 6-membered heteroaryl are optionally substituted with one or two substituents independently selected from F, Cl, Br, C₁₋₄alkyl, C₁₋₄deuteroalkyl, C₁₋₄fluoroalkyl, OC₁₋₄alkyl, OC₁₋₄deuteroalkyl, OC₁₋₄fluoroalkyl, N(R^{54a})(R^{54b}), C₁₋₄alkyleneN(R^{54a})(R^{54b}), SC₁₋₄alkyl, SC₁₋₄deuteroalkyl and SC₁₋₄fluoroalkyl. In some embodiments, A is selected from phenyl, C₃₋₆cycloalkyl, 5- to 6-membered heterocycloalkyl comprising 1 to 2 heteromoeities independently selected from O, S, S(O), SO₂, N and NR⁵⁴ and 5- to 6-membered heteroaryl comprising 1 to 3 heteromoeities independently selected from O, S, S(O), SO₂, N and NR⁵⁴, wherein the phenyl, C₃₋₁₀cycloalkyl, 3- to 6-membered heterocycloalkyl and 5- to 6-membered heteroaryl are optionally substituted with one or two substituents independently selected from F, Cl, C₁₋₄alkyl, C₁₋₄deuteroalkyl, C₁₋₄fluoroalkyl, OC₁₋₄alkyl, OC₁₋₄deuteroalkyl and OC₁₋₄fluoroalkyl.

In some embodiments, A is phenyl, optionally substituted with one or two substituents independently selected from F, Cl, C₁₋₄alkyl, C₁₋₄deuteroalkyl, C₁₋₄fluoroalkyl, OC₁₋₄alkyl, OC₁₋₄ deuteroalkyl, OC₁₋₄fluoroalkyl, N(R^{54a})(R^{54b}), C₁₋₄alkyleneN(R^{54a})(R^{54b}), SC₁₋₄alkyl, SC₁₋₄deuteroalkyl and SC₁₋₄fluoroalkyl. In some embodiments, A is phenyl, optionally substituted with one or two substituents independently selected from F, Cl, C₁₋₄alkyl, C₁₋₄deuteroalkyl, C₁₋₄fluoroalkyl, OC₁₋₄alkyl, OC₁₋₄deuteroalkyl and OC₁₋₄fluoroalkyl.

In some embodiments, A is C₃₋₆cycloalkyl optionally substituted with one or two substituents independently selected from F, Cl, C₁₋₄alkyl, C₁₋₄deuteroalkyl, C₁₋₄fluoroalkyl, OC₁₋₄ alkyl, OC₁₋₄deuteroalkyl, OC₁₋₄fluoroalkyl, N(R^{54a})(R^{54b}), C₁₋₄alkyleneN(R^{54a})(R^{54b}) SC₁₋₄alkyl, SC₁₋₄deuteroalkyl and SC₁₋₄fluoroalkyl. In some embodiments, A is C₃₋₆cycloalkyl optionally substituted with one or two substituents independently selected from F, Cl, C₁₋₄alkyl, C₁₋₄deuteroalkyl, C₁₋₄fluoroalkyl, OC₁₋₄alkyl, OC₁₋₄deuteroalkyl and OC₁₋₄fluoroalkyl. In some embodiments, the C₃₋₆cycloalkyl in A is selected from cyclopropyl, cyclobutyl, cyclopentyl and cyclohexyl. In some embodiments, the C₃₋₆cycloalkyl in A is selected from cyclopropyl, cyclobutyl and cyclopentyl. In some embodiment, the C₃₋₆cycloalkyl in A is cyclopropyl.

In some embodiments, A is 5- to 6-membered heterocycloalkyl comprising 1 to 2 heteromoeities independently selected from O, S, S(O), SO₂, N and NR⁵⁴ optionally substituted with one or two substituents independently selected from F, Cl, C₁₋₄alkyl, C₁₋₄deuteroalkyl, C₁₋₄ fluoroalkyl, OC₁₋₄alkyl, OC₁₋₄deuteroalkyl, OC₁₋₄fluoroalkyl, N(R^{54a})(R^{54b}) C₁₋₄alkyleneN(R^{54a})(R^{54b}), SC₁₋₄alkyl, SC₁₋₄deuteroalkyl and SC₁₋₄fluoroalkyl. In some embodiments, A is 5- to 6-membered heterocycloalkyl comprising 1 to 2 heteromoeities independently selected from O, S, S(O), SO₂, N and NR⁵⁴ optionally substituted with one or two substituents independently selected from F, Cl, C₁₋₄alkyl, C₁₋₄deuteroalkyl, C₁₋₄fluoroalkyl, OC₁₋₄alkyl, OC₁₋₄deuteroalkyl and OC₁₋₄fluoroalkyl. In some embodiments, the 3- to 6-membered heterocycloalkyl comprising 1 to 2 heteromoeities independently selected from O, S, S(O), SO₂, N and NR⁵⁴ in A is selected from aziridinyl, oxiranyl, azetidinyl, oxetanyl, pyrrolidinyl, imidazolidinyl, imidazolinyl, pyrazolinyl, tetrahydrofuranyl, piperidinyl, piperazinyl, morpholinyl, tetrahydropyranyl, thianyl, thianyl oxide and thianyl dioxide. In some embodiments, the 5- to 6-membered heterocycloalkyl comprising 1 to 2 heteromoeities independently selected from O, S, S(O), SO₂, N and NR⁵⁴ in A is selected from piperidinyl, piperazinyl, morpholinyl, tetrahydropyranyl, thianyl, and thianyl dioxide. In some embodiments, the 5- to 6-membered heterocycloalkyl comprising 1 to 2 heteromoeities independently selected from O, S, S(O), SO₂, N and NR⁵⁴ in A is selected from tetrahydropyranyl, thianyl, and thianyl dioxide.

In some embodiments, A is 5- to 6-membered heteroaryl comprising 1 to 3 heteromoeities independently selected from O, S, N and NR⁵⁴, optionally substituted with one or two substituents independently selected from F, Cl, C₁₋₄alkyl, C₁₋₄deuteroalkyl, C₁₋₄fluoroalkyl, OC₁₋₄alkyl, OC₁₋₄deuteroalkyl, OC₁₋₄fluoroalkyl, N(R^{54a})(R^{54b}) C₁₋₄alkyleneN(R^{54a})(R^{54b}), SC₁₋₄alkyl, SC₁₋₄deuteroalkyl and SC₁₋₄fluoroalkyl. In some embodiments, A is 5- to 6-membered heteroaryl comprising 1 to 3 heteromoeities independently selected from O, S, N and NR⁵⁴, optionally substituted with one or two substituents independently selected from F, Cl, C₁₋₄alkyl, C₁₋₄deuteroalkyl, C₁₋₄fluoroalkyl, OC₁₋₄alkyl, OC₁₋₄deuteroalkyl and OC₁₋₄fluoroalkyl. In some embodiments, the 5- to 6-membered heteroaryl comprising 1 to 3 heteromoeities independently selected from O, S, N and NR⁵⁴ in A is selected from pyrrolyl, furanyl, imidazolyl, oxazolyl, isoxazolyl, isothiazolyl, thiazoyl, pyrazolyl, thiophenyl, pyrazolyl, triazolyl, tetrazolyl, pyridinyl, pyrimidinyl, pyrazinyl and triazinyl. In some embodiments, the 5- to 6-membered heteroaryl comprising 1 to 3 heteromoeities independently selected from O, S, N and NR⁵⁴ in A is selected from pyrrolyl, imidazolyl, oxazolyl, isoxazolyl, isothiazolyl, thiazoyl, pyrazolyl, thiophenyl, pyrazolyl and pyridinyl. In some embodiments, the 5- to 6-membered heteroaryl is optionally substituted with one or two substituents independently selected from F, OC₁₋₄alkyl, OC₁₋₄deuteroalkyl and OC₁₋₄fluoroalkyl.

In some embodiments, R^{54a} and R^{54b} are independently selected from H and C₁₋₄alkyl, wherein all available hydrogen atoms are optionally and independently substituted with a fluorine atom or deuterium atom. In some embodiments, R^{54a} and R^{54b} are independently selected from H, D, C₁₋₄alkyl, C₁₋₄fluoroalkyl and C₁₋₄deuteroalkyl. In some embodiments, R^{54a} and R^{54b} are independently selected from H, D, CH₃, CD₂H, CDH₂, CD₃, CF₃, CHF₂, CH₂CH₃, CH(CH₃)₂, CH₂CH₂D, CH₂CD₂H and CD₂CD₃. In some embodiments, R^{54a} and R^{54b} are independently selected from H, CH₃, CD₃, CF₂H and CF₃. In some embodiments, R^{54a} and R^{54b} are independently selected from H, CH₃ and CD₃.

In some embodiments, R⁵⁴, R⁵⁵, R⁵⁶, R⁵⁷ and R⁵⁸ are independently selected from H, D, C₁₋₄alkyl, C₁₋₄fluoroalkyl and C₁₋₄deuteroalkyl. In some embodiments, R⁵⁴, R⁵⁵, R⁵⁶, R⁵⁷ and R⁵⁸ are independently selected from H, D, F, CH₃, CD₂H, CDH₂, CD₃, CF₃ and CHF₂. In some embodiments, R⁵⁴, R⁵⁵, R⁵⁶, R⁵⁷ and R⁵⁸ are independently selected from H, D, CH₃, CF₃ and CD₃. In some embodiments, R⁵⁴, R⁵⁵, R⁵⁶, R⁵⁷ and R⁵⁸ are independently selected from CH₃ and CD₃.

In some embodiments, the compound of Formula I is defined as follows:

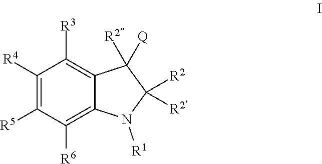

or a pharmaceutically acceptable salt, solvate and/or prodrug thereof,
wherein:
R¹ is H;
Q is Q3:

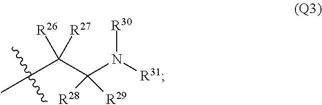

R², R^{2′}, R^{2″}, R³ and R⁶ are independently selected from H, D and F;
one or both of R⁴ and R⁵ is selected from H, C₁₋₄alkoxy, C₁₋₄fluoralkoxy and C₁₋₄deuteroalkoxy, or
R⁴ and R⁵ are linked together to form O—(CH₂)₁₋₂O;
R²⁶, R²⁷, R²⁸ and R²⁹ are independently selected from H and D; and
R³⁰ and R³¹ are independently selected from H, C₁₋₄alkyl, C₁₋₄fluoroalkyl and C₁₋₄deuteroalkyl;
provided that when R²⁶, R²⁷, R²⁸ and R²⁹ are all H and R³⁰ and R³¹ are H or CH₃, and:
R¹, R², R^{2′}, R^{2″}, R³, R⁴, R⁵ and R⁶ are all H,
R¹, R², R^{2′}, R^{2″}, R³, R⁵ and R⁶ are all H and R⁴ is OCH₃, or
R¹, R², R^{2′}, R^{2″}, R³, R⁴, R⁵ and R⁶ are all H and R⁵ is OCH₃,
then the compound of Formula I is an (R)- or (S)-enantiomer of the carbon to which Q is attached.

In some embodiments, the compound of Formula I is defined as follows:

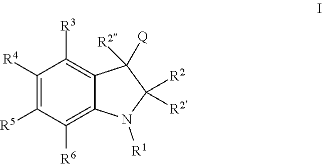

or a pharmaceutically acceptable salt, solvate and/or prodrug thereof,
wherein:
R¹ is H;
Q is selected from Q1, Q2, Q3, Q4 and Q5:

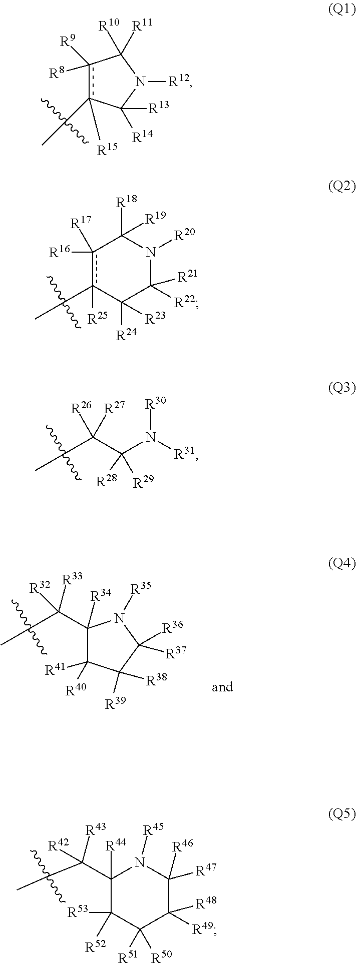

[symbol] is a single bond or a double bond provided that when [symbol] in Q1 is a double bond then R⁹ and R¹⁵ are not present, and when [symbol] in Q2 is a double bond then R¹⁷ and R²⁵ are not present;
R², R^{2′}, R^{2″}, R³ and R⁶ are independently selected from H, D and F;
one or both of R⁴ and R⁵ is selected from H, C₁₋₄alkoxy, C₁₋₄fluoralkoxy and C₁₋₄deuteroalkoxy, or
R⁴ and R⁵ are linked together to form O—(CH₂)₁₋₂O;
R⁸, R⁹, R¹⁰, R¹¹, R¹³, R¹⁴, R¹⁵, R¹⁶, R¹⁷, R¹⁸, R¹⁹, R²¹, R²², R²³, R²⁴, R²⁵, R²⁶, R²⁷, R²⁸, R²⁹, R³², R³³, R³⁴, R³⁶, R³⁷, R³⁸, R³⁹, R⁴⁰, R⁴¹, R⁴², R⁴³, R⁴⁴, R⁴⁶, R⁴⁷, R⁴⁸, R⁴⁹, R⁵⁰, R⁵¹, R⁵², R⁵⁴, R⁵⁵, R⁵⁶ and R⁵⁷ are independently selected from H and D; and
R¹², R²⁰, R³⁰, R³¹, R³⁵ and R⁴⁵ are independently selected from H, C₁₋₄alkyl, C₁₋₄fluoroalkyl and C₁₋₄deuteroalkyl;
provided that when Q is Q3, R²⁶, R²⁷, R²⁸ and R²⁹ are all H and R³⁰ and R³¹ are H or CH₃, and:
R¹, R², R^{2′}, R^{2″}, R³, R⁴, R⁵ and R⁶ are all H,
R¹, R², R^{2′}, R^{2″}, R³, R⁵ and R⁶ are all H and R⁴ is OCH₃, or
R¹, R², R^{2′}, R^{2″}, R³, R⁴, R⁵ and R⁶ are all H and R⁵ is OCH₃,
then the compound of Formula I is an (R)- or (S)-enantiomer of the carbon to which Q is attached.

In some embodiments, the compound of Formula I is defined as follows:

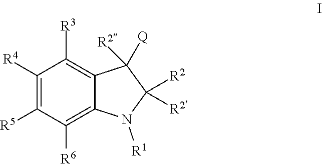

or a pharmaceutically acceptable salt, solvate and/or prodrug thereof,
wherein:
R¹ is H;
Q is Q3:

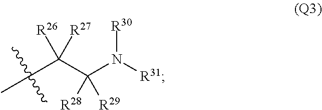

[symbol] over a bond means that the bond is attached to a remaining portion of the compound;
R², R^{2′} and R^{2″} are independently selected H, D and F;
R³, R⁴, R⁵ and R⁶ are independently selected H, C₁₋₄alkoxy, C₁₋₄fluoralkoxy and C₁₋₄deuteroalkoxy, or
two adjacent of R³, R⁴, R⁵ and R⁶ are linked together to form O—(CH₂)₁₋₂O and the remaining of R³, R⁴, R⁵ and R⁶ are independently selected from H, halo, C₁₋₆alkyl and C₁₋₆alkoxy;
R²⁶, R²⁷, R²⁸ and R²⁹ are independently selected from H, halo and C₁₋₆alkyl;
R³⁰ and R³¹ are independently selected from H, C₁₋₆alkyl and C(O)C₁₋₆alkyl, or
R³⁰ and R³¹, together with the N atom to which they are bound, form a 3- to 6-membered heterocyclic ring which optionally comprises one or two additional heteromoieties independently selected from O, S, S(O), SO₂, N, and NR⁵⁸;
R⁵⁸ is independently selected from H and C₁₋₆alkyl; and
all available hydrogen atoms are optionally and independently substituted with a fluorine atom or chlorine atom and all available atoms are optionally substituted with alternate isotope thereof,
provided that when R²⁶, R²⁷, R²⁸ and R²⁹ are all H and R³⁰ and R³¹ are H or CH₃, and:
R¹, R², R^{2′}, R^{2″}, R³, R⁴, R⁵ and R⁶ are all H,
R¹, R², R^{2′}, R^{2″}, R³, R⁵ and R⁶ are all H and R⁴ is OCH₃, or
R¹, R², R^{2′}, R^{2″}, R³, R⁴, R⁵ and R⁶ are all H and R⁵ is OCH₃,
then the compound of Formula I is an (R)- or (S)-enantiomer of the carbon to which Q is attached.

In some embodiments, the compound of Formula I is defined as follows:

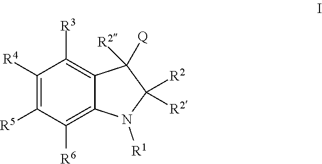

or a pharmaceutically acceptable salt, solvate and/or prodrug thereof,
wherein:
R¹ is selected from C(O)R⁷, CO₂R⁷, C(O)N(R⁷)(R⁷), S(O)R⁷, SO₂R⁷, C₁₋₆alkyleneR⁷ and R⁷;
Q is Q3:

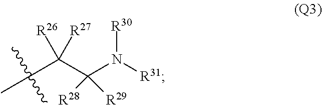

[symbol] over a bond means that the bond is attached to a remaining portion of the compound;
R², R^{2′} and R^{2″} are independently selected from H, halo, C₁₋₆alkyl, C₁₋₆alkoxy, C₁₋₁₀ alkyleneN(R^{2a})(R^{2b}) and SC₁₋₆alkyl;
R^{2a} and R^{2b} are independently selected from H and C₁₋₆alkyl;
R³ and R⁶ are independently selected from H, halo, N(R^{5′})(R^{6′}), C₁₋₆alkyl, C₁₋₆alkoxy, C₁₋₁₀ alkyleneN(R^{5′})(R^{6′}) and SC₁₋₆alkyl, the latter four groups being optionally substituted with one or two substituents selected from OH and C₁₋₆alkoxy;
R⁴ and R⁵ are independently selected from H, halo, N(R^{5′})(R^{6′}), C₁₋₆alkyl, C₁₋₆alkoxy, C₁₋₆alkyleneN(R^{5′})(R^{6′}) and SC₁₋₆alkyl, the latter four groups being optionally substituted with one or two substituents selected from OH and C₁₋₆alkoxy; or
R⁴ and R⁵ are linked together to form O—(CH₂)₁₋₂O; or
one of R⁴ and R⁵ is selected from A, O-A and C₁₋₄alkyleneA and the other of R⁴ and R⁵ is H or halo;
R^{5′} and R^{6′} are independently selected from H and C₁₋₆alkyl; A is selected from phenyl, C₃₋₆ cycloalkyl, 3- to 6-membered heterocycloalkyl comprising 1 to 4 heteromoeities independently selected from O, S, S(O), SO₂, N and NR⁵⁴ and 5- to 6-membered heteroaryl comprising 1 to 4 heteromoeities independently selected from O, S, S(O), SO₂, N and NR⁵⁴, wherein the phenyl, C₃₋₁₀cycloalkyl, 3- to 6-membered heterocycloalkyl and 5- to 6-membered heteroaryl are optionally substituted with one or more substituents independently selected from halo, C₁₋₄alkyl, OC₁₋₄alkyl, N(R^{54a})(R^{54b}), C₁₋₄alkyleneN(R^{54a})(R^{54b}) and SC₁₋₄alkyl;
R⁷ is selected from H, C₁₋₆alkyl, C₂₋₆alkenyl, C₂₋₆alkynyl, phenyl, C₃₋₆cycloalkyl, 3- to 6-membered heterocycloalkyl comprising 1 to 4 heteromoeities independently selected from O, S, N and NR^{7″}, and 5- to 6-membered heteroaryl comprising 1 to 4 heteromoeities independently selected from O, S, N and NR^{7″}, wherein the latter 5 groups are optionally substituted with one or more substituents independently selected from halo, OR⁵⁵, N(R⁵⁵)(R⁵⁶) and SR⁵⁵ and/or the C₁₋₆alkyl is optionally interrupted by one to three heteromoieties independently selected from O, C(O), CO₂ and NR⁵⁷;
R^{7′} is selected from H and C₁₋₆alkyl;
R^{7″} is selected from H and C₁₋₆alkyl;
R²⁶, R²⁷, R²⁸ and R²⁹ are independently selected from H, halo and C₁₋₆alkyl;
R³⁰ and R³¹ are independently selected from H, C₁₋₆alkyl and C(O)C₁₋₆alkyl, or
R³⁰ and R³¹, together with the N atom to which they are bound, form a 3- to 6-membered heterocyclic ring which optionally comprises one or two additional heteromoieties independently selected from O, S, S(O), SO₂, N, and NR⁵⁸;
R⁵⁴, R⁵⁵, R⁵⁶, R⁵⁷ and R⁵⁸ are independently selected from H and C₁₋₆alkyl;
R^{54a} and R^{54b} are independently selected from H and C₁₋₆alkyl; and all available hydrogen atoms are optionally and independently substituted with a fluorine atom or chlorine atom and all available atoms are optionally substituted with alternate isotope thereof,
provided that when R²⁶, R²⁷, R²⁸ and R²⁹ are all H and R³⁰ and R³¹ are H or CH₃, and:
R¹ is R⁷, R⁷ is H and R², R^{2′}, R^{2″}, R³, R⁴, R⁵ and R⁶ are all H,
R¹ is R⁷, R⁷ is H, R², R^{2′}, R^{2″}, R³, R⁵ and R⁶ are all H and R⁴ is OCH₃, or
R¹ is R⁷, R⁷ is H, R², R^{2′}, R^{2″}, R³, R⁴, R⁵ and R⁶ are all H and R⁵ is OCH₃,
then the compound of Formula I is an (R)- or (S)-enantiomer of the carbon to which Q is attached.

In some embodiments, the compound of Formula I is defined as follows:

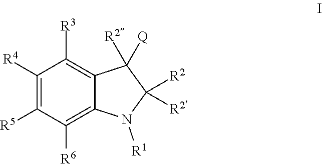

or a pharmaceutically acceptable salt, solvate and/or prodrug thereof,
wherein:
R¹ is H;
Q is Q3:

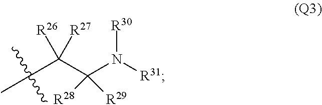

[symbol] over a bond means that the bond is attached to a remaining portion of the compound;
R², R^{2′} and R^{2″} are independently selected from H, halo, C₁₋₆alkyl, C₁₋₆alkoxy, C₁₋₆ alkyleneN(R^{2a})(R^{2b}) and SC₁₋₆alkyl;
R^{2a} and R^{2b} are independently selected from H and C₁₋₆alkyl;
R³, R⁴, R⁵ and R⁶ are independently selected from H, halo, N(R^{5′})(R^{6′}), C₁₋₆alkyl, C₁₋₆alkoxy, C₁₋₆alkyleneN(R^{5′})(R^{6′}) and SC₁₋₆alkyl, the latter two groups being optionally substituted with one or two substituents selected from OH and C₁₋₆alkoxy, or
two adjacent R³, R⁴, R⁵ and R⁶ are linked together to form O—(CH₂)₁₋₂O and the remaining of R³, R⁴, R⁵ and R⁶ are independently selected from H, halo, C₁₋₆alkyl, C₁₋₆alkoxy N(R^{5′})(R^{6′}), C₁₋₆alkyleneN(R^{5′})(R^{6′}) and SC₁₋₆alkyl;
R^{5′} and R^{6′} are independently selected from H and C₁₋₆alkyl;
R²⁶, R²⁷, R²⁸ and R²⁹ are independently selected from H, halo and C₁₋₆alkyl;
R³⁰ and R³¹ are independently selected from H, C₁₋₆alkyl and C(O)C₁₋₆alkyl, or
R³⁰ and R³¹, together with the N atom to which they are bound, form a 3- to 6-membered heterocyclic ring which optionally comprises one or two additional heteromoieties independently selected from O, S, S(O), SO₂, N, and NR⁵⁸;
R⁵⁸ is selected from H and C₁₋₆alkyl; and
all available hydrogen atoms are optionally and independently substituted with a fluorine atom or chlorine atom and all available atoms are optionally substituted with alternate isotope thereof,
provided that when R²⁶, R²⁷, R²⁸ and R²⁹ are all H and R³⁰ and R³¹ are H or CH₃, and:
R¹, R², R^{2′}, R^{2″}, R³, R⁴, R⁵ and R⁶ are all H,
R¹, R², R^{2′}, R^{2″}, R³, R⁵ and R⁶ are all H and R⁴ is OCH₃, or
R¹, R², R^{2′}, R^{2″}, R³, R⁴, R⁵ and R⁶ are all H and R⁵ is OCH₃,
then the compound of Formula I is an (R)- or (S)-enantiomer of the carbon to which Q is attached.

In some embodiments, the compound of Formula I is defined as follows:

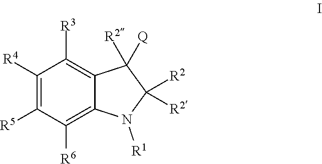

or a pharmaceutically acceptable salt, solvate and/or prodrug thereof,
wherein:
R¹ is selected from C(O)R⁷, CO₂R⁷, C(O)N(R⁷)(R^{7′}), S(O)R⁷, SO₂R⁷, C₁₋₆alkyleneR⁷ and R⁷;
Q is Q3:

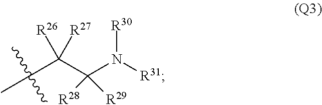

[symbol] over a bond means that the bond is attached to a remaining portion of the compound;
R², R^{2′} and R^{2″} are independently selected from H, halo, C₁₋₆alkyl, C₁₋₆alkoxy, C₁₋₆ alkyleneN(R^{2a})(R^{2b}) and SC₁₋₆alkyl;
R^{2a} and R^{2b} are independently selected from H and C₁₋₆alkyl;
one of R³, R⁴, R⁵ and R⁶ is selected from A, O-A and C₁₋₄alkyleneA and the remaining of R³, R⁴, R⁵ and R⁶ are independently H or halo;
A is selected from phenyl, C₃₋₆cycloalkyl, 3- to 6-membered heterocycloalkyl comprising 1 to 4 heteromoeities independently selected from O, S, S(O), SO₂, N and NR⁵⁴ and 5- to 6-membered heteroaryl comprising 1 to 4 heteromoeities independently selected from O, S, S(O), SO₂, N and NR⁵⁴, wherein the phenyl, C₃₋₁₀cycloalkyl, 3- to 6-membered heterocycloalkyl and 5- to 6-membered heteroaryl are optionally substituted with one or more substituents independently selected from halo, C₁₋₄alkyl,OC₁₋₄alkyl, N(R^{54a})(R^{54b}), C₁₋₄ alkyleneN(R^{54a})(R^{54b}) and SC₁₋₄alkyl;
R⁷ is selected from H, C₁₋₆alkyl, C₂₋₆alkenyl, C₂₋₆alkynyl, phenyl, C₃₋₆cycloalkyl, 3- to 6-membered heterocycloalkyl comprising 1 to 4 heteromoeities independently selected from O, S, N and NR^{7″}, and 5- to 6-membered heteroaryl comprising 1 to 4 heteromoeities independently selected from O, S, N and NR^{7″}, wherein the latter 5 groups are optionally substituted with one or more substituents independently selected from halo, OR⁵⁵, N(R⁵⁵)(R⁵⁶) and SR⁵⁵ and/or the C₁₋₆alkyl is optionally interrupted by one to three heteromoieties independently selected from O, C(O), CO₂ and NR⁵⁷;
R^{7′} is selected from H and C₁₋₆alkyl;
R^{7″} is selected from H and C₁₋₆alkyl;
R²⁶, R²⁷, R²⁸ and R²⁹ are independently selected from H, halo and C₁₋₆alkyl;
R³⁰ and R³¹ are independently selected from H, C₁₋₆alkyl and C(O)C₁₋₆alkyl, or
R³⁰ and R³¹, together with the N atom to which they are bound, form a 3- to 6-membered heterocyclic ring which optionally comprises one or two additional heteromoieties independently selected from O, S, S(O), SO₂, N, and NR⁵⁸;
R⁵⁴, R⁵⁵, R⁵⁶, R⁵⁷ and R⁵⁸ are independently selected from H and C₁₋₆alkyl;
R^{54a} and R^{54b} are independently selected from H and C₁₋₆alkyl; and
all available hydrogen atoms are optionally and independently substituted with a fluorine atom or chlorine atom and all available atoms are optionally substituted with alternate isotope thereof,
provided that when R²⁶, R²⁷, R²⁸ and R²⁹ are all H and R³⁰ and R³¹ are H or CH₃, and:
R¹ is R⁷, R⁷ is H and R², R^{2′}, R^{2″}, R³, R⁴, R⁵ and R⁶ are all H,
R¹ is R⁷, R⁷ is H, R², R^{2′}, R^{2″}, R³, R⁵ and R⁶ are all H and R⁴ is OCH₃, or
R¹ is R⁷, R⁷ is H, R², R^{2′}, R^{2″}, R³, R⁴, R⁵ and R⁶ are all H and R⁵ is OCH₃,
then the compound of Formula I is an (R)- or (S)-enantiomer of the carbon to which Q is attached.

A person skilled in the art would appreciate that the carbon to which Q is bonded is chiral. Therefore, in some embodiments the carbon to which Q is bonded is racemic. In some embodiments, the stereochemistry at the carbon to which Q is bonded is R. In some embodiments, the stereochemistry at the carbon to which Q is bonded is S. Therefore in some embodiments the compound of Formula I can have one of the following structures:

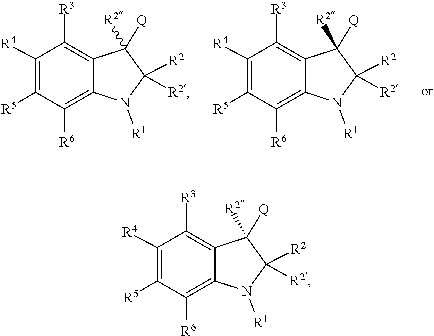

or a mixture thereof.

A person skilled in the art would further appreciate that the carbon to which R² and R^{2′} are bonded, when R² and R^{2′} are different, is also chiral. Therefore the present application also includes all stereoisomers at this carbon center and mixtures thereof.

In some embodiments, the compounds of Formula I are selected from the compounds listed in Table A below, or a pharmaceutically acceptable salt, solvate and/or prodrug thereof:

TABLE A

| Compound | |
|---|---|
| I.D. | Structure |
| (R)-I-1 | 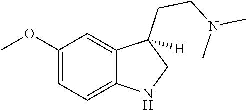 |
| (S)-I-1 | 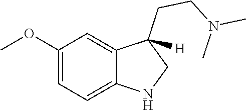 |
| I-2 | 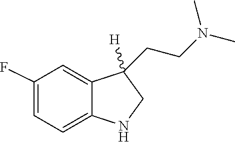 |
| I-3 | 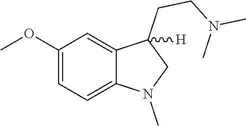 |
| (S)-I-3 | 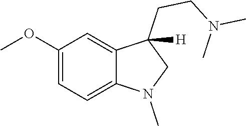 |
| (R)-I-3 | 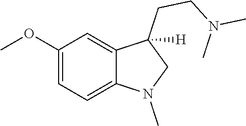 |
| (S)-I-4 | 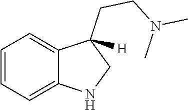 |
| (R)-I-4 | 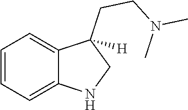 |
| I-6 | 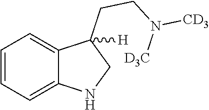 [SMILES: [2H]C([2H])([2H])N(CCC1CNc2ccccc21)C([2H])([2H])[2H]] |
| I-7 | 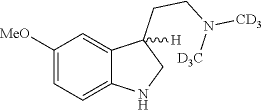 [SMILES: [2H]C([2H])([2H])N(CCC1CNc2ccc(OC)cc21)C([2H])([2H])[2H]] |
| I-9 | 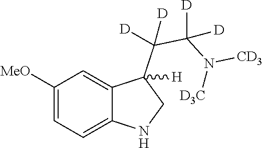 [SMILES: [2H]C([2H])([2H])N(C([2H])([2H])[2H])C([2H])([2H])C([2H])([2H])C1CNc2ccc(OC)cc21] |
| I-10 | 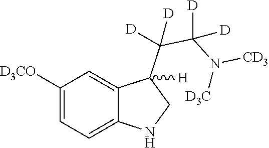 |
| I-11 | 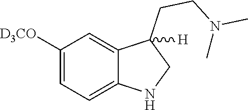 |
| I-12 | 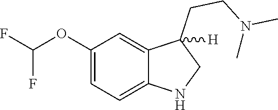 [SMILES: CN(C)CCC1CNc2ccc(OC(F)F)cc21] |
| I-13 | 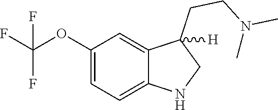 [SMILES: CN(C)CCC1CNc2ccc(OC(F)(F)F)cc21] |
| I-14 | 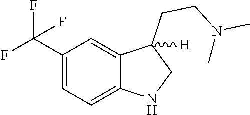 [SMILES: CN(C)CCC1CNc2ccc(C(F)(F)F)cc21] |
| I-15 | 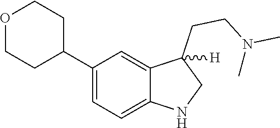 [SMILES: CN(C)CCC1CNc2ccc(C3CCOCC3)cc21] |
| I-16 | 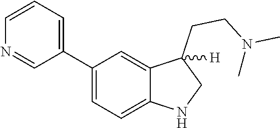 [SMILES: CN(C)CCC1CNc2ccc(-c3cccnc3)cc21] |
| I-17 | 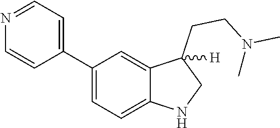 [SMILES: CN(C)CCC1CNc2ccc(-c3ccncc3)cc21] |
| I-18 | 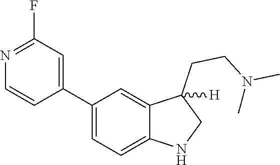 [SMILES: CN(C)CCC1CNc2ccc(-c3ccnc(F)c3)cc21] |
| I-19 | 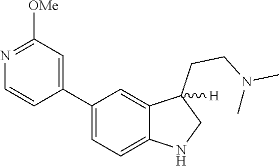 [SMILES: COc1cc(-c2ccc3c(c2)C(CCN(C)C)CN3)ccn1] |
| I-20 | 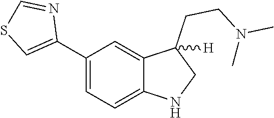 [SMILES: CN(C)CCC1CNc2ccc(-c3cscn3)cc21] |
| I-21 | 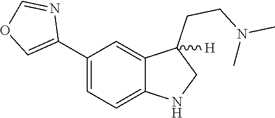 [SMILES: CN(C)CCC1CNc2ccc(-c3cocn3)cc21] |
| I-22 | 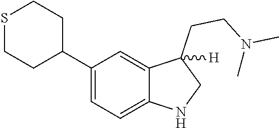 [SMILES: CN(C)CCC1CNc2ccc(C3CCSCC3)cc21] |
| I-23 | 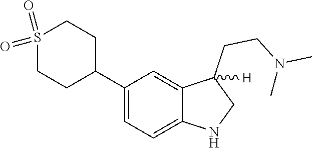 [SMILES: CN(C)CCC1CNc2ccc(C3CCS(=O)(=O)CC3)cc21] |
| I-24 | 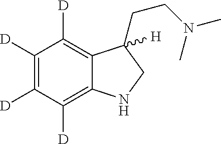 [SMILES: [2H]c1c([2H])c([2H])c2c(c1[2H])NCC2CCN(C)C] |
| I-25 | 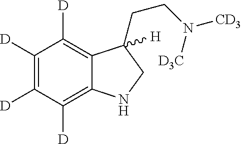 [SMILES: [2H]c1c([2H])c([2H])c2c(c1[2H])NCC2CCN(C([2H])([2H])[2H])C([2H])([2H])[2H]] |
| I-26 | 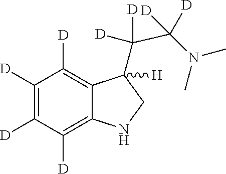 [SMILES: [2H]c1c([2H])c([2H])c2c(c1[2H])NCC2C([2H])([2H])C([2H])([2H])N(C)C] |
| I-27 | 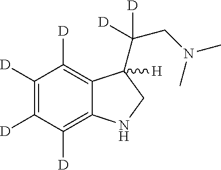 [SMILES: [2H]c1c([2H])c([2H])c2c(c1[2H])NCC2C([2H])([2H])CN(C)C] |
| I-28 | 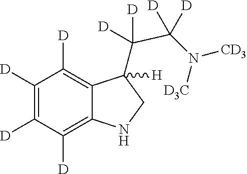 [SMILES: [2H]c1c([2H])c([2H])c2c(c1[2H])NCC2C([2H])([2H])C([2H])([2H])N(C([2H])([2H])[2H])C([2H])([2H])[2H]] |
| I-30 | 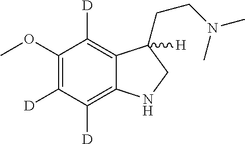 |
| I-31 | 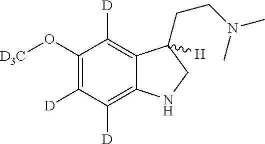 [SMILES: [2H]c1c([2H])c(OC([2H])([2H])[2H])c([2H])c2c1NCC2CCN(C)C] |
| I-32 | 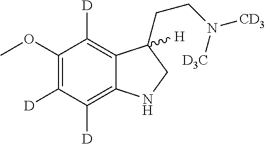 [SMILES: [2H]c1c([2H])c(OC)c([2H])c2c1NCC2CCN(C([2H])([2H])[2H])C([2H])([2H])[2H]] |
| I-33 | 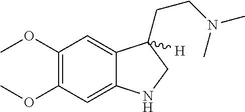 |
| (S)-I-33 | 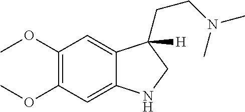 |
| (R)-I-33 | 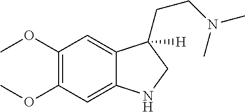 |
| I-36 | 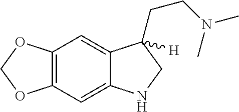 [SMILES: CN(C)CCC1CNc2cc3c(cc21)OCO3] |
| (S)-I-36 | 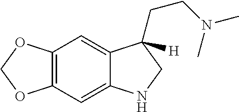 [SMILES: CN(C)CC[C@@H]1CNc2cc3c(cc21)OCO3] |
| (R)-I-36 | 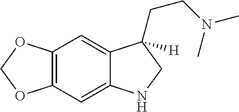 [SMILES: CN(C)CC[C@H]1CNc2cc3c(cc21)OCO3] |
| I-39 | 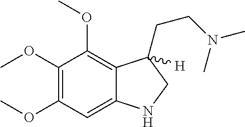 |
| I-40 | 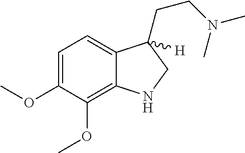 |
| I-41 | 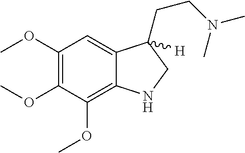 |
| I-43 | 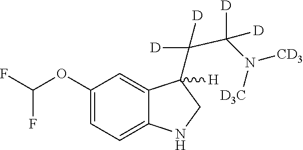 [SMILES: [2H]C([2H])([2H])N(C([2H])([2H])[2H])C([2H])([2H])C([2H])([2H])C1CNc2ccc(OC(F)F)cc21] |
| I-44 | 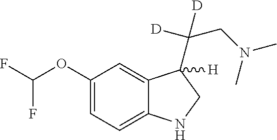 [SMILES: [2H]C([2H])(CN(C)C)C1CNc2ccc(OC(F)F)cc21] |
| I-45 | 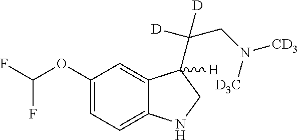 [SMILES: [2H]C([2H])(CN(C([2H])([2H])[2H])C([2H])([2H])[2H])C1CNc2ccc(OC(F)F)cc21] |
| I-107 | 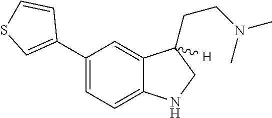 [SMILES: CN(C)CCC1CNc2ccc(-c3ccsc3)cc21] |
| (S)-I-107 | 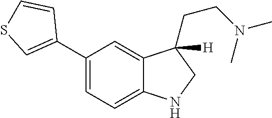 [SMILES: CN(C)CC[C@@H]1CNc2ccc(-c3ccsc3)cc21] |
| (R)-I-107 | 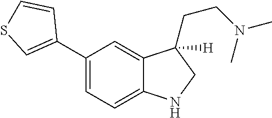 [SMILES: CN(C)CC[C@H]1CNc2ccc(-c3ccsc3)cc21] |
| (S)-I-108 | 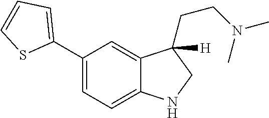 [SMILES: CN(C)CC[C@@H]1CNc2ccc(-c3cccs3)cc21] |
| I-109 | 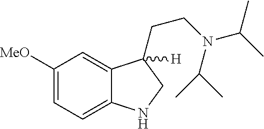 [SMILES: COc1ccc2c(c1)C(CCN(C(C)C)C(C)C)CN2] |
| I-110 | 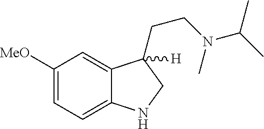 [SMILES: COc1ccc2c(c1)C(CCN(C)C(C)C)CN2] |
| I-111 | 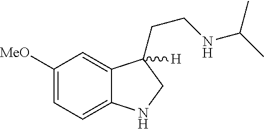 [SMILES: COc1ccc2c(c1)C(CCNC(C)C)CN2] |
| I-112 | 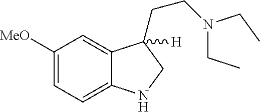 [SMILES: CCN(CC)CCC1CNc2ccc(OC)cc21] |
| I-113 | 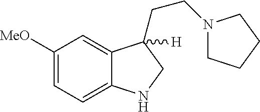 [SMILES: COc1ccc2c(c1)C(CCN1CCCC1)CN2] |
| I-114 | 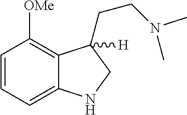 [SMILES: COc1cccc2c1C(CCN(C)C)CN2] |
| (S)-I-114 | 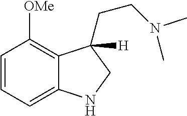 [SMILES: COc1cccc2c1[C@H](CCN(C)C)CN2] |
| (R)-I-114 | 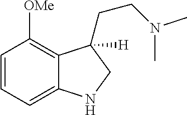 [SMILES: COc1cccc2c1[C@@H](CCN(C)C)CN2] |
| I-115 | 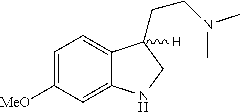 [SMILES: COc1ccc2c(c1)NCC2CCN(C)C] |
| (S)-I-115 | 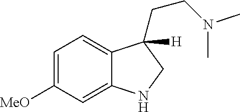 [SMILES: COc1ccc2c(c1)NC[C@H]2CCN(C)C] |
| (R)-I-115 | 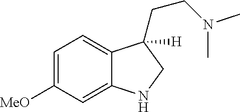 [SMILES: COc1ccc2c(c1)NC[C@@H]2CCN(C)C] |
| I-116 | 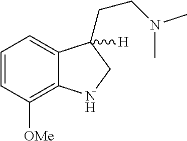 [SMILES: COc1cccc2c1NCC2CCN(C)C] |
| (S)-I-116 | 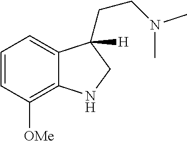 [SMILES: COc1cccc2c1NC[C@H]2CCN(C)C] |
| (R)-I-116 | 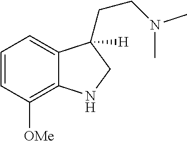 [SMILES: COc1cccc2c1NC[C@@H]2CCN(C)C] |
| I-117 | 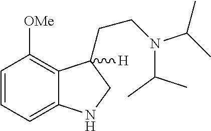 [SMILES: COc1cccc2c1C(CCN(C(C)C)C(C)C)CN2] |
| (S)-I-117 | 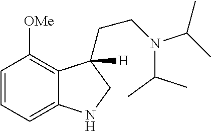 [SMILES: COc1cccc2c1[C@H](CCN(C(C)C)C(C)C)CN2] |
| (R)-I-117 | 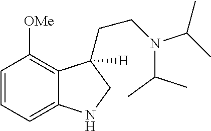 [SMILES: COc1cccc2c1[C@@H](CCN(C(C)C)C(C)C)CN2] |
| I-118 | 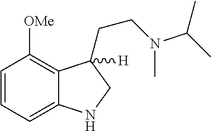 [SMILES: COc1cccc2c1C(CCN(C)C(C)C)CN2] |
| (S)-I-118 | 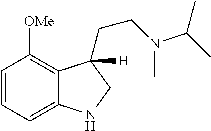 [SMILES: COc1cccc2c1[C@H](CCN(C)C(C)C)CN2] |
| (R)-I-118 | 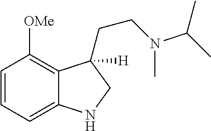 [SMILES: COc1cccc2c1[C@@H](CCN(C)C(C)C)CN2] |
| I-119 | 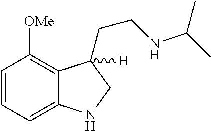 [SMILES: COc1cccc2c1C(CCNC(C)C)CN2] |
| (S)-I-119 | 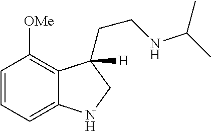 [SMILES: COc1cccc2c1[C@H](CCNC(C)C)CN2] |
| (R)-I-119 | 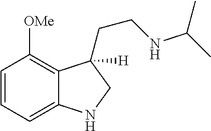 [SMILES: COc1cccc2c1[C@@H](CCNC(C)C)CN2] |
| I-120 | 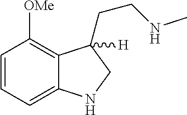 |
| (S)-I-120 | 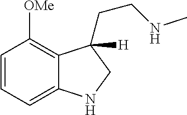 |
| (R)-I-120 | 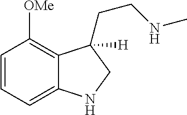 |
| I-121 | 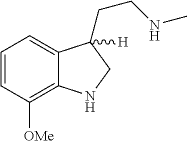 |
| (S)-I-121 | 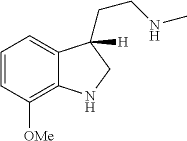 |
| (R)-I-121 | 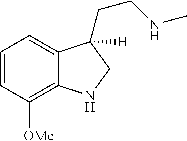 |
| I-122 | 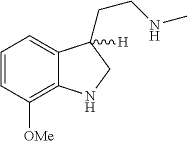 |
| (S)-I-122 | 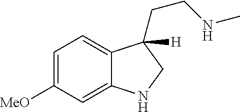 |
| (R)-I-122 | 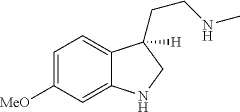 |
| I-123 | 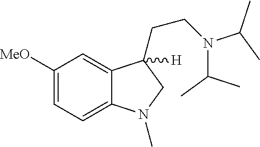 [SMILES: COc1ccc2c(c1)C(CCN(C(C)C)C(C)C)CN2C] |
| (S)-I-123 | 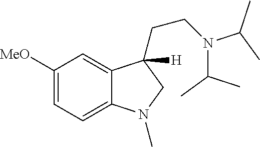 [SMILES: COc1ccc2c(c1)[C@H](CCN(C(C)C)C(C)C)CN2C] |
| (R)-I-123 | 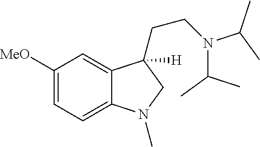 [SMILES: COc1ccc2c(c1)[C@@H](CCN(C(C)C)C(C)C)CN2C] |
| I-124 | 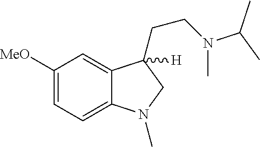 [SMILES: COc1ccc2c(c1)C(CCN(C)C(C)C)CN2C] |
| (S)-I-124 | 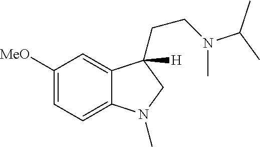 [SMILES: COc1ccc2c(c1)[C@H](CCN(C)C(C)C)CN2C] |
| (R)-I-124 | 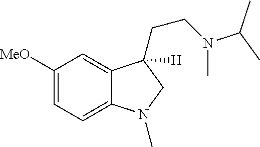 [SMILES: COc1ccc2c(c1)[C@@H](CCN(C)C(C)C)CN2C] |
| I-125 | 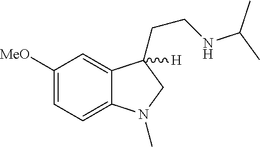 [SMILES: COc1ccc2c(c1)C(CCNC(C)C)CN2C] |
| (S)-I-125 | 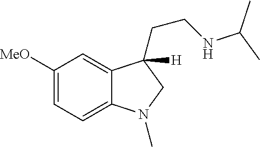 [SMILES: COc1ccc2c(c1)[C@H](CCNC(C)C)CN2C] |
| (R)-I-125 | 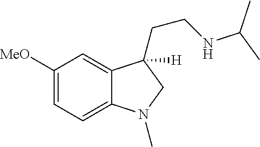 [SMILES: COc1ccc2c(c1)[C@@H](CCNC(C)C)CN2C] |
| I-126 | 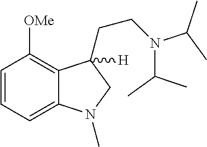 [SMILES: COc1cccc2c1C(CCN(C(C)C)C(C)C)CN2C] |
| (R)-I-126 | 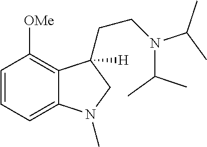 [SMILES: COc1cccc2c1[C@@H](CCN(C(C)C)C(C)C)CN2C] |
| (S)-I-126 | 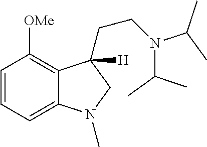 [SMILES: COc1cccc2c1[C@H](CCN(C(C)C)C(C)C)CN2C] |
| I-127 | 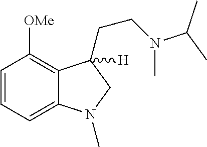 [SMILES: COc1cccc2c1C(CCN(C)C(C)C)CN2C] |
| (R)-I-127 | 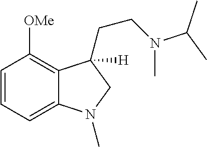 [SMILES: COc1cccc2c1[C@@H](CCN(C)C(C)C)CN2C] |
| (S)-I-127 | 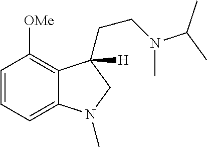 [SMILES: COc1cccc2c1[C@H](CCN(C)C(C)C)CN2C] |
| I-128 | 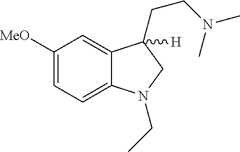 [SMILES: CCN1CC(CCN(C)C)c2cc(OC)ccc21] |
| (S)-I-128 | 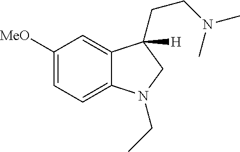 [SMILES: CCN1C[C@@H](CCN(C)C)c2cc(OC)ccc21] |
| (R)-I-128 | 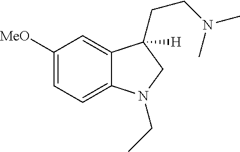 [SMILES: CCN1C[C@H](CCN(C)C)c2cc(OC)ccc21] |
| I-129 | 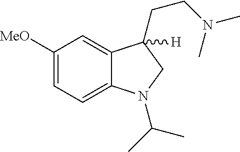 [SMILES: COc1ccc2c(c1)C(CCN(C)C)CN2C(C)C] |
| (S)-I-129 | 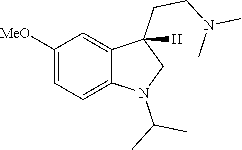 [SMILES: COc1ccc2c(c1)[C@H](CCN(C)C)CN2C(C)C] |
| (R)-I-129 | 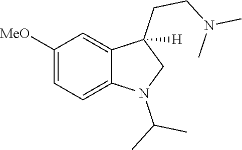 [SMILES: COc1ccc2c(c1)[C@@H](CCN(C)C)CN2C(C)C] |
| I-130 | 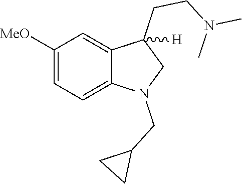 [SMILES: COc1ccc2c(c1)C(CCN(C)C)CN2CC1CC1] |
| I-131 | 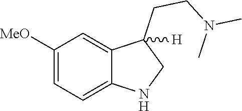 [SMILES: COc1ccc2c(c1)C(CCN(C)C)CN2] |
| I-132 | 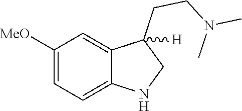 [SMILES: COc1ccc2c(c1)C(CCN(C)C)CN2] |
| I-133 | 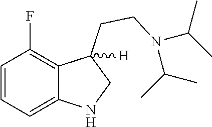 [SMILES: CC(C)N(CCC1CNc2cccc(F)c21)C(C)C] |
| (S)-I-133 | 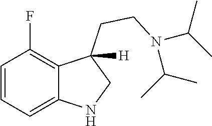 [SMILES: CC(C)N(CC[C@@H]1CNc2cccc(F)c21)C(C)C] |
| (R)-I-133 | 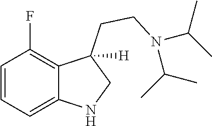 [SMILES: CC(C)N(CC[C@H]1CNc2cccc(F)c21)C(C)C] |
| I-134 | 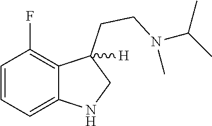 [SMILES: CC(C)N(C)CCC1CNc2cccc(F)c21] |
| (R)-I-134 | 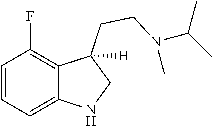 [SMILES: CC(C)N(C)CC[C@H]1CNc2cccc(F)c21] |
| (S)-I-134 | 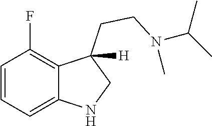 [SMILES: CC(C)N(C)CC[C@@H]1CNc2cccc(F)c21] |
| I-135 | 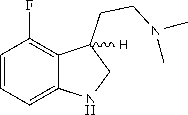 |
| (R)-I-135 | 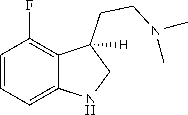 |
| (S)-I-135 | 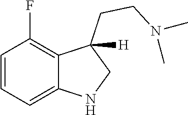 |
| I-136 | 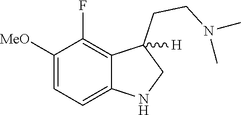 [SMILES: COc1ccc2c(c1F)C(CCN(C)C)CN2] |
| (S)-I-136 | 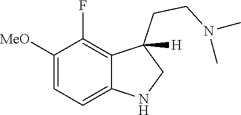 [SMILES: COc1ccc2c(c1F)[C@H](CCN(C)C)CN2] |
| (R)-I-136 | 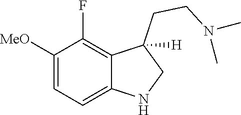 [SMILES: COc1ccc2c(c1F)[C@@H](CCN(C)C)CN2] |
| I-137 | 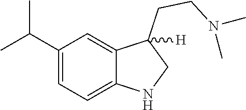 [SMILES: CC(C)c1ccc2c(c1)C(CCN(C)C)CN2] |
| (R)-I-137 | 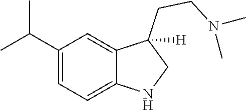 [SMILES: CC(C)c1ccc2c(c1)[C@@H](CCN(C)C)CN2] |
| (S)-I-137 | 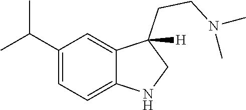 [SMILES: CC(C)c1ccc2c(c1)[C@H](CCN(C)C)CN2] |
| I-138 | 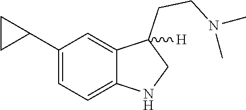 [SMILES: CN(C)CCC1CNc2ccc(C3CC3)cc21] |
| I-139 | 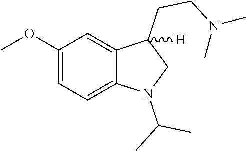 |
| I-140 | 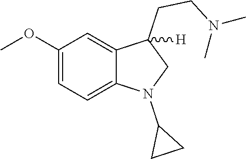 |
| I-141 | 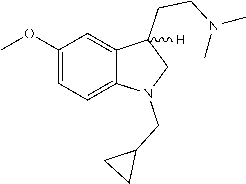 |
| I-142 | 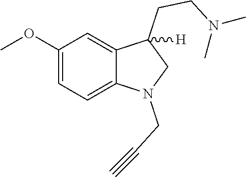 |
| I-143 | 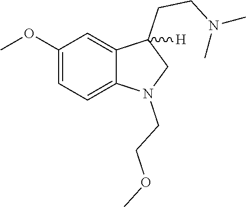 |
| I-144 | 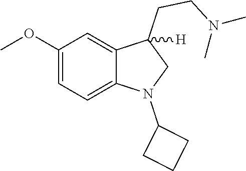 |
| I-145 | 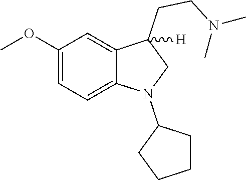 |
| I-146 | 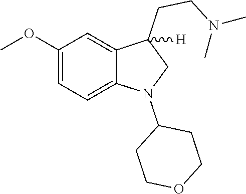 |
| I-147 | 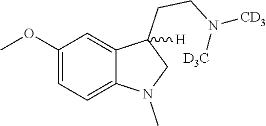 [SMILES: [2H]C([2H])([2H])N(CCC1CN(C)c2ccc(OC)cc21)C([2H])([2H])[2H]] |
| (R)-I-147 | 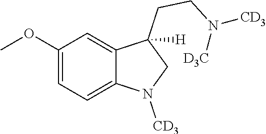 [SMILES: [2H]C([2H])([2H])N1C[C@H](CCN(C([2H])([2H])[2H])C([2H])([2H])[2H])c2cc(OC)ccc21] |
| | and |
| (S)-I-147 | 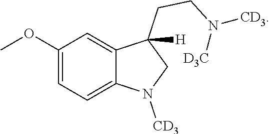 [SMILES: [2H]C([2H])([2H])N1C[C@@H](CCN(C([2H])([2H])[2H])C([2H])([2H])[2H])c2cc(OC)ccc21] |

In some embodiments, the compounds of Formula I are selected from the compounds listed below, or a pharmaceutically acceptable salt, solvate and/or prodrug thereof:

| Compound | |
|---|---|
| I.D. | Structure |
| (R)-I-1 | 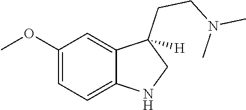 |
| (S)-I-1 | 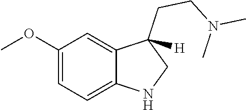 |
| I-2 | 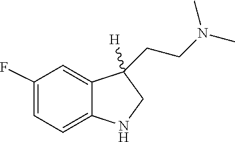 |
| I-3 | 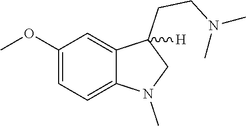 |
| (S)-I-4 | 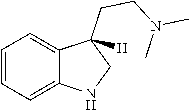 |
| (R)-I-4 | 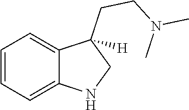 |
| I-6 | 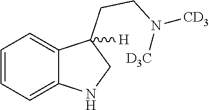 [SMILES: [2H]C([2H])([2H])N(CCC1CNc2ccccc21)C([2H])([2H])[2H]] |
| I-7 | 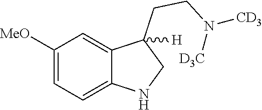 [SMILES: [2H]C([2H])([2H])N(CCC1CNc2ccc(OC)cc21)C([2H])([2H])[2H]] |
| I-9 | 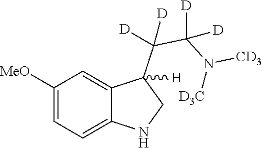 [SMILES: [2H]C([2H])([2H])N(C([2H])([2H])[2H])C([2H])([2H])C([2H])([2H])C1CNc2ccc(OC)cc21] |
| I-10 | 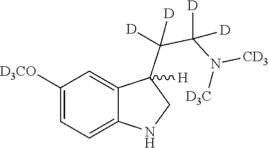 |
| I-11 | 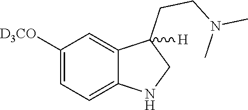 |
| I-12 | 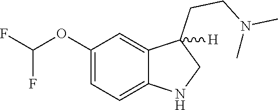 [SMILES: CN(C)CCC1CNc2ccc(OC(F)F)cc21] |
| I-13 | 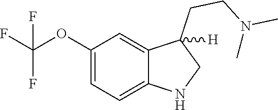 [SMILES: CN(C)CCC1CNc2ccc(OC(F)(F)F)cc21] |
| I-14 | 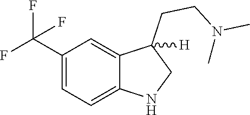 [SMILES: CN(C)CCC1CNc2ccc(C(F)(F)F)cc21] |
| I-15 | 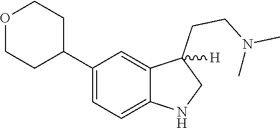 [SMILES: CN(C)CCC1CNc2ccc(C3CCOCC3)cc21] |
| I-16 | 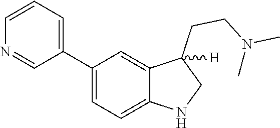 [SMILES: CN(C)CCC1CNc2ccc(-c3cccnc3)cc21] |
| I-17 | 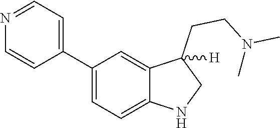 [SMILES: CN(C)CCC1CNc2ccc(-c3ccncc3)cc21] |
| I-18 | 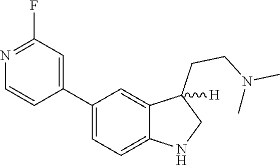 [SMILES: CN(C)CCC1CNc2ccc(-c3ccnc(F)c3)cc21] |
| I-19 | 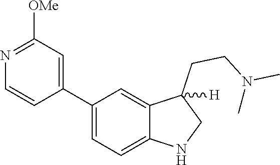 [SMILES: COc1cc(-c2ccc3c(c2)C(CCN(C)C)CN3)ccn1] |
| I-20 | 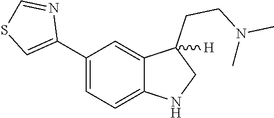 [SMILES: CN(C)CCC1CNc2ccc(-c3cscn3)cc21] |
| I-21 | 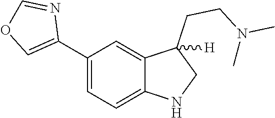 [SMILES: CN(C)CCC1CNc2ccc(-c3cocn3)cc21] |
| I-22 | 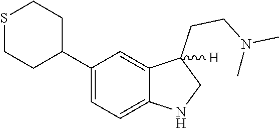 [SMILES: CN(C)CCC1CNc2ccc(C3CCSCC3)cc21] |
| I-23 | 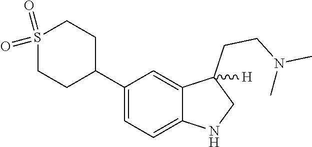 [SMILES: CN(C)CCC1CNc2ccc(C3CCS(=O)(=O)CC3)cc21] |
| I-24 | 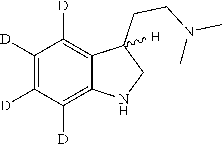 [SMILES: [2H]c1c([2H])c([2H])c2c(c1[2H])NCC2CCN(C)C] |
| I-25 | 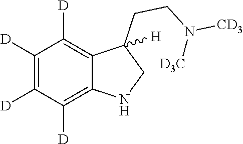 [SMILES: [2H]c1c([2H])c([2H])c2c(c1[2H])NCC2CCN(C([2H])([2H])[2H])C([2H])([2H])[2H]] |
| I-26 | 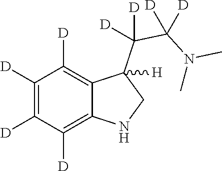 [SMILES: [2H]c1c([2H])c([2H])c2c(c1[2H])NCC2C([2H])([2H])C([2H])([2H])N(C)C] |
| I-27 | 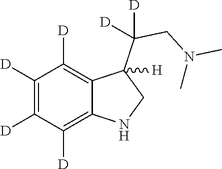 [SMILES: [2H]c1c([2H])c([2H])c2c(c1[2H])NCC2C([2H])([2H])CN(C)C] |
| I-28 | 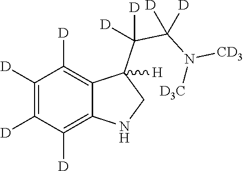 [SMILES: [2H]c1c([2H])c([2H])c2c(c1[2H])NCC2C([2H])([2H])C([2H])([2H])N(C([2H])([2H])[2H])C([2H])([2H])[2H]] |
| I-30 | 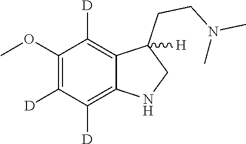 |
| I-31 | 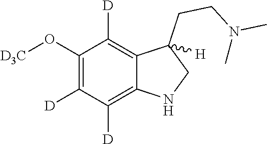 [SMILES: [2H]c1c([2H])c(OC([2H])([2H])[2H])c([2H])c2c1NCC2CCN(C)C] |
| I-32 | 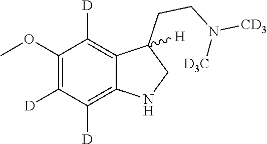 [SMILES: [2H]c1c([2H])c(OC)c([2H])c2c1NCC2CCN(C([2H])([2H])[2H])C([2H])([2H])[2H]] |
| I-33 | 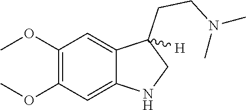 |
| (S)-I-33 | 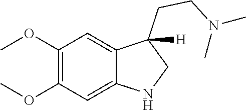 |
| (R)-I-33 | 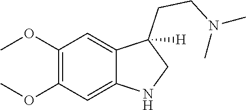 |
| I-36 | 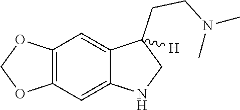 [SMILES: CN(C)CCC1CNc2cc3c(cc21)OCO3] |
| (S)-I-36 | 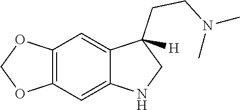 [SMILES: CN(C)CC[C@@H]1CNc2cc3c(cc21)OCO3] |
| (R)-I-36 | 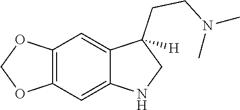 [SMILES: CN(C)CC[C@H]1CNc2cc3c(cc21)OCO3] |
| I-39 | 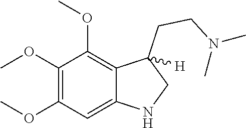 |
| I-40 | 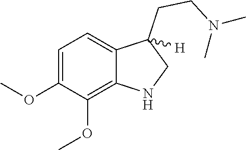 |
| I-41 | 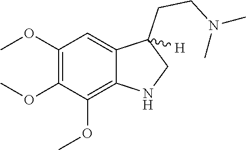 |
| I-43 | 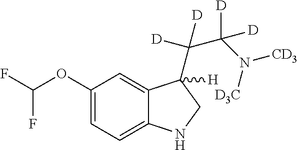 [SMILES: [2H]C([2H])([2H])N(C([2H])([2H])[2H])C([2H])([2H])C([2H])([2H])C1CNc2ccc(OC(F)F)cc21] |
| I-44 | 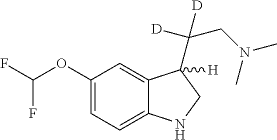 [SMILES: [2H]C([2H])(CN(C)C)C1CNc2ccc(OC(F)F)cc21] |
| I-45 | 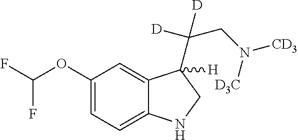 [SMILES: [2H]C([2H])(CN(C([2H])([2H])[2H])C([2H])([2H])[2H])C1CNc2ccc(OC(F)F)cc21] |

In some embodiments, the compound of Formula I is a zwitterion, and the present application includes a compound of Formula I and pharmaceutically acceptable salts, solvates, zwitterions and/or prodrugs thereof.

In some embodiments, the pharmaceutically acceptable salt is an acid addition salt or a base addition salt. The selection of a suitable salt may be made by a person skilled in the art. Suitable salts include acid addition salts that may, for example, be formed by mixing a solution of a compound with a solution of a pharmaceutically acceptable acid such as hydrochloric acid, sulfuric acid, acetic acid, trifluoroacetic acid, or benzoic acid. Additionally, acids that are generally considered suitable for the formation of pharmaceutically useful salts from basic pharmaceutical compounds are discussed, for example, by P. Stahl et al, Camille G. (eds.) and Handbook of Pharmaceutical Salts. Properties, Selection and Use. (2002) Zurich: Wiley VCH; S. Berge et al, Journal of Pharmaceutical Sciences 1977 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website).

An acid addition salt suitable for, or compatible with, the treatment of subjects is any non-toxic organic or inorganic acid addition salt of any basic compound. Basic compounds that form an acid addition salt include, for example, compounds comprising an amine group. Illustrative inorganic acids which form suitable salts include hydrochloric, hydrobromic, sulfuric, nitric and phosphoric acids, as well as acidic metal salts such as sodium monohydrogen orthophosphate and potassium hydrogen sulfate. Illustrative organic acids which form suitable salts include mono-, di- and tricarboxylic acids. Illustrative of such organic acids are, for example, acetic, trifluoroacetic, propionic, glycolic, lactic, pyruvic, malonic, succinic, glutaric, fumaric, malic, tartaric, citric, ascorbic, maleic, hydroxymaleic, benzoic, hydroxybenzoic, phenylacetic, cinnamic, mandelic, salicylic, 2-phenoxybenzoic, p-toluenesulfonic acid and other sulfonic acids such as methanesulfonic acid, ethanesulfonic acid and 2-hydroxyethanesulfonic acid. In some embodiments, exemplary acid addition salts also include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates (“mesylates”), naphthalenesulfonates, nitrates, oxalates, phosphates, propionates, salicylates, succinates, sulfates, tartarates, thiocyanates, toluenesulfonates (also known as tosylates) and the like. In some embodiments, the mono- or di-acid salts are formed and such salts exist in either a hydrated, solvated or substantially anhydrous form. In general, acid addition salts are more soluble in water and various hydrophilic organic solvents and generally demonstrate higher melting points in comparison to their free base forms. The selection criteria for the appropriate salt will be known to one skilled in the art. Other non-pharmaceutically acceptable salts such as but not limited to oxalates may be used, for example in the isolation of compounds of the application for laboratory use, or for subsequent conversion to a pharmaceutically acceptable acid addition salt.

A base addition salt suitable for, or compatible with, the treatment of subjects is any non-toxic organic or inorganic base addition salt of any acidic compound. Acidic compounds that form a basic addition salt include, for example, compounds comprising a carboxylic acid group. Illustrative inorganic bases which form suitable salts include lithium, sodium, potassium, calcium, magnesium or barium hydroxide as well as ammonia. Illustrative organic bases which form suitable salts include aliphatic, alicyclic or aromatic organic amines such as isopropylamine, methylamine, trimethylamine, picoline, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like. Exemplary organic bases are isopropylamine, diethylamine, ethanolamine, trimethylamine, dicyclohexylamine, choline and caffeine. The selection of the appropriate salt may be useful, for example, so that an ester functionality, if any, elsewhere in a compound is not hydrolyzed. The selection criteria for the appropriate salt will be known to one skilled in the art. In some embodiments, exemplary basic salts also include ammonium salts, alkali metal salts such as sodium, lithium and potassium salts, alkaline earth metal salts such as calcium and magnesium salts, salts with organic bases (for example, organic amines) such as dicyclohexylamine, Abutyl amine, choline and salts with amino acids such as arginine, lysine and the like. Basic nitrogen containing groups may be quarternized with agents such as lower alkyl halides (e.g., methyl, ethyl and butyl chlorides, bromides and iodides), dialkyl sulfates (e.g., dimethyl, diethyl and dibutyl sulfates), long chain halides (e.g., decyl, lauryl and stearyl chlorides, bromides and iodides), aralkyl halides (e.g., benzyl and phenethyl bromides) and others. Compounds carrying an acidic moiety can be mixed with suitable pharmaceutically acceptable salts to provide, for example, alkali metal salts (e.g., sodium or potassium salts), alkaline earth metal salts (e.g., calcium or magnesium salts) and salts formed with suitable organic ligands such as quaternary ammonium salts. Also, in the case of an acid (—COOH) or alcohol group being present, pharmaceutically acceptable esters can be employed to modify the solubility or hydrolysis characteristics of the compound.

All such acid salts and base salts are intended to be pharmaceutically acceptable salts within the scope of the application and all acid and base salts are considered equivalent to the free forms of the corresponding compounds for purposes of the application. In addition, when a compound of the application contains both a basic moiety, such as, but not limited to an aliphatic primary, secondary, tertiary or cyclic amine, an aromatic or heteroaryl amine, pyridine or imidazole and an acidic moiety, such as, but not limited to tetrazole or carboxylic acid, zwitterions (“inner salts”) may be formed and are included within the terms “salt(s)” as used herein. It is understood that certain compounds of the application may exist in zwitterionic form, having both anionic and cationic centers within the same compound and a net neutral charge. Such zwitterions are included within the application.

Solvates of compounds of the application include, for example, those made with solvents that are pharmaceutically acceptable. Examples of such solvents include water (resulting solvate is called a hydrate) and ethanol and the like. Suitable solvents are physiologically tolerable at the dosage administered.

Prodrugs of the compounds of the present application include, for example, conventional esters formed with available hydroxy, thiol, amino or carboxyl groups. Some common esters which have been utilized as prodrugs are phenyl esters, aliphatic (C₁-C₂₄) esters, acyloxymethyl esters, carbamates and amino acid esters.

It is understood and appreciated that in some embodiments, compounds of the present application may have at least one chiral center and therefore can exist as enantiomers and/or diastereomers. It is to be understood that all such isomers and mixtures thereof in any proportion are encompassed within the scope of the present application. It is to be further understood that while the stereochemistry of the compounds may be as shown in any given compound listed herein, such compounds may also contain certain amounts (for example, less than 20%, suitably less than 10%, more suitably less than 5%) of compounds of the present application having an alternate stereochemistry. It is intended that any optical isomers, as separated, pure or partially purified optical isomers or racemic mixtures thereof are included within the scope of the present application.

In some embodiments, the compounds of the present application can also include tautomeric forms, such as keto-enol tautomers and the like. Tautomeric forms can be in equilibrium or sterically locked into one form by appropriate substitution. It is intended that any tautomeric forms which the compounds form, as well as mixtures thereof, are included within the scope of the present application.

The compounds of the present application may further exist in varying amorphous and polymorphic forms and it is contemplated that any amorphous forms, polymorphs, or mixtures thereof, which form are included within the scope of the present application.

The compounds of the present application may further be radiolabeled and accordingly all radiolabeled versions of the compounds of the application are included within the scope of the present application. Therefore, the compounds of the application also include those in which one or more radioactive atoms are incorporated within their structure.

### III. Compositions

The compounds of the present application are suitably formulated in a conventional manner into compositions using one or more carriers. Accordingly, the present application also includes a composition comprising one or more compounds of the application and a carrier. The compounds of the application are suitably formulated into pharmaceutical compositions for administration to subjects in a biologically compatible form suitable for administration in vivo. Accordingly, the present application further includes a pharmaceutical composition comprising one or more compounds of the application and a pharmaceutically acceptable carrier. In embodiments of the application the pharmaceutical compositions are used in the treatment of any of the diseases, disorders or conditions described herein.

The compounds of the application are administered to a subject in a variety of forms depending on the selected route of administration, as will be understood by those skilled in the art. For example, a compound of the application is administered by oral, inhalation, parenteral, buccal, sublingual, insufflation, epidurally, nasal, rectal, vaginal, patch, pump, minipump, topical or transdermal administration and the pharmaceutical compositions formulated accordingly. In some embodiments, administration is by means of a pump for periodic or continuous delivery. Conventional procedures and ingredients for the selection and preparation of suitable compositions are described, for example, in Remington's Pharmaceutical Sciences (2000-20th edition) and in The United States Pharmacopeia: The National Formulary (USP 24 NF19) published in 1999.

Parenteral administration includes systemic delivery routes other than the gastrointestinal (GI) tract and includes, for example intravenous, intra-arterial, intraperitoneal, subcutaneous, intramuscular, transepithelial, nasal, intrapulmonary (for example, by use of an aerosol), intrathecal, rectal and topical (including the use of a patch or other transdermal delivery device) modes of administration. Parenteral administration may be by continuous infusion over a selected period of time.

In some embodiments, a compound of the application is orally administered, for example, with an inert diluent or with an assimilable edible carrier, or it is enclosed in hard or soft shell gelatin capsules, or it is compressed into tablets, or it is incorporated directly with the food of the diet. In some embodiments, the compound is incorporated with excipient and used in the form of ingestible tablets, buccal tablets, troches, capsules, caplets, pellets, granules, lozenges, chewing gum, powders, syrups, elixirs, wafers, aqueous solutions and suspensions and the like. In the case of tablets, carriers that are used include lactose, corn starch, sodium citrate and salts of phosphoric acid. Pharmaceutically acceptable excipients include binding agents (e.g., pregelatinized maize starch, polyvinylpyrrolidone or hydroxypropyl methylcellulose); fillers (e.g., lactose, microcrystalline cellulose or calcium phosphate); lubricants (e.g., magnesium stearate, talc or silica); disintegrants (e.g., potato starch or sodium starch glycolate); or wetting agents (e.g., sodium lauryl sulphate), or solvents (e.g. medium chain triglycerides, ethanol, water). In embodiments, the tablets are coated by methods well known in the art. In the case of tablets, capsules, caplets, pellets or granules for oral administration, pH sensitive enteric coatings, such as Eudragits™ designed to control the release of active ingredients are optionally used. Oral dosage forms also include modified release, for example immediate release and timed-release, formulations. Examples of modified-release formulations include, for example, sustained-release (SR), extended-release (ER, XR, or XL), time-release or timed-release, controlled-release (CR), or continuous-release (CR or Contin), employed, for example, in the form of a coated tablet, an osmotic delivery device, a coated capsule, a microencapsulated microsphere, an agglomerated particle, e.g., as of molecular sieving type particles, or, a fine hollow permeable fiber bundle, or chopped hollow permeable fibers, agglomerated or held in a fibrous packet. Timed-release compositions are formulated, for example as liposomes or those wherein the active compound is protected with differentially degradable coatings, such as by microencapsulation, multiple coatings, etc. Liposome delivery systems include, for example, small unilamellar vesicles, large unilamellar vesicles and multilamellar vesicles. In some embodiments, liposomes are formed from a variety of phospholipids, such as cholesterol, stearylamine or phosphatidylcholines. For oral administration in a capsule form, useful carriers, solvents or diluents include lactose, medium chain triglycerides, ethanol and dried corn starch.

In some embodiments, liquid preparations for oral administration take the form of, for example, solutions, syrups or suspensions, or they are suitably presented as a dry product for constitution with water or other suitable vehicle before use. When aqueous suspensions and/or emulsions are administered orally, the compound of the application is suitably suspended or dissolved in an oily phase that is combined with emulsifying and/or suspending agents. If desired, certain sweetening and/or flavoring and/or coloring agents are added. Such liquid preparations for oral administration are prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., medium chain triglycerides, almond oil, oily esters or ethyl alcohol); and preservatives (e.g., methyl or propyl p-hydroxybenzoates or sorbic acid). Useful diluents include lactose and high molecular weight polyethylene glycols.

It is also possible to freeze-dry the compounds of the application and use the lyophilizates obtained, for example, for the preparation of products for injection.

In some embodiments, a compound of the application is administered parenterally. For example, solutions of a compound of the application are prepared in water suitably mixed with a surfactant such as hydroxypropylcellulose. In some embodiments, dispersions are prepared in glycerol, liquid polyethylene glycols, DMSO and mixtures thereof with or without alcohol and in oils. Under ordinary conditions of storage and use, these preparations contain a preservative to prevent the growth of microorganisms. A person skilled in the art would know how to prepare suitable formulations. For parenteral administration, sterile solutions of the compounds of the application are usually prepared and the pH's of the solutions are suitably adjusted and buffered. For intravenous use, the total concentration of solutes should be controlled to render the preparation isotonic. For ocular administration, ointments or droppable liquids are delivered, for example, by ocular delivery systems known to the art such as applicators or eye droppers. In some embodiments, such compositions include mucomimetics such as hyaluronic acid, chondroitin sulfate, hydroxypropyl methylcellulose or polyvinyl alcohol, preservatives such as sorbic acid, EDTA or benzyl chromium chloride and the usual quantities of diluents or carriers. For pulmonary administration, diluents or carriers will be selected to be appropriate to allow the formation of an aerosol.

In some embodiments, a compound of the application is formulated for parenteral administration by injection, including using conventional catheterization techniques or infusion. Formulations for injection are, for example, presented in unit dosage form, e.g., in ampoules or in multi-dose containers, with an added preservative. In some embodiments, the compositions take such forms as sterile suspensions, solutions or emulsions in oily or aqueous vehicles and contain formulating agents such as suspending, stabilizing and/or dispersing agents. In all cases, the form must be sterile and must be fluid to the extent that easy syringability exists. Alternatively, the compounds of the application are suitably in a sterile powder form for reconstitution with a suitable vehicle, e.g., sterile pyrogen-free water, before use.

In some embodiments, compositions for nasal administration are conveniently formulated as aerosols, drops, gels and powders. For intranasal administration or administration by inhalation, the compounds of the application are conveniently delivered in the form of a solution, dry powder formulation or suspension from a pump spray container that is squeezed or pumped by the patient or as an aerosol spray presentation from a pressurized container or a nebulizer. Aerosol formulations typically comprise a solution or fine suspension of the active substance in a physiologically acceptable aqueous or non-aqueous solvent and are usually presented in single or multidose quantities in sterile form in a sealed container, which, for example, take the form of a cartridge or refill for use with an atomising device. Alternatively, the sealed container is a unitary dispensing device such as a single dose nasal inhaler or an aerosol dispenser fitted with a metering valve which is intended for disposal after use. Where the dosage form comprises an aerosol dispenser, it will contain a propellant which is, for example, a compressed gas such as compressed air or an organic propellant such as fluorochlorohydrocarbon. Suitable propellants include but are not limited to dichlorodifluoromethane, trichlorofluoromethane, dichlorotetrafluoroethane, heptafluoroalkanes, carbon dioxide or another suitable gas. In the case of a pressurized aerosol, the dosage unit is suitably determined by providing a valve to deliver a metered amount. In some embodiments, the pressurized container or nebulizer contains a solution or suspension of the active compound. Capsules and cartridges (made, for example, from gelatin) for use in an inhaler or insufflator are, for example, formulated containing a powder mix of a compound of the application and a suitable powder base such as lactose or starch. The aerosol dosage forms can also take the form of a pump-atomizer.

Compositions suitable for buccal or sublingual administration include tablets, lozenges and pastilles, wherein a compound of the application is formulated with a carrier such as sugar, acacia, tragacanth, or gelatin and glycerine. Compositions for rectal administration are conveniently in the form of suppositories containing a conventional suppository base such as cocoa butter.

Suppository forms of the compounds of the application are useful for vaginal, urethral and rectal administrations. Such suppositories will generally be constructed of a mixture of substances that is solid at room temperature but melts at body temperature. The substances commonly used to create such vehicles include but are not limited to theobroma oil (also known as cocoa butter), glycerinated gelatin, other glycerides, hydrogenated vegetable oils, mixtures of polyethylene glycols of various molecular weights and fatty acid esters of polyethylene glycol. See, for example: Remington's Pharmaceutical Sciences, 16th Ed., Mack Publishing, Easton, PA, 1980, pp. 1530-1533 for further discussion of suppository dosage forms.

In some embodiments a compound of the application is coupled with soluble polymers as targetable drug carriers. Such polymers include, for example, polyvinylpyrrolidone, pyran copolymer, polyhydroxypropylmethacrylamide-phenol, polyhydroxy-ethylaspartamide-phenol, or polyethyleneoxide-polylysine substituted with palmitoyl residues. Furthermore, in some embodiments, a compound of the application is coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and crosslinked or amphipathic block copolymers of hydrogels.

The compounds of the application are particularly amenable to administration with the air of nano-carrier systems, such as liposomes, micelles, nanoparticles, nano-emulsions, lipidic nano-systems and the like (see for example, Bhat, M. et al. Chem. and Phys. of Lipids, 2021, 236, 105053). Accordingly the present application includes a composition comprising one or more compounds of the application and one or more components of a nano-carrier system.

A compound of the application including pharmaceutically acceptable salts and/or solvates thereof is suitably used on their own but will generally be administered in the form of a pharmaceutical composition in which the one or more compounds of the application (the active ingredient) is in association with a pharmaceutically acceptable carrier. Depending on the mode of administration, the pharmaceutical composition will comprise from about 0.05 wt % to about 99 wt % or about 0.10 wt % to about 70 wt %, of the active ingredient and from about 1 wt % to about 99.95 wt % or about 30 wt % to about 99.90 wt % of a pharmaceutically acceptable carrier, all percentages by weight being based on the total composition.

In some embodiments, the compounds of the application including pharmaceutically acceptable salts and/or solvates thereof are used are administered in a composition comprising an additional therapeutic agent. Therefore the present application also includes a pharmaceutical composition comprising one of more compounds of the application, or pharmaceutically acceptable salts and/or solvates thereof and an additional therapeutic agent, and optionally one or more pharmaceutically acceptable excipients. In some embodiments, the additional therapeutic agent is another known agent useful for treatment of a disease, disorder or condition by activation of a serotonin receptor, for example those listed in the Methods and Uses section below. In some embodiments, the additional therapeutic agent is a psychoactive drug.

In the above, the term “a compound” also includes embodiments wherein one or more compounds are referenced.

### IV. Methods and Uses of the Compounds of the Application

The compounds of the application are serotonergic binding agents that act as agonists, partial agonists or positive allosteric modulators at a serotonin receptor, including 5-HT_{2A}.

Accordingly, the present application includes a method for activating a serotonin receptor in a cell, either in a biological sample or in a subject, comprising administering an effective amount of one or more compounds of the application to the cell,

The application also includes a use of the application for activating a serotonin receptor in a cell as well as a use of one or more compounds of the application for the preparation of a medicament for activating a serotonin receptor in a cell. The application further includes one or more compounds of the application for use in activating a serotonin receptor in a cell. In some embodiments, the method or use is for activating a serotonin receptor in and/or on a cell, either in a biological sample or in a patient.

In some embodiments, the activating a serotonin receptor is without a hallucinogenic effect in the subject or is with a reduced hallucinogenic effect in the subject compared to a hallucinogenic effect from administration of an equivalent dose of a hallucinogenic drug such as for example psilocybin, DMT or 5-MeO-DMT. In some embodiments, the activating a serotonin receptor is without a hallucinogenic effect in the subject or is with a reduced hallucinogenic effect in the subject compared to a hallucinogenic effect from administration of an equivalent dose of psilocybin.

Compounds of the application are useful for treating diseases, disorders or conditions by activating a serotonin receptor. Therefore, the compounds of the present application are useful as medicaments. Accordingly, the application also includes a compound of the application for use as a medicament.

The present application also includes a method of treating a disease, disorder or condition that is treatable by activation of a serotonin receptor comprising administering a therapeutically effective amount of one or more compounds of the application to a subject in need thereof. The present application also includes a method of treating a disease, disorder or condition by activation of a serotonin receptor comprising administering a therapeutically effective amount of one or more compounds of the application to a subject in need thereof.

The present application also includes a use of one or more compounds of the application for treatment of a disease, disorder or condition that is treatable by activation of a serotonin receptor as well as a use of one or more compounds of the application for the preparation of a medicament for treatment of a disease, disorder or condition that is treatable by activation of a serotonin receptor. The application further includes one or more compounds of the application for use in treating a disease, disorder or condition that is treatable by activation of a serotonin receptor.

The present application also includes a use of one or more compounds of the application for treatment of a disease, disorder or condition by activation of a serotonin receptor as well as a use of one or more compounds of the application for the preparation of a medicament for treatment of a disease, disorder or condition by activation of a serotonin receptor. The application further includes one or more compounds of the application for use in treating a disease, disorder or condition by activation of a serotonin receptor.

In some embodiments, the serotonin receptor is 5-HT_{2A}. Accordingly, the present application includes a method for activating 5-HT_{2A} in a cell, either in a biological sample or in a patient, comprising administering an effective amount of one or more compounds of the application to the cell. The application also includes a use of one or more compounds of the application for activating 5-HT_{2A} in a cell as well as a use of one or more compounds of the application for the preparation of a medicament for activating 5-HT_{2A} in a cell. The application further includes one or more compounds of the application for use in activating 5-HT_{2A} in a cell. In some embodiments, the method or use is for activating 5-HT₂ in and/or on a cell, either in a biological sample or in a patient.

The present application also includes a method of treating a disease, disorder or condition that is treatable by activation of 5-HT_{2A} comprising administering a therapeutically effective amount of one or more compounds of the application to a subject in need thereof.

The present application also includes a method of treating a disease, disorder or condition that is treatable by activation of 5-HT_{2A} comprising administering a therapeutically effective amount of one or more compounds of the application to a subject in need thereof. The present application also includes a use of one or more compounds of the application for treatment of a disease, disorder or condition that is treatable by activation of 5-HT_{2A} as well as a use of one or more compounds of the application for the preparation of a medicament for treatment of a disease, disorder or condition that is treatable by activation of 5-HT_{2A}. The application further includes one or more compounds of the application for use in treating a disease, disorder or condition that is treatable by activation of 5-HT_{2A}.

The present application also includes a method of treating a disease, disorder or condition by activation of 5-HT_{2A} comprising administering a therapeutically effective amount of one or more compounds of the application to a subject in need thereof. The present application also includes a use of one or more compounds of the application for treatment of a disease, disorder or condition by activation of 5-HT_{2A} as well as a use of one or more compounds of the application for the preparation of a medicament for treatment of a disease, disorder or condition by activation of 5-HT_{2A}. The application further includes one or more compounds of the application for use in treating a disease, disorder or condition by activation of 5-HT_{2A}.

In some embodiments, the treating of the disease, disorder or condition that is treatable by activation of a serotonin receptor is without a hallucinogenic effect in the subject or is with a reduced hallucinogenic effect in the subject compared to a hallucinogenic effect from administration of an equivalent dose of a hallucinogenic drug such as for example psilocybin, DMT or 5-MeO-DMT. In some embodiments, the hallucinogenic drug is psilocybin. In some embodiments, the treating of the disease, disorder or condition that is treatable by activation of a serotonin receptor including 5-HT_{2A} is without a hallucinogenic effect in the subject or is with a reduced hallucinogenic effect in the subject compared to a hallucinogenic effect from administration of an equivalent dose of psilocybin.

In some embodiments, the compounds of the application are useful for preventing, treating and/or reducing the severity of a mental illness disorder and/or condition in a subject. Therefore, in some embodiments, the disease, disorder or condition that is treated by activation of a serotonin receptor is a mental illness. Accordingly, the present application also includes a method of treating a mental illness comprising administering a therapeutically effective amount of one or more compounds of the application to a subject in need thereof. The present application also includes a use of one or more compounds of the application for treatment a mental illness, as well as a use of one or more compounds of the application for the preparation of a medicament for treatment of a mental illness. The application further includes one or more compounds of the application for use in treating a mental illness.

In some embodiments, the mental illness is selected from anxiety disorders such as generalized anxiety disorder, panic disorder, social anxiety disorder and specific phobias; depression such as, hopelessness, loss of pleasure, fatigue and suicidal thoughts; mood disorders, such as depression, bipolar disorder, cancer-related depression, anxiety and cyclothymic disorder; psychotic disorders, such as hallucinations, delusions, schizophrenia; impulse control and addiction disorders, such as pyromania (starting fires), kleptomania (stealing) and compulsive gambling; alcohol addiction; drug addiction, such as opioid addiction; personality disorders, such as antisocial personality disorder, obsessive-compulsive personality disorder and paranoid personality disorder; obsessive-compulsive disorder (OCD), such as thoughts or fears that cause a subject to perform certain rituals or routines; post-traumatic stress disorder (PTSD); stress response syndromes (formerly called adjustment disorders); dissociative disorders, formerly called multiple personality disorder, or “split personality,” and depersonalization disorder; factitious disorders; sexual and gender disorders, such as sexual dysfunction, gender identity disorder and the paraphilia's; somatic symptom disorders, formerly known as a psychosomatic disorder or somatoform disorder; and combinations thereof. In some embodiments, the mental illness is post-partum depression.

In some embodiments, the treating of the disease, disorder or condition that is treatable by activation of a serotonin receptor is without a hallucinogenic effect in the subject or is with a reduced hallucinogenic effect in the subject compared to a hallucinogenic effect from administration of an equivalent dose of a hallucinogenic drug such as for example psilocybin, DMT or 5-MeO-DMT. In some embodiments, the hallucinogenic drug is psilocybin In some embodiments, the treating of mental illness is without a hallucinogenic effect in the subject or is with a reduced hallucinogenic effect in the subject compared to a hallucinogenic effect from administration of an equivalent dose of psilocybin.

In some embodiments, the disease, disorder or condition that is treated by activation of a serotonin receptor comprises cognitive impairment; ischemia including stroke; neurodegeneration; refractory substance use disorders; sleep disorders; pain, such as social pain, acute pain, cancer pain, chronic pain, breakthrough pain, bone pain, soft tissue pain, nerve pain, referred pain, phantom pain, neuropathic pain, cluster headaches and migraine; obesity and eating disorders; epilepsies and seizure disorders; neuronal cell death; excitotoxic cell death; or a combination thereof.

In some embodiments, the disease, disorder or condition that is treated by activation of a serotonin receptor is neurodegeneration. In some embodiments, the disease, disorder or condition that is treated by activation of a serotonin receptor is reduced brain-derived neurotrophic factor (BDNF), mammalian target of rapamycin (mTOR) activation and/or inflammation.

In some embodiments, the mental illness is selected from hallucinations and delusions and a combination thereof.

In some embodiments, the hallucinations are selected from visual hallucinations, auditory hallucinations, olfactory hallucinations, gustatory hallucinations, tactile hallucinations, proprioceptive hallucinations, equilibrioceptive hallucinations, nociceptive hallucinations, thermoceptive hallucinations and chronoceptive hallucinations, and a combination thereof.

In some embodiments, the disease, disorder or condition that is treated by activation of a serotonin receptor is psychosis or psychotic symptoms. Accordingly, the present application also includes a method of treating psychosis or psychotic symptoms comprising administering a therapeutically effective amount of one or more compounds of the application to a subject in need thereof. The present application also includes a use of one or more compounds of the application for treatment of psychosis or psychotic symptoms, as well as a use of one or more compounds of the application for the preparation of a medicament for treatment of psychosis or psychotic symptoms. The application further includes one or more compounds of the application for use in treating psychosis or psychotic symptoms.

In some embodiments, administering to said subject in need thereof a therapeutically effective amount of the compounds of the application does not result in a worsening of psychosis or psychotic symptoms such as, but not limited to, hallucinations and delusions. In some embodiments, administering to said subject in need thereof a therapeutically effective amount of the compounds of the application results in an improvement of psychosis or psychotic symptoms such as, but not limited to, hallucinations and delusions. In some embodiments, administering to said subject in need thereof a therapeutically effective amount of the compounds of the application results in an improvement of psychosis or psychotic symptoms.

In some embodiments, the compounds of the application are useful for treating a central nervous system (CNS) disorder in a subject in need of therapy, comprising administering a therapeutically effective amount of a compound of formula I, or a pharmaceutically acceptable salt thereof to the subject.

Therefore, in some embodiments, the disease, disorder or condition that is treated by activation of a serotonin receptor is a central nervous system (CNS) disease, disorder or condition and/or a neurological disease, disorder or condition. Accordingly, the present application also includes a method of treating a CNS disease, disorder or condition and/or a neurological disease, disorder or condition comprising administering a therapeutically effective amount of one or more compounds of the application to a subject in need thereof. The present application also includes a use of one or more compounds of the application for treatment a CNS disease, disorder or condition and/or a neurological disease, disorder or condition, as well as a use of one or more compounds of the application for the preparation of a medicament for treatment of a CNS disease, disorder or condition and/or a neurological disease, disorder or condition. The application further includes one or more compounds of the application for use in treating a CNS disease, disorder or condition and/or a neurological disease, disorder or condition.

In some embodiments the CNS disease, disorder or condition and/or neurological disease, disorder or condition is selected from neurological diseases including neurodevelopmental diseases and neurodegenerative diseases such as Alzheimer's disease; presenile dementia; senile dementia; vascular dementia; Lewy body dementia; cognitive impairment, Parkinson's disease and Parkinsonian related disorders such as Parkinson dementia, corticobasal degeneration, and supranuclear palsy; epilepsy; CNS trauma; CNS infections; CNS inflammation; stroke; multiple sclerosis; Huntington's disease; mitochondrial disorders; Fragile X syndrome; Angelman syndrome; hereditary ataxias; neuro-otological and eye movement disorders; neurodegenerative diseases of the retina amyotrophic lateral sclerosis; tardive dyskinesias; hyperkinetic disorders; attention deficit hyperactivity disorder and attention deficit disorders; restless leg syndrome; Tourette's syndrome; schizophrenia; autism spectrum disorders; tuberous sclerosis; Rett syndrome; cerebral palsy; disorders of the reward system including eating disorders such as anorexia nervosa (“AN”) and bulimia nervosa (“BN”); and binge eating disorder (“BED”), trichotillomania, dermotillomania, nail biting; migraine; fibromyalgia; and peripheral neuropathy of any etiology, and combinations thereof.

In some embodiments, the compounds of the application are useful for treating endophenotypes and/or symptom clusters across a variety of neuropsychiatric and CNS disorder in a subject in need of therapy, comprising administering a therapeutically effective amount of a compound of general formula (I-A), or a pharmaceutically acceptable salt thereof to the subject.

Therefore, in some embodiments, the disease, disorder or condition that is treated by activation of a serotonin receptor is an endophenotype or symptom cluster associated with the disease, disorder or condition. Accordingly, the present application also includes a method of treating an endophenotype and/or symptom cluster associated with a disease, disorder or condition that is treated by activation of a serotonin receptor comprising administering a therapeutically effective amount of one or more compounds of the application to a subject in need thereof. The present application also includes a use of one or more compounds of the application for treatment an endophenotype and/or symptom cluster associated with a disease, disorder or condition that is treated by activation of a serotonin receptor, as well as a use of one or more compounds of the application for the preparation of a medicament for treatment of an endophenotype and/or symptom cluster associated with a disease, disorder or condition that is treated by activation of a serotonin receptor. The application further includes one or more compounds of the application for use in treating an endophenotype and/or symptom cluster associated with a disease, disorder or condition that is treated by activation of a serotonin receptor.

In some embodiments, the endophenotype and/or symptom cluster is associated with a disease, disorder or condition selected from a neuropsychiatric disease, disorder or condition, and/or nervous system disease, disorder or condition. In some embodiments, the nervous system disease is a CNS disease, disorder or condition.

In some embodiments, the endophenotype and/or symptom cluster is associated neurodevelopmental diseases and neurodegenerative diseases such as apathy, anhedonia, attentional impairments, memory impairments, negative emotional bias, hypomania, executive impairment, impulsivity, decreased mood, decreased libido, sensory gating, impaired prepulse inhibition, aggression, suicidal ideation, obesity, increased arousal, decreased arousal, hypersexuality, decreased exploration, hyperactivity, hypoactivity, sleep disturbance, incontinence, impaired social perception, ruminating thoughts, and combinations thereof.

In some embodiments the endophenotype and/or symptom cluster is selected from apathy, anhedonia, attentional impairments, memory impairments, negative emotional bias, hypomania, executive impairment, impulsivity, decreased mood, decreased libido, sensory gating, impaired prepulse inhibition, aggression, suicidal ideation, obesity, increased arousal, decreased arousal, hypersexuality, decreased exploration, hyperactivity, hypoactivity, sleep disturbance, incontinence, impaired social perception and ruminating thoughts.

In some embodiments, the subject is a mammal. In another embodiment, the subject is human. In some embodiments, the subject is a non-human animal. In some embodiments, the subject is canine. In some embodiments, the subject is feline. Accordingly, the compounds, methods and uses of the present application are directed to both human and veterinary diseases, disorders and conditions.

In some embodiments, the “subject in need thereof” is a subject having the disease, disorder or condition to be treated.

In some embodiments, the compounds of the application are useful for treating behavioral problems in subjects that are felines or canines.

Therefore, in some embodiments, the disease, disorder or condition that is treated by activation of a serotonin receptor is behavioral problems in subjects that are felines or canines. Accordingly, the present application also includes a method of treating a behavioral problem comprising administering a therapeutically effective amount of one or more compounds of the application to a non-human subject in need thereof. The present application also includes a use of one or more compounds of the application for treatment a behavioral problem in a non-human subject, as well as a use of one or more compounds of the application for the preparation of a medicament for treatment of a behavioral problem in a non-human subject. The application further includes one or more compounds of the application for use in treating a behavioral problem in a non-human subject.

In some embodiments, the behavioral problems are selected from, but are not limited to, anxiety, fear, stress, sleep disturbances, cognitive dysfunction, aggression, excessive noise making, scratching, biting and a combination thereof.

In some embodiments, the non-human subject is canine. In some embodiments, the non-human subject is feline.

The present application also includes a method of treating a disease, disorder or condition by activation of a serotonin receptor comprising administering a therapeutically effective amount of one or more compounds of the application in combination with another known agent useful for treatment of a disease, disorder or condition by activation of a serotonin receptor to a subject in need thereof. The present application also includes a use of one or more compounds of the application in combination with another known agent useful for treatment of a disease, disorder or condition by activation of a serotonin receptor for treatment of a disease, disorder or condition by activation of a serotonin receptor, as well as a use of one or more compounds of the application in combination with another known agent useful for treatment of a disease, disorder or condition by activation of a serotonin receptor for the preparation of a medicament for treatment of a disease, disorder or condition by activation of a serotonin receptor. The application further includes one or more compounds of the application in combination with another known agent useful for treatment of a disease, disorder or condition by activation of a serotonin receptor for use in treating a disease, disorder or condition by activation of a serotonin receptor.

In some embodiments, the disease, disorder or condition that is treated by activation of a serotonin receptor is a mental illness. In some embodiments, the mental illness is selected from hallucinations and delusions and a combination thereof. In some embodiments, the disease, disorder or condition that is treated by activation of a serotonin receptor is a central nervous system (CNS) disorder. In some embodiments, the disease, disorder or condition that is treated by activation of a serotonin receptor is psychosis or psychotic symptoms. In some embodiments, the disease, disorder or condition that is treated by activation of a serotonin receptor is behavioral problems in a non-human subject.

In some embodiments, the disease, disorder or condition that is treated by activation of a serotonin receptor is a mental illness and the one or more compounds of the application are administered in combination with one or more additional treatments for a mental illness.

In some embodiments, the additional treatments for a mental illness is selected from antipsychotics, including typical antipsychotics and atypical antipsychotics; antidepressants including selective serotonin reuptake inhibitors (SSRIs) and selective norepinephrine reuptake inhibitors (SNRIs), tricyclic antidepressants and monoamine oxidase inhibitors (MAOIs) (e.g. bupropion); anti-anxiety medication including benzodiazepines such as alprazolam; mood stabilizers such as lithium and anticonvulsants such carbamazepine, divalproex (valproic acid), lamotrigine, gabapentin and topiramate. In some embodiments, the anticonvulsants are leviteracetam or brivaracetam.

In some embodiments, the disease, disorder or condition that is treated by activation of a serotonin receptor is selected from attention deficit hyperactivity disorder and attention deficit disorder and a combination thereof. In some embodiments, the disease, disorder or condition that is treated by activation of a serotonin receptor is attention deficit hyperactivity disorder and/or attention deficit disorder and a combination thereof and the one or more compounds of the application are administered in combination with one or more additional treatments for attention deficit hyperactivity disorder and/or attention deficit disorder and a combination thereof. In some embodiments, the additional treatments for attention deficit hyperactivity disorder and/or attention deficit disorder and a combination thereof are selected from methylphenidate, atomoxetine and amphetamine and a combination thereof.

In some embodiments, the disease, disorder or condition that is treated by activation of a serotonin receptor is dementia or Alzheimer's disease and the one or more compounds of the application are administered in combination with one or more additional treatments for dementia or Alzheimer's disease. In some embodiments, the additional treatments for dementia and Alzheimer's disease are selected acetylcholinesterase inhibitors, NMDA antagonists and muscarinic agonists and antagonists, and nicotinic agonists.

In some embodiments, the acetylcholinesterase inhibitors are selected from donepezil, galantamine, rivastigmine, and phenserine, and combinations thereof.

In some embodiments, the NMDA antagonists are selected from MK-801, ketamine, phencyclidine, and memantine, and combinations thereof.

In some embodiments, the nicotinic agonists is nicotine, nicotinic acid, nicotinic alpha7 agonists or alpha2 beta4 agonists or combinations thereof.

In some embodiments, the muscarinic agonists is a muscarinic M1 agonist or a muscarinic M4 agonist, or combinations thereof.

In some embodiments, the muscarinic antagonist is a muscarinic M2 antagonist.

In some embodiments, the disease, disorder or condition that is treated by activation of a serotonin receptor is psychosis or psychotic symptoms and the one or more compounds of the application are administered in combination with one or more additional treatments for psychosis or psychotic symptoms. In some embodiments, the additional treatments for psychosis or psychotic symptom are selected typical antipsychotics and atypical antipsychotics.

In some embodiments, the typical antipsychotics are selected from acepromazine, acetophenazine, benperidol, bromperidol, butaperazine, carfenazine, chlorproethazine, chlorpromazine, chlorprothixene, clopenthixol, cyamemazine, dixyrazine, droperidol, fluanisone, flupentixol, fluphenazine, fluspirilene, haloperidol, levomepromazine, lenperone, Ioxapine, mesoridazine, metitepine, molindone, moperone, oxypertine, oxyprotepine, penfluridol, perazine, periciazine, perphenazine, pimozide, pipamperone, piperacetazine, pipotiazine, prochlorperazine, promazine, prothipendyl, spiperone, sulforidazine, thiopropazate, thioproperazine, thioridazine, thiothixene, timiperone, trifluoperazine, trifluperidol, triflupromazine and zuclopenthixol and combinations thereof.

In some embodiments, the atypical antipsychotics are selected from amoxapine, amisulpride, aripiprazole, asenapine, blonanserin, brexpiprazole, cariprazine, carpipramine, clocapramine, clorotepine, clotiapine, clozapine, iloperidone, levosulpiride, lurasidone, melperone, mosapramine, nemonapride, olanzapine, paliperidone, perospirone, quetiapine, remoxipride, reserpine, risperidone, sertindole, sulpiride, sultopride, tiapride, veralipride, ziprasidone and zotepine, and combinations thereof.

In some embodiments, the disease, disorder or condition that is treated by activation of a serotonin receptor is a mental illness and the one or more compounds of the application are administered in combination with one or more additional treatments for a mental illness.

In some embodiments, the additional treatments for a mental illness is selected typical antipsychotics and atypical antipsychotics.

In some embodiments, the compound of Formula I is a zwitterion, and the methods and uses of the application include the administration of use of a compound of Formula I and pharmaceutically acceptable salts, solvates, zwitterions and/or prodrugs thereof.

In some embodiments, the “subject in need thereof” is a subject having the disease, disorder or condition to be treated.

In some embodiments, the “subject in need thereof” is a subject expressing an endophenotype and/or symptom cluster to be treated.

In some embodiments, effective amounts vary according to factors such as the disease state, age, sex and/or weight of the subject or species. In some embodiments, the amount of a given compound or compounds that will correspond to an effective amount will vary depending upon factors, such as the given drug(s) or compound(s), the pharmaceutical formulation, the route of administration, the type of condition, disease or disorder, the identity of the subject being treated and the like, but can nevertheless be routinely determined by one skilled in the art.

In some embodiment, the compounds of the application are administered one, two, three or four times a year. In some embodiments, the compounds of the application are administered at least once a week. However, in another embodiment, the compounds are administered to the subject from about one time per two weeks, three weeks or one month. In another embodiment, the compounds are administered about one time per week to about once daily. In another embodiment, the compounds are administered 1, 2, 3, 4, 5 or 6 times daily. The length of the treatment period depends on a variety of factors, such as the severity of the disease, disorder or condition, the age of the subject, the concentration and/or the activity of the compounds of the application and/or a combination thereof. It will also be appreciated that the effective dosage of the compound used for the treatment may increase or decrease over the course of a particular treatment regime. Changes in dosage may result and become apparent by standard diagnostic assays known in the art. In some instances, chronic administration is required. For example, the compounds are administered to the subject in an amount and for duration sufficient to treat the subject.

In some embodiments, the compounds of the application are administered at doses that are hallucinogenic or psychotomimetic and taken in conjunction with psychotherapy or therapy and may occur once, twice, three, or four times a year. However, in some embodiments, the compounds are administered to the subject once daily, once every two days, once every 3 days, once a week, once every two weeks, once a month, once every two months, or once every three months at doses that are not hallucinogenic or psychotomimetic.

A compound of the application is either used alone or in combination with other known agents useful for treating diseases, disorders or conditions by activation of a serotonin receptor, such as the compounds of the application. When used in combination with other known agents useful in treating diseases, disorders by activation of a serotonin receptor, it is an embodiment that a compound of the application is administered contemporaneously with those agents. As used herein, “contemporaneous administration” of two substances to a subject means providing each of the two substances so that they are both active in the individual at the same time. The exact details of the administration will depend on the pharmacokinetics of the two substances in the presence of each other and can include administering the two substances within a few hours of each other, or even administering one substance within 24 hours of administration of the other, if the pharmacokinetics are suitable. Design of suitable dosing regimens is routine for one skilled in the art. In particular embodiments, two substances will be administered substantially simultaneously, i.e., within minutes of each other, or in a single composition that contains both substances. It is a further embodiment of the present application that a combination of agents is administered to a subject in a non-contemporaneous fashion. In some embodiments, a compound of the present application is administered with another therapeutic agent simultaneously or sequentially in separate unit dosage forms or together in a single unit dosage form. Accordingly, the present application provides a single unit dosage form comprising one or more compounds of the application, an additional therapeutic agent and a pharmaceutically acceptable carrier.

The dosage of a compound of the application varies depending on many factors such as the pharmacodynamic properties of the compound, the mode of administration, the age, health and weight of the recipient, the nature and extent of the symptoms, the frequency of the treatment and the type of concurrent treatment, if any and the clearance rate of the compound in the subject to be treated. One of skill in the art can determine the appropriate dosage based on the above factors. In some embodiments, one or more compounds of the application are administered initially in a suitable dosage that is adjusted as required, depending on the clinical response. Dosages will generally be selected to maintain a serum level of the one or more compounds of the application from about 0.01 μg/cc to about 1000 μg/cc, or about 0.1 μg/cc to about 100 μg/cc. As a representative example, oral dosages of one or more compounds of the application will range between about 10 μg per day to about 1000 mg per day for an adult, suitably about 10 μg per day to about 1000 mg per day, more suitably about 10 μg per day to about 500 mg per day. For parenteral administration, a representative amount is from about 0.0001 mg/kg to about 10 mg/kg, about 0.001 mg/kg to about 100 mg/kg, about 0.1 mg/kg to about 1000 mg/kg or about 1 mg/kg to about 10000 mg/kg will be administered. For oral administration, a representative amount is from about 0.001 μg/kg to about 10 mg/kg, about 0.1 μg/kg to about 100 mg/kg, about 0.01 μg/kg to about 1000 mg/kg or about 1 μg/kg to about 10000 mg/kg. For administration in suppository form, a representative amount is from about 0.1 mg/kg to about 10 mg/kg or about 0.1 mg/kg to about 100 mg/kg.

In some embodiments, dosages will generally be selected to maintain a serum level of the one or more compounds of the application from about 0.01 μg/cc to about 1000 μg/cc, or about 0.1 μg/cc to about 100 μg/cc. As a representative example, oral dosages of one or more compounds of the application will range between about 10 μg per day to about 1000 mg per day for an adult, suitably about 10 μg per day to about 500 mg per day, more suitably about 10 μg per day to about 200 mg per day. For parenteral administration, a representative amount is from about 0.0001 mg/kg to about 10 mg/kg, about 0.0001 mg/kg to about 1 mg/kg, about 0.01 mg/kg to about 0.1 mg/kg or about 0.0001 mg/kg to about 0.01 mg/kg will be administered.

For oral administration, a representative amount is from about 0.001 μg/kg to about 10 mg/kg, about 0.1 μg/kg to about 10 mg/kg, about 0.01 μg/kg to about 1 mg/kg or about 0.1 μg/kg to about 1 mg/kg. For administration in suppository form, a representative amount is from about 0.1 mg/kg to about 10 mg/kg or about 0.1 mg/kg to about 1 mg/kg.

In some embodiments of the application, compositions are formulated for oral administration and the one or more compounds are suitably in the form of tablets containing 0.1, 0.25, 0.5, 0.75, 1.0, 5.0, 10.0, 20.0, 25.0, 30.0, 40.0, 50.0, 60.0, 70.0, 75.0, 80.0, 90.0, 100.0, 150, 200, 250, 300, 350, 400, 450, 500, 550, 600, 650, 700, 750, 800, 850, 900, 950 or 1000 mg of active ingredient (one or more compounds of the application) per tablet. In some embodiments of the application the one or more compounds of the application are administered in a single daily, weekly or monthly dose or the total daily dose is divided into two, three or four daily doses.

In some embodiments, the compounds of the application are used or administered in an effective amount which comprises administration of doses or dosage regimens that are devoid of clinically meaningful psychedelic/psychotomimetic actions. In some embodiments, the compounds of the application are used or administered in an effective amount which comprises administration of doses or dosage regimens that provide clinical effects similar to those exhibited by a human plasma psilocin Cmax of 4 ng/mL or less and/or human 5-HT_{2A} human CNS receptor occupancy of 40% or less or those exhibited by a human plasma psilocin Cmax of 1 ng/mL or less and/or human 5-HT_{2A} human CNS receptor occupancy of 30% or less. In some embodiments, the compounds of the application are used or administered in an effective amount which comprises administration of doses or dosage regimens that provide clinical effects similar to those exhibited by a human plasma psilocin Cmax of 4 ng/mL or greater and/or human 5-HT_{2A} human CNS receptor occupancy of 40% or greater or those exhibited by a human plasma psilocin Cmax of 10 ng/mL or greater and/or human 5-HT_{2A} human CNS receptor occupancy of 60% or greater.

In some embodiments, the compounds of the application are used or administered in an effective amount which comprises administration of doses or dosage regimens that provide clinical effects similar to those exhibited by a human plasma psilocin Tmax in excess of 60 minutes, in excess of 120 minutes or in excess of 180 minutes.

To be clear, in the above, the term “a compound” also includes embodiments wherein one or more compounds are referenced. Likewise, the term “compounds of the application” also includes embodiments wherein only one compound is referenced.

In some embodiments, the methods and uses as described above comprise administration or use of an effective amount compound of 2-(5-methoxyindolin-3-yl)-N,N-dimethylethan-1-amine (II-1):

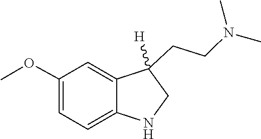

or a pharmaceutically acceptable salt, solvate and/or prodrug thereof, and/or 2-(indolin-3-yl)-N,N-dimethylethan-1-amine (11-2):

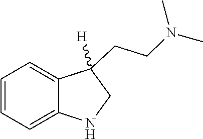

or a pharmaceutically acceptable salt, solvate and/or prodrug thereof, either on their own or in combination with one of more compounds of Formula I, and/or a salt, solvate and/or prodrug thereof, and/or one or more other therapeutic agents.

### V. Methods and Uses of Compounds of Formula II

The compounds of Formula II, or a pharmaceutically acceptable salt, solvate and/or prodrug, thereof are serotonergic binding agents that act as agonists, partial agonists or positive allosteric modulators at a serotonin receptor, including 5-HT_{2A}.

Accordingly, the present application includes a method for activating a serotonin receptor in and/or on a cell, either in a biological sample or in a subject, comprising administering an effective amount of one or more compounds of Formula II, or a pharmaceutically acceptable salt, solvate and/or prodrug thereof, to the cell,

The application also includes a use of one or more compounds of Formula II, or a pharmaceutically acceptable salt, solvate and/or prodrug thereof, for activating a serotonin receptor in and/or on a cell as well as a use of one or more compounds of Formula II, or a pharmaceutically acceptable salt, solvate, and/or prodrug thereof, for the preparation of a medicament for activating a serotonin receptor in and/or on a cell. The application further includes one or more compounds of Formula II, or a pharmaceutically acceptable salt, solvate, and/or prodrug thereof, for use in activating a serotonin receptor in and/or on a cell.

The compounds of Formula II, or a pharmaceutically acceptable salt, solvate and/or prodrug thereof, also show a significant reduction in their risk for causing hallucinogenic effects unlike other psychedelic 5-HT_{2A} agonists, such as psilocybin. Therefore, in some embodiments, the treating of the disease, disorder or condition that is treatable by activation of a serotonin receptor is without a hallucinogenic effect in the subject or is with a reduced hallucinogenic effect in the subject compared to a hallucinogenic effect from administration of an equivalent dose of a hallucinogenic drug such as for example psilocybin, DMT or 5-MeO-DMT. In some embodiments, the hallucinogenic drug is psilocybin. In some embodiments, the activating a serotonin receptor is without a hallucinogenic effect in the subject or is with a reduced hallucinogenic effect in the subject compared to a hallucinogenic effect from administration of an equivalent dose of psilocybin.

The application also includes a use of one or more compounds of Formula II, or a pharmaceutically acceptable salt, solvate, and/or prodrug thereof, for activating a serotonin receptor in and/or on a cell as well as a use of one or more compounds of Formula II, or a pharmaceutically acceptable salt, solvate and/or prodrug thereof, for the preparation of a medicament for activating a serotonin receptor in and/or on a cell. The application further includes one or more compounds of Formula II, or a pharmaceutically acceptable salt, solvate and/or prodrug thereof, for use in activating a serotonin receptor in and/or on a cell. In some embodiments, the treating of the disease, disorder or condition that is treatable by activation of a serotonin receptor is without a hallucinogenic effect in the subject or is with a reduced hallucinogenic effect in the subject compared to a hallucinogenic effect from administration of an equivalent dose of a hallucinogenic drug such as for example psilocybin, DMT or 5-MeO-DMT. In some embodiments, the hallucinogenic drug is psilocybin. In some embodiments, the activating a serotonin receptor is without a hallucinogenic effect in the subject or is with a reduced hallucinogenic effect in the subject compared to a hallucinogenic effect from administration of an equivalent dose of psilocybin.

The present application also includes a method of treating a disease, disorder or condition that is treatable by activation of a serotonin receptor comprising administering a therapeutically effective amount of one or more compounds of Formula II, or a pharmaceutically acceptable salt, solvate, and/or prodrug thereof, to a subject in need thereof, wherein treating of the disease, disorder or condition that is treatable by activation of a serotonin receptor is without a hallucinogenic effect in the subject or is with a reduced hallucinogenic effect in the subject compared to a hallucinogenic effect from administration of an equivalent dose a hallucinogenic drug. In some embodiments, the hallucinogenic drug is selected from psilocybin, DMT or 5-MeO-DMT. Therefore, the present application also includes a method of treating a disease, disorder or condition that is treatable by activation of a serotonin receptor comprising administering a therapeutically effective amount of one or more compounds of Formula II, or a pharmaceutically acceptable salt, solvate and/or prodrug thereof, to a subject in need thereof, wherein treating of the disease, disorder or condition that is treatable by activation of a serotonin receptor is without a hallucinogenic effect in the subject or is with a reduced hallucinogenic effect in the subject compared to a hallucinogenic effect from administration of an equivalent dose of psilocybin.

The present application also includes a use of one or more compounds of Formula II, or a pharmaceutically acceptable salt, solvate, and/or prodrug thereof, for treatment of a disease, disorder or condition by activation of a serotonin receptor wherein the use is without a hallucinogenic effect or is with a reduced hallucinogenic effect compared to an equivalent dose of a hallucinogenic drug, as well as a use of one or more compounds of Formula II, or a pharmaceutically acceptable salt, solvate, and/or prodrug thereof, for the preparation of a medicament for treatment of a disease, disorder or condition by activation of a serotonin receptor, wherein the use is without a hallucinogenic effect or is with a reduced hallucinogenic effect compared to an equivalent dose of a hallucinogenic drug. The application further includes one or more compounds of Formula II, or a pharmaceutically acceptable salt, solvate, and/or prodrug thereof, for use in treating a disease, disorder or condition by activation of a serotonin receptor, wherein the use is without a hallucinogenic effect or is with a reduced hallucinogenic effect compared to an equivalent dose of a hallucinogenic drug. In some embodiments, the hallucinogenic drug is selected from psilocybin, DMT or 5-MeO-DMT. Therefore, the present application also includes a use of one or more compounds of Formula II, or a pharmaceutically acceptable salt, solvate and/or prodrug thereof, for treatment of a disease, disorder or condition by activation of a serotonin receptor wherein the use is without a hallucinogenic effect or is with a reduced hallucinogenic effect compared to a hallucinogenic effect from use of an equivalent dose of psilocybin, as well as a use of one or more compounds of Formula II, or a pharmaceutically acceptable salt, solvate and/or prodrug thereof, for the preparation of a medicament for treatment of a disease, disorder or condition by activation of a serotonin receptor, wherein the use is without a hallucinogenic effect or is with a reduced hallucinogenic effect compared to a hallucinogenic effect from use of an equivalent dose of psilocybin. The application further includes one or more compounds of Formula II, or a pharmaceutically acceptable salt, solvate and/or prodrug thereof, for use in treating a disease, disorder or condition by activation of a serotonin receptor, wherein the use is without a hallucinogenic effect or is with a reduced hallucinogenic effect compared to a hallucinogenic effect from use of an equivalent dose of psilocybin.

In some embodiments, the serotonin receptor is 5-HT_{2A}.

The present application also includes a method of treating a disease, disorder or condition by activation of a serotonin receptor comprising administering a therapeutically effective amount of one or more compounds of Formula II, or a pharmaceutically acceptable salt, solvate and/or prodrug thereof, in combination with another known agent useful for treatment of a disease, disorder or condition by activation of a serotonin receptor to a subject in need thereof, wherein the treating a disease, disorder or condition by activation of a serotonin receptor is without a hallucinogenic effect in the subject or is with a reduced hallucinogenic effect in the subject compared to a hallucinogenic effect from administration of an equivalent dose of a hallucinogenic drug. In some embodiments, the hallucinogenic drug is selected from psilocybin, DMT or 5-MeO-DMT. Therefore, the present application also includes a method of treating a disease, disorder or condition by activation of a serotonin receptor comprising administering a therapeutically effective amount of one or more compounds of Formula II, or a pharmaceutically acceptable salt, solvate and/or prodrug thereof, in combination with another known agent useful for treatment of a disease, disorder or condition by activation of a serotonin receptor to a subject in need thereof, wherein the treating a disease, disorder or condition by activation of a serotonin receptor is without a hallucinogenic effect in the subject or is with a reduced hallucinogenic effect in the subject compared to a hallucinogenic effect from administration of an equivalent dose of psilocybin.

The present application also includes a use of one or more compounds of Formula II, or a pharmaceutically acceptable salt, solvate and/or prodrug thereof, in combination with another known agent useful for treatment of a disease, disorder or condition by activation of a serotonin receptor for treatment of a disease, disorder or condition by activation of a serotonin receptor wherein the use is without a hallucinogenic effect or is with a reduced hallucinogenic effect compared to a hallucinogenic effect from use of an equivalent dose of a hallucinogenic drug, as well as a use of one or more compounds of Formula II, or a pharmaceutically acceptable salt, solvate and/or prodrug thereof, in combination with another known agent useful for treatment of a disease, disorder or condition by activation of a serotonin receptor for the preparation of a medicament for treatment of a disease, disorder or condition by activation of a serotonin receptor wherein the use is without a hallucinogenic effect or is with a reduced hallucinogenic effect compared to a hallucinogenic effect from use of an equivalent dose of a hallucinogenic drug. The application further includes one or more compounds of Formula II, or a pharmaceutically acceptable salt, solvate and/or prodrug thereof, in combination with another known agent useful for treatment of a disease, disorder or condition by activation of a serotonin receptor for use in treating a disease, disorder or condition by activation of a serotonin receptor, wherein the use is without a hallucinogenic effect or is with a reduced hallucinogenic effect compared to a hallucinogenic drug effect from use of an equivalent dose of a hallucinogenic drug. In some embodiments, the hallucinogenic drug is selected from psilocybin, DMT or 5-MeO-DMT. Therefore, The present application also includes a use of one or more compounds of Formula II, or a pharmaceutically acceptable salt, solvate and/or prodrug thereof, in combination with another known agent useful for treatment of a disease, disorder or condition by activation of a serotonin receptor for treatment of a disease, disorder or condition by activation of a serotonin receptor wherein the use is without a hallucinogenic effect or is with a reduced hallucinogenic effect compared to a hallucinogenic effect from use of an equivalent dose of psilocybin, as well as a use of one or more compounds of Formula II, or a pharmaceutically acceptable salt, solvate and/or prodrug thereof, in combination with another known agent useful for treatment of a disease, disorder or condition by activation of a serotonin receptor for the preparation of a medicament for treatment of a disease, disorder or condition by activation of a serotonin receptor wherein the use is without a hallucinogenic effect or is with a reduced hallucinogenic effect compared to a hallucinogenic effect from use of an equivalent dose of psilocybin. The application further includes one or more compounds of Formula II, or a pharmaceutically acceptable salt, solvate and/or prodrug thereof, in combination with another known agent useful for treatment of a disease, disorder or condition by activation of a serotonin receptor for use in treating a disease, disorder or condition by activation of a serotonin receptor, wherein the use is without a hallucinogenic effect or is with a reduced hallucinogenic effect compared to a hallucinogenic effect from use of an equivalent dose of psilocybin.

The embodiments for a disease, disorder or condition treatable by activation of a serotonin receptor are the same as the embodiments for a disease, disorder or condition treatable by activation of a serotonin receptor described above with respect to the one or more compounds of the invention, i.e. compounds of Formula I, or a pharmaceutically acceptable salt, solvate and/or prodrug thereof.

In some embodiments, the disease, disorder or condition that is treated by activation of a serotonin receptor is an endophenotype or symptom cluster associated with the disease, disorder or condition as described above with respect to the one or more compounds of the invention, i.e. compounds of Formula I, or a pharmaceutically acceptable salt, solvate and/or prodrug thereof.

The embodiments for a “known agent useful for treatment of a disease, disorder or condition by activation of a serotonin receptor” are the same as the embodiments for a “known agent useful for treatment of a disease, disorder or condition by activation of a serotonin receptor” described above with respect to the one or more compounds of the invention, i.e. compounds of Formula I, or a pharmaceutically acceptable salt, solvate and/or prodrug thereof.

In some embodiments, the “subject in need thereof” is a subject having the disease, disorder or condition to be treated.

The embodiments for “subject” are the same as the embodiments for “subject” described above for administration or use with the one or more compounds of the invention.

A compound of Formula II, or a pharmaceutically acceptable salt, solvate and/or prodrug thereof, is:

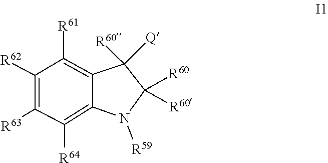

wherein:
R⁵⁹ is selected from C(O)R⁶⁵, CO₂R⁶⁵, C(O)N(R⁶⁵)(R⁶⁵), S(O)R⁶⁵, SO₂R⁶⁵, C₁₋₆alkyleneR⁶⁵ and R⁶⁵;
Q′ is Q3′:

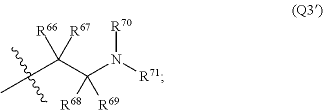

[symbol] over a bond means that the bond is attached to a remaining portion of the compound;
R⁶⁰, R^{60′} and R^{60″} are independently selected from H, halo, C₁₋₆alkyl, C₁₋₆alkoxy, C₁₋₆ alkyleneN(R^{60a})(R^{60b}) and SC₁₋₆alkyl;
R^{60a} and R^{60b} are independently selected from H and C₁₋₆alkyl;
R⁶¹, R⁶², R⁶³ and R⁶⁴ are independently selected from H, halo, N(R^{63′})(R^{64′}), C₁₋₆alkyl, C₁₋₆ alkoxy, C₁₋₆alkyleneN(R^{63′})(R^{64′}) and SC₁₋₆alkyl, the latter four groups being optionally substituted with one or two substituents selected from OH and C₁₋₆alkoxy, or
two adjacent of R⁶¹, R⁶², R⁶³ and R⁶⁴ are linked together to form O—(CH₂)₁₋₂O and the remaining of R⁶¹, R⁶², R⁶³ and R⁶⁴ are independently selected from H, halo, C₁₋₆alkyl and C₁₋₆alkoxy, N(R^{63′})(R⁶⁴), C₁₋₆alkyleneN(R^{63′})(R^{64′}) and SC₁₋₆alkyl, or
one of R⁶¹, R⁶², R⁶³ and R⁶⁴ is selected from A′, O-A′ and C₁₋₄alkyleneA′ and the remaining of R⁶¹, R⁶², R⁶³ and R⁶⁴ are independently H or halo;
R^{63′} and R^{64′} are independently selected from H and C₁₋₆alkyl;
A′ is selected from phenyl, C₃₋₆cycloalkyl, 3- to 6-membered heterocycloalkyl comprising 1 to 4 heteromoeities independently selected from O, S, S(O), SO₂, N and NR⁷² and 5- to 6-membered heteroaryl comprising 1 to 4 heteromoeities independently selected from O, S, S(O), SO₂, N and NR⁷², wherein the phenyl, C₃₋₁₀cycloalkyl, 3- to 6-membered heterocycloalkyl and 5- to 6-membered heteroaryl are optionally substituted with one or more substituents independently selected from halo, C₁₋₄alkyl, OC₁₋₄alkyl, N(R^{63′})(R^{64′}), C₁₋₆alkyleneN(R^{63′})(R^{64′}) and SC₁₋₆alkyl;
R⁶⁵ is selected from H, C₁₋₆alkyl, C₂₋₆alkenyl, C₂₋₆alkynyl, phenyl, C₃₋₆cycloalkyl, 3- to 6-membered heterocycloalkyl comprising 1 to 4 heteromoeities independently selected from O, S, N and NR^{65″} and 5- to 6-membered heteroaryl comprising 1 to 4 heteromoeities independently selected from O, S, N and NR^{65″}, wherein the latter 5 groups are optionally substituted with one or more substituents independently selected from halo, OR⁷³, N(R⁷³)(R⁷⁴) and SR⁷³ and/or the C₁₋₆alkyl is optionally interrupted by one to three heteromoieties independently selected from O, C(O), CO₂ and NR⁷⁵;
R^{65′} is selected from H and C₁₋₆alkyl;
R^{65″} is selected from H and C₁₋₆alkyl;
R⁶⁶, R⁶⁷, R⁶⁸ and R⁶⁹ are independently selected from H, halo and C₁₋₆alkyl;
R⁷⁰ and R⁷¹ are independently selected from H, C₁₋₆alkyl and C(O)C₁₋₆alkyl, or
R⁷⁰ and R⁷¹, together with the N atom to which they are bound, form a 3- to 6-membered heterocyclic ring which optionally comprises one or two additional heteromoieties independently selected from O, S, S(O), SO₂, N, and NR⁷⁶;
R⁷², R⁷³, R⁷⁴, R⁷⁵ and R⁷⁶ are independently selected from H and C₁₋₆alkyl;
R^{72a} and R^{72b} are independently selected from H and C₁₋₆alkyl; and
all available hydrogen atoms are optionally and independently substituted with a fluorine atom or chlorine atom and all available atoms are optionally substituted with alternate isotope thereof.

The embodiments for R⁵⁹, R⁶⁰, R^{60′}, R^{60″}, R^{60a}, R^{60b}, R⁶¹, R⁶², R⁶³, R⁶⁴, R^{63′} and R^{64′} R⁶⁵, R^{65′}, R^{65″}, R⁶⁶, R⁶⁷, R⁶⁸, R⁶⁹, R⁷⁰, R⁷¹, R⁷², R^{72a}, R^{72b} R⁷³, R⁷⁴, R⁷⁵, R⁷⁶, Q′ and A′ in the compounds of Formula II are the same as the embodiments for R¹, R², R^{2′}, R²″, R^{2a}, R^{2b}, R³, R⁴, R⁵, R⁶, R^{5′}, R^{6′}, R⁷, R^{7′}, R⁷″, R²⁶, R²⁷, R²⁸, R²⁹, R³⁰, R³¹, R⁵⁴, R^{54a}, R^{54b} R⁵⁵, R⁵⁶, R⁵⁷, R⁵⁸, Q and A respectively for Formula I as described above with the exception that the provisos in Formula I do not apply to Formula II. Further, in some embodiments of Formula II, the compound of Formula II is selected from 2-(5-methoxyindolin-3-yl)-N,N-dimethylethan-1-amine (II-1) and 2-(Indolin-3-yl)-N,N-dimethylethan-1-amine (II-2), and pharmaceutically acceptable salts, solvates and/or prodrugs thereof.

In some embodiments, the compound of Formula II or a pharmaceutically acceptable salt, solvate and/or prodrug thereof is a compound of Formula I or a pharmaceutically acceptable salt, solvate and/or prodrug thereof. In some embodiments, the compound of Formula II is selected from the compounds of Formula I listed in Table A, or a pharmaceutically acceptable salt, solvate and/or prodrug thereof.

Further, in some embodiments, the compound of Formula II is selected from the compounds listed below, or a pharmaceutically acceptable salt, solvate and/or prodrug thereof:

| Compound I.D. | Chemical Name | Structure |
|---|---|---|
| II-1 | 2-(5-Methoxyindolin-3-yl)- N,N-dimethylethan-1-amine | 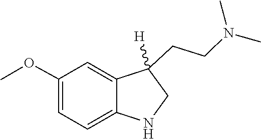 and |
| II-2 | 2-(Indolin-3-yl)-N,N- dimethylethan-1-amine (II-2). | 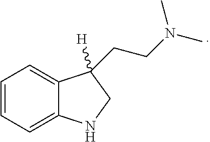 |

In some embodiments, the compound of Formula II is a zwitterion, and the present application includes a compound of Formula II and pharmaceutically acceptable salts, solvates, zwitterions and/or prodrugs thereof.

In some embodiments, the pharmaceutically acceptable salt is an acid addition salt or a base addition salt. The selection of a suitable salt may be made by a person skilled in the art. Suitable salts include acid addition salts that may, for example, be formed by mixing a solution of a compound with a solution of a pharmaceutically acceptable acid such as hydrochloric acid, sulfuric acid, acetic acid, trifluoroacetic acid, or benzoic acid. Additionally, acids that are generally considered suitable for the formation of pharmaceutically useful salts from basic pharmaceutical compounds are discussed, for example, by P. Stahl et al, Camille G. (eds.) and Handbook of Pharmaceutical Salts. Properties, Selection and Use. (2002) Zurich: Wiley VCH; S. Berge et al, Journal of Pharmaceutical Sciences 1977 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website).

An acid addition salt suitable for, or compatible with, the treatment of subjects is any non-toxic organic or inorganic acid addition salt of any basic compound. Basic compounds that form an acid addition salt include, for example, compounds comprising an amine group. Illustrative inorganic acids which form suitable salts include hydrochloric, hydrobromic, sulfuric, nitric and phosphoric acids, as well as acidic metal salts such as sodium monohydrogen orthophosphate and potassium hydrogen sulfate. Illustrative organic acids which form suitable salts include mono-, di- and tricarboxylic acids. Illustrative of such organic acids are, for example, acetic, trifluoroacetic, propionic, glycolic, lactic, pyruvic, malonic, succinic, glutaric, fumaric, malic, tartaric, citric, ascorbic, maleic, hydroxymaleic, benzoic, hydroxybenzoic, phenylacetic, cinnamic, mandelic, salicylic, 2-phenoxybenzoic, p-toluenesulfonic acid and other sulfonic acids such as methanesulfonic acid, ethanesulfonic acid and 2-hydroxyethanesulfonic acid. In some embodiments, exemplary acid addition salts also include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates (“mesylates”), naphthalenesulfonates, nitrates, oxalates, phosphates, propionates, salicylates, succinates, sulfates, tartarates, thiocyanates, toluenesulfonates (also known as tosylates) and the like. In some embodiments, the mono- or di-acid salts are formed and such salts exist in either a hydrated, solvated or substantially anhydrous form. In general, acid addition salts are more soluble in water and various hydrophilic organic solvents and generally demonstrate higher melting points in comparison to their free base forms. The selection criteria for the appropriate salt will be known to one skilled in the art. Other non-pharmaceutically acceptable salts such as but not limited to oxalates may be used, for example in the isolation of compounds of Formula II for laboratory use, or for subsequent conversion to a pharmaceutically acceptable acid addition salt.

A base addition salt suitable for, or compatible with, the treatment of subjects is any non-toxic organic or inorganic base addition salt of any acidic compound. Acidic compounds that form a basic addition salt include, for example, compounds comprising a carboxylic acid group. Illustrative inorganic bases which form suitable salts include lithium, sodium, potassium, calcium, magnesium or barium hydroxide as well as ammonia. Illustrative organic bases which form suitable salts include aliphatic, alicyclic or aromatic organic amines such as isopropylamine, methylamine, trimethylamine, picoline, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like. Exemplary organic bases are isopropylamine, diethylamine, ethanolamine, trimethylamine, dicyclohexylamine, choline and caffeine. The selection of the appropriate salt may be useful, for example, so that an ester functionality, if any, elsewhere in a compound is not hydrolyzed. The selection criteria for the appropriate salt will be known to one skilled in the art. In some embodiments, exemplary basic salts also include ammonium salts, alkali metal salts such as sodium, lithium and potassium salts, alkaline earth metal salts such as calcium and magnesium salts, salts with organic bases (for example, organic amines) such as dicyclohexylamine, Abutyl amine, choline and salts with amino acids such as arginine, lysine and the like. Basic nitrogen containing groups may be quarternized with agents such as lower alkyl halides (e.g., methyl, ethyl and butyl chlorides, bromides and iodides), dialkyl sulfates (e.g., dimethyl, diethyl and dibutyl sulfates), long chain halides (e.g., decyl, lauryl and stearyl chlorides, bromides and iodides), aralkyl halides (e.g., benzyl and phenethyl bromides) and others. Compounds carrying an acidic moiety can be mixed with suitable pharmaceutically acceptable salts to provide, for example, alkali metal salts (e.g., sodium or potassium salts), alkaline earth metal salts (e.g., calcium or magnesium salts) and salts formed with suitable organic ligands such as quaternary ammonium salts. Also, in the case of an acid (—COOH) or alcohol group being present, pharmaceutically acceptable esters can be employed to modify the solubility or hydrolysis characteristics of the compound.

All such acid salts and base salts are intended to be pharmaceutically acceptable salts of the compounds of Formula II within the scope of the application and all acid and base salts are considered equivalent to the free forms of the corresponding compounds for purposes of the application. In addition, when a compound of Formula II contains both a basic moiety, such as, but not limited to an aliphatic primary, secondary, tertiary or cyclic amine, an aromatic or heteroaryl amine, pyridine or imidazole and an acidic moiety, such as, but not limited to tetrazole or carboxylic acid, zwitterions (“inner salts”) may be formed and are included within the scope of the present application. It is understood that certain compounds of Formula II may exist in zwitterionic form, having both anionic and cationic centers within the same compound and a net neutral charge. Such zwitterions are included within the application.

Solvates of compounds of Formula II, or salts and/or prodrugs thereof, include, for example, those made with solvents that are pharmaceutically acceptable. Examples of such solvents include water (resulting solvate is called a hydrate) and ethanol and the like. Suitable solvents are physiologically tolerable at the dosage administered.

Prodrugs of compounds of Formula II, or salts and/or solvates thereof, include, for example, conventional esters formed with available hydroxy, thiol, amino or carboxyl groups. Some common esters which have been utilized as prodrugs are phenyl esters, aliphatic (C₁-C₂₄) esters, acyloxymethyl esters, carbamates and amino acid esters.

It is understood and appreciated that in some embodiments, compounds of Formula II, or a pharmaceutically acceptable salt, solvate and/or prodrug thereof, may have at least one chiral center and therefore can exist as enantiomers and/or diastereomers. It is to be understood that all such isomers and mixtures thereof in any proportion are encompassed within the scope of the present application. It is to be further understood that while the stereochemistry of the compounds may be as shown in any given compound listed herein, such compounds may also contain certain amounts (for example, less than 20%, suitably less than 10%, more suitably less than 5%) of compounds of Formula II having an alternate stereochemistry. It is intended that any optical isomers, as separated, pure or partially purified optical isomers or racemic mixtures thereof are included within the scope of the present application.

In some embodiments, the compounds of Formula II can also include tautomeric forms, such as keto-enol tautomers and the like. Tautomeric forms can be in equilibrium or sterically locked into one form by appropriate substitution. It is intended that any tautomeric forms which the compounds form, as well as mixtures thereof, are included within the scope of the present application.

The compounds of Formula II, and salts and/or solvates thereof, may further exist in varying amorphous and polymorphic forms and it is contemplated that any amorphous forms, polymorphs, or mixtures thereof, which form are included within the scope of the present application.

The compounds of Formula II, or salts, solvates and/or prodrugs thereof, may further be radiolabeled and accordingly all radiolabeled versions of the compounds of the application are included within the scope of the present application. Therefore, the compounds of Formula II also include those in which one or more radioactive atoms are incorporated within their structure.

The compounds of Formula II, or a pharmaceutically acceptable salt, solvate and/or prodrug thereof, are suitably formulated in a conventional manner into compositions using one or more carriers. The compounds of Formula II, or a pharmaceutically acceptable salt, solvate and/or prodrug thereof, are suitably formulated into pharmaceutical compositions for administration to subjects in a biologically compatible form suitable for administration in vivo. Accordingly, the present application further includes a pharmaceutical composition comprising one or more compounds of Formula II, or a pharmaceutically acceptable salt, solvate and/or prodrug thereof, and a pharmaceutically acceptable carrier wherein the one or more compounds of Formula II, or a pharmaceutically acceptable salt, solvate and/or prodrug thereof are present in an amount effective to treat of any of the diseases, disorders or conditions described herein.

The compounds of Formula II, or a pharmaceutically acceptable salt, solvate and/or prodrug thereof, are administered to a subject in a variety of forms depending on the selected route of administration, as will be understood by those skilled in the art. For example, compounds of Formula II are administered by oral, inhalation, parenteral, buccal, sublingual, insufflation, epidurally, nasal, rectal, vaginal, patch, pump, minipump, topical or transdermal administration and the pharmaceutical compositions formulated accordingly. In some embodiments, administration is by means of a pump for periodic or continuous delivery. Conventional procedures and ingredients for the selection and preparation of suitable compositions are described, for example, in Remington's Pharmaceutical Sciences (2000-20^{th} edition) and in The United States Pharmacopeia: The National Formulary (USP 24 NF19) published in 1999.

Parenteral administration includes systemic delivery routes other than the gastrointestinal (GI) tract and includes, for example intravenous, intra-arterial, intraperitoneal, subcutaneous, intramuscular, transepithelial, nasal, intrapulmonary (for example, by use of an aerosol), intrathecal, rectal and topical (including the use of a patch or other transdermal delivery device) modes of administration. Parenteral administration may be by continuous infusion over a selected period of time.

In some embodiments, a compound of Formula II, or a pharmaceutically acceptable salt, solvate and/or prodrug thereof, is orally administered, for example, with an inert diluent or with an assimilable edible carrier, or it is enclosed in hard or soft shell gelatin capsules, or it is compressed into tablets, or it is incorporated directly with the food of the diet. In some embodiments, the compound is incorporated with excipient and used in the form of ingestible tablets, buccal tablets, troches, capsules, caplets, pellets, granules, lozenges, chewing gum, powders, syrups, elixirs, wafers, aqueous solutions and suspensions and the like. In the case of tablets, carriers that are used include lactose, corn starch, sodium citrate and salts of phosphoric acid. Pharmaceutically acceptable excipients include binding agents (e.g., pregelatinized maize starch, polyvinylpyrrolidone or hydroxypropyl methylcellulose); fillers (e.g., lactose, microcrystalline cellulose or calcium phosphate); lubricants (e.g., magnesium stearate, talc or silica); disintegrants (e.g., potato starch or sodium starch glycolate); or wetting agents (e.g., sodium lauryl sulphate), or solvents (e.g. medium chain triglycerides, ethanol, water). In embodiments, the tablets are coated by methods well known in the art. In the case of tablets, capsules, caplets, pellets or granules for oral administration, pH sensitive enteric coatings, such as Eudragits™ designed to control the release of active ingredients are optionally used. Oral dosage forms also include modified release, for example immediate release and timed-release, formulations. Examples of modified-release formulations include, for example, sustained-release (SR), extended-release (ER, XR, or XL), time-release or timed-release, controlled-release (CR), or continuous-release (CR or Contin), employed, for example, in the form of a coated tablet, an osmotic delivery device, a coated capsule, a microencapsulated microsphere, an agglomerated particle, e.g., as of molecular sieving type particles, or, a fine hollow permeable fiber bundle, or chopped hollow permeable fibers, agglomerated or held in a fibrous packet. Timed-release compositions are formulated, for example as liposomes or those wherein the active compound is protected with differentially degradable coatings, such as by microencapsulation, multiple coatings, etc. Liposome delivery systems include, for example, small unilamellar vesicles, large unilamellar vesicles and multilamellar vesicles. In some embodiments, liposomes are formed from a variety of phospholipids, such as cholesterol, stearylamine or phosphatidylcholines. For oral administration in a capsule form, useful carriers, solvents or diluents include lactose, medium chain triglycerides, ethanol and dried corn starch.

In some embodiments, liquid preparations for oral administration take the form of, for example, solutions, syrups or suspensions, or they are suitably presented as a dry product for constitution with water or other suitable vehicle before use. When aqueous suspensions and/or emulsions are administered orally, the compound of Formula II, or a pharmaceutically acceptable salt, solvate and/or prodrug thereof, is suitably suspended or dissolved in an oily phase that is combined with emulsifying and/or suspending agents. If desired, certain sweetening and/or flavoring and/or coloring agents are added. Such liquid preparations for oral administration are prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., medium chain triglycerides, almond oil, oily esters or ethyl alcohol); and preservatives (e.g., methyl or propyl p-hydroxybenzoates or sorbic acid). Useful diluents include lactose and high molecular weight polyethylene glycols.

It is also possible to freeze-dry the compounds of Formula II, or a pharmaceutically acceptable salt, solvate and/or prodrug thereof, and use the lyophilizates obtained, for example, for the preparation of products for injection.

In some embodiments, a compound of Formula II, or a pharmaceutically acceptable salt, solvate and/or prodrug thereof, is administered parenterally. For example, solutions of a compound of Formula II, or a pharmaceutically acceptable salt, solvate and/or prodrug thereof, are prepared in water suitably mixed with a surfactant such as hydroxypropylcellulose. In some embodiments, dispersions are prepared in glycerol, liquid polyethylene glycols, DMSO and mixtures thereof with or without alcohol and in oils. Under ordinary conditions of storage and use, these preparations contain a preservative to prevent the growth of microorganisms. A person skilled in the art would know how to prepare suitable formulations. For parenteral administration, sterile solutions of the compounds of Formula II, or a pharmaceutically acceptable salt, solvate and/or prodrug thereof, are usually prepared and the pH's of the solutions are suitably adjusted and buffered. For intravenous use, the total concentration of solutes should be controlled to render the preparation isotonic. For ocular administration, ointments or droppable liquids are delivered, for example, by ocular delivery systems known to the art such as applicators or eye droppers. In some embodiments, such compositions include mucomimetics such as hyaluronic acid, chondroitin sulfate, hydroxypropyl methylcellulose or polyvinyl alcohol, preservatives such as sorbic acid, EDTA or benzyl chromium chloride and the usual quantities of diluents or carriers. For pulmonary administration, diluents or carriers will be selected to be appropriate to allow the formation of an aerosol.

In some embodiments, a compound of Formula II, or a pharmaceutically acceptable salt, solvate and/or prodrug thereof, is formulated for parenteral administration by injection, including using conventional catheterization techniques or infusion. Formulations for injection are, for example, presented in unit dosage form, e.g., in ampoules or in multi-dose containers, with an added preservative. In some embodiments, the compositions take such forms as sterile suspensions, solutions or emulsions in oily or aqueous vehicles and contain formulating agents such as suspending, stabilizing and/or dispersing agents. In all cases, the form should be sterile and must be fluid to the extent that easy syringability exists. Alternatively, the compounds of Formula II, or a pharmaceutically acceptable salt, solvate and/or prodrug thereof, are suitably in a sterile powder form for reconstitution with a suitable vehicle, e.g., sterile pyrogen-free water, before use.

In some embodiments, compositions for nasal administration are conveniently formulated as aerosols, drops, gels and powders. For intranasal administration or administration by inhalation, the compounds of Formula II, or a pharmaceutically acceptable salt, solvate and/or prodrug thereof, are conveniently delivered in the form of a solution, dry powder formulation or suspension from a pump spray container that is squeezed or pumped by the patient or as an aerosol spray presentation from a pressurized container or a nebulizer. Aerosol formulations typically comprise a solution or fine suspension of the active substance in a physiologically acceptable aqueous or non-aqueous solvent and are usually presented in single or multidose quantities in sterile form in a sealed container, which, for example, take the form of a cartridge or refill for use with an atomising device. Alternatively, the sealed container is a unitary dispensing device such as a single dose nasal inhaler or an aerosol dispenser fitted with a metering valve which is intended for disposal after use. Where the dosage form comprises an aerosol dispenser, it will contain a propellant which is, for example, a compressed gas such as compressed air or an organic propellant such as fluorochlorohydrocarbon. Suitable propellants include but are not limited to dichlorodifluoromethane, trichlorofluoromethane, dichlorotetrafluoroethane, heptafluoroalkanes, carbon dioxide or another suitable gas. In the case of a pressurized aerosol, the dosage unit is suitably determined by providing a valve to deliver a metered amount. In some embodiments, the pressurized container or nebulizer contains a solution or suspension of the active compound. Capsules and cartridges (made, for example, from gelatin) for use in an inhaler or insufflator are, for example, formulated containing a powder mix of a compound of Formula II, or a pharmaceutically acceptable salt, solvate and/or prodrug thereof, and a suitable powder base such as lactose or starch. The aerosol dosage forms can also take the form of a pump-atomizer.

Compositions suitable for buccal or sublingual administration include tablets, lozenges and pastilles, wherein a compound of Formula II, or a pharmaceutically acceptable salt, solvate and/or prodrug thereof, is formulated with a carrier such as sugar, acacia, tragacanth, or gelatin and glycerine. Compositions for rectal administration are conveniently in the form of suppositories containing a conventional suppository base such as cocoa butter.

Suppository forms of the compounds of Formula II, or a pharmaceutically acceptable salt, solvate and/or prodrug thereof, are useful for vaginal, urethral and rectal administrations. Such suppositories will generally be constructed of a mixture of substances that is solid at room temperature but melts at body temperature. The substances commonly used to create such vehicles include but are not limited to theobroma oil (also known as cocoa butter), glycerinated gelatin, other glycerides, hydrogenated vegetable oils, mixtures of polyethylene glycols of various molecular weights and fatty acid esters of polyethylene glycol. See, for example: Remington's Pharmaceutical Sciences, 16^{th} Ed., Mack Publishing, Easton, PA, 1980, pp. 1530-1533 for further discussion of suppository dosage forms.

In some embodiments a compound of Formula II, or a pharmaceutically acceptable salt, solvate and/or prodrug thereof, is coupled with soluble polymers as targetable drug carriers. Such polymers include, for example, polyvinylpyrrolidone, pyran copolymer, polyhydroxypropylmethacrylamide-phenol, polyhydroxy-ethylaspartamide-phenol, or polyethyleneoxide-polylysine substituted with palmitoyl residues. Furthermore, in some embodiments, a compound of Formula II, or a pharmaceutically acceptable salt, solvate and/or prodrug thereof, is coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and crosslinked or amphipathic block copolymers of hydrogels.

The compounds of Formula II, or a pharmaceutically acceptable salt, solvate and/or prodrug thereof, are amenable to administration with the air of nano-carrier systems, such as liposomes, micelles, nanoparticles, nano-emulsions, lipidic nano-systems and the like (see for example, Bhat, M. et al. Chem. And Phys. Of Lipids, 2021, 236, 105053). Accordingly the present application includes a composition comprising one or more compounds of Formula II, or a pharmaceutically acceptable salt, solvate and/or prodrug thereof, and one or more components of a nano-carrier system.

A compound of Formula II, or a pharmaceutically acceptable salt, solvate and/or prodrug thereof, thereof is suitably used on its own but will generally be administered in the form of a pharmaceutical composition in which the one or more compounds (the active ingredient) is in association with a pharmaceutically acceptable carrier. Depending on the mode of administration, the pharmaceutical composition will comprise from about 0.05 wt % to about 99 wt % or about 0.10 wt % to about 70 wt %, of the active ingredient and from about 1 wt % to about 99.95 wt % or about 30 wt % to about 99.90 wt % of a pharmaceutically acceptable carrier, all percentages by weight being based on the total composition.

In some embodiments, the compound of Formula II, or a pharmaceutically acceptable salt, solvate and/or prodrug thereof, are used are administered in a composition comprising an additional therapeutic agent. Therefore the present application also includes a pharmaceutical composition comprising one of more compounds of Formula II, or a pharmaceutically acceptable salt, solvate and/or prodrug thereof, and an additional therapeutic agent, and optionally one or more pharmaceutically acceptable excipients. In some embodiments, the additional therapeutic agent is another known agent useful for treatment of a disease, disorder or condition by activation of a serotonin receptor, for example those listed in the Methods and Uses section above for the compounds of the application. In some embodiments, the additional therapeutic agent is a psychoactive drug.

In some embodiments, effective amounts of the one or more compounds of Formula II, or a pharmaceutically acceptable salt, solvate and/or prodrug thereof, vary according to factors such as the disease state, age, sex and/or weight of the subject or species. In some embodiments, the amount of a given compound or compounds that will correspond to an effective amount will vary depending upon factors, such as the given drug(s) or compound(s), the pharmaceutical formulation, the route of administration, the type of condition, disease or disorder, the identity of the subject being treated and the like, but can nevertheless be routinely determined by one skilled in the art.

In some embodiment, the compounds of Formula II, or a pharmaceutically acceptable salt, solvate and/or prodrug thereof, are administered one, two, three or four times a year. In some embodiments, the compounds of Formula II, or a pharmaceutically acceptable salt, solvate and/or prodrug thereof, are administered at least once a week. However, in another embodiment, the compounds of Formula II, or a pharmaceutically acceptable salt, solvate and/or prodrug thereof, are administered to the subject from about one time per two weeks, three weeks or one month. In another embodiment, the compounds of Formula II, or a pharmaceutically acceptable salt, solvate and/or prodrug thereof, are administered about one time per week to about once daily. In another embodiment, the compounds of Formula II, or a pharmaceutically acceptable salt, solvate and/or prodrug thereof, are administered 1, 2, 3, 4, 5 or 6 times daily. The length of the treatment period depends on a variety of factors, such as the severity of the disease, disorder or condition, the age of the subject, the concentration and/or the activity of the compounds of Formula II, or a pharmaceutically acceptable salt, solvate and/or prodrug thereof, and/or a combination thereof. It will also be appreciated that the effective dosage of the compound of Formula II, or a pharmaceutically acceptable salt, solvate and/or prodrug thereof, used for the treatment may increase or decrease over the course of a particular treatment regime. Changes in dosage may result and become apparent by standard diagnostic assays known in the art. In some instances, chronic administration is required. For example, the compounds of Formula II, or a pharmaceutically acceptable salt, solvate and/or prodrug thereof, are administered to the subject in an amount and for duration sufficient to treat the subject.

In some embodiments, the compounds of Formula II, or a pharmaceutically acceptable salt, solvate and/or prodrug thereof, are administered at doses that are non-hallucinogenic or non-psychotomimetic and taken in conjunction with psychotherapy or therapy and may occur once, twice, three, or four times a year. However, in some embodiments, the compounds of Formula II, or a pharmaceutically acceptable salt, solvate and/or prodrug thereof, are administered to the subject once daily, once every two days, once every 3 days, once a week, once every two weeks, once a month, once every two months, or once every three months at doses that are not hallucinogenic or not psychotomimetic.

A compound of Formula II, or a pharmaceutically acceptable salt, solvate and/or prodrug thereof, is either used alone or in combination with other known agents useful for treating diseases, disorders or conditions by activation of a serotonin receptor. When used in combination with other known agents useful in treating diseases, disorders by activation of a serotonin receptor, it is an embodiment that a compound of Formula II, or a pharmaceutically acceptable salt, solvate and/or prodrug thereof, is administered contemporaneously with those agents. As used herein, “contemporaneous administration” of two substances to a subject means providing each of the two substances so that they are both active in the individual at the same time. The exact details of the administration will depend on the pharmacokinetics of the two substances in the presence of each other and can include administering the two substances within a few hours of each other, or even administering one substance within 24 hours of administration of the other, if the pharmacokinetics are suitable. Design of suitable dosing regimens is routine for one skilled in the art. In particular embodiments, two substances will be administered substantially simultaneously, i.e., within minutes of each other, or in a single composition that contains both substances. It is a further embodiment of the present application that a combination of agents is administered to a subject in a non-contemporaneous fashion. In some embodiments, a compound of Formula II, or a pharmaceutically acceptable salt, solvate and/or prodrug thereof, is administered with another therapeutic agent simultaneously or sequentially in separate unit dosage forms or together in a single unit dosage form. Accordingly, the present application provides a single unit dosage form comprising one or more compounds of Formula II, or a pharmaceutically acceptable salt, solvate and/or prodrug thereof, an additional therapeutic agent and a pharmaceutically acceptable carrier.

The dosage of a compound of Formula II, or a pharmaceutically acceptable salt, solvate and/or prodrug thereof, varies depending on many factors such as the pharmacodynamic properties of the compound, the mode of administration, the age, health and weight of the recipient, the nature and extent of the symptoms, the frequency of the treatment and the type of concurrent treatment, if any and the clearance rate of the compound in the subject to be treated. One of skill in the art can determine the appropriate dosage based on the above factors. In some embodiments, one or more compounds of Formula II, or a pharmaceutically acceptable salt, solvate and/or prodrug thereof, are administered initially in a suitable dosage that is adjusted as required, depending on the clinical response.

In some embodiments, dosages will generally be selected to maintain a serum level of the one or more compounds of the application from about 0.01 μg/cc to about 1000 μg/cc, or about 0.1 μg/cc to about 100 μg/cc. As a representative example, oral dosages of one or more compounds of Formula II will range between about 10 μg per day to about 1000 mg per day for an adult, suitably about 10 μg per day to about 500 mg per day, more suitably about 10 μg per day to about 500 mg per day. For parenteral administration, a representative amount is from about 0.0001 mg/kg to about 10 mg/kg, about 0.001 mg/kg to about 100 mg/kg, about 0.1 mg/kg to about 1000 mg/kg or about 1 mg/kg to about 10000 mg/kg will be administered. For oral administration, a representative amount is from about 0.001 μg/kg to about 10 mg/kg, about 0.1 μg/kg to about 100 mg/kg, about 0.01 μg/kg to about 1000 mg/kg or about 1 μg/kg to about 10000 mg/kg. For administration in suppository form, a representative amount is from about 0.1 mg/kg to about 10 mg/kg or about 0.1 mg/kg to about 100 mg/kg.

In some embodiments, dosages will generally be selected to maintain a serum level of the one or more compounds of Formula II, or a pharmaceutically acceptable salt, solvate and/or prodrug thereof, from about 0.01 μg/cc to about 1000 μg/cc, or about 0.1 μg/cc to about 100 μg/cc. As a representative example, oral dosages of one or more compounds of Formula II, or a pharmaceutically acceptable salt, solvate and/or prodrug thereof, will range between about 10 μg per day to about 1000 mg per day for an adult, suitably about 10 μg per day to about 500 mg per day, more suitably about 10 μg per day to about 200 mg per day. For parenteral administration, a representative amount is from about 0.0001 mg/kg to about 10 mg/kg, about 0.0001 mg/kg to about 1 mg/kg, about 0.01 mg/kg to about 0.1 mg/kg or about 0.0001 mg/kg to about 0.01 mg/kg will be administered. For oral administration, a representative amount is from about 0.001 μg/kg to about 10 mg/kg, about 0.1 μg/kg to about 10 mg/kg, about 0.01 μg/kg to about 1 mg/kg or about 0.1 μg/kg to about 1 mg/kg. For administration in suppository form, a representative amount is from about 0.1 mg/kg to about 10 mg/kg or about 0.1 mg/kg to about 1 mg/kg. In some embodiments of the application, compositions are formulated for oral administration and the one or more compounds are suitably in the form of tablets containing 0.1, 0.25, 0.5, 0.75, 1.0, 5.0, 10.0, 20.0, 25.0, 30.0, 40.0, 50.0, 60.0, 70.0, 75.0, 80.0, 90.0, 100.0, 150, 200, 250, 300, 350, 400, 450, 500, 550, 600, 650, 700, 750, 800, 850, 900, 950 or 1000 mg of active ingredient (one or more compounds of Formula II, or a pharmaceutically acceptable salt, solvate and/or prodrug thereof) per tablet. In some embodiments of the application the compounds of Formula II, or a pharmaceutically acceptable salt, solvate and/or prodrug thereof, are administered in a single daily, weekly or monthly dose or the total daily dose is divided into two, three or four daily doses.

In some embodiments, the compounds of Formula II, or a pharmaceutically acceptable salt, solvate and/or prodrug thereof, are used or administered in an effective amount which comprises administration of doses or dosage regimens that are devoid of clinically meaningful psychedelic/psychotomimetic actions. In some embodiments, the compounds of Formula II, or a pharmaceutically acceptable salt, solvate and/or prodrug thereof, are used or administered in an effective amount which comprises administration of doses or dosage regimens that provide clinical effects similar to those exhibited by a human plasma psilocin Cmax of 4 ng/mL or less and/or human 5-HT_{2A} human CNS receptor occupancy of 40% or less or those exhibited by a human plasma psilocin Cmax of 1 ng/mL or less and/or human 5-HT_{2A} human CNS receptor occupancy of 30% or less.

In some embodiments, the compounds of Formula II are used or administered in an effective amount which comprises administration of doses or dosage regimens that provide clinical effects similar to those exhibited by a human plasma psilocin Cmax of 4 ng/mL or greater and/or human 5-HT_{2A} human CNS receptor occupancy of 40% or greater or those exhibited by a human plasma psilocin Cmax of 10 ng/mL or greater and/or human 5-HT_{2A} human CNS receptor occupancy of 60% or greater.

In some embodiments, the compounds of Formula II, or a pharmaceutically acceptable salt, solvate and/or prodrug thereof, are used or administered in an effective amount which comprises administration of doses or dosage regimens that provide clinical effects similar to those exhibited by a human plasma psilocin Tmax in excess of 60 minutes, in excess of 120 minutes or in excess of 180 minutes.

To be clear, in the above, the term “a compound” also includes embodiments wherein one or more compounds of Formula II, or a pharmaceutically acceptable salt, solvate, and/or prodrug thereof, are referenced. Likewise, the term “compounds” also includes embodiments wherein only one compound of Formula II, or a pharmaceutically acceptable salt, solvate and/or prodrug thereof, is referenced.

### VI. Preparation of Compounds

Compounds of the present application can be prepared by various synthetic processes. The choice of particular structural features and/or substituents may influence the selection of one process over another. The selection of a particular process to prepare a given compound of the application is within the purview of the person of skill in the art. Some starting materials for preparing compounds of the present application are available from commercial chemical sources or may be extracted from cells, plants, animals or fungi. Other starting materials, for example as described below, are readily prepared from available precursors using straightforward transformations that are well known in the art. In the Schemes below showing some embodiments of methods of preparation of compounds of the application, all variables are as defined in Formula I, unless otherwise stated.

In some embodiments, the compounds of Formula I wherein R^{2′} and R^{2″} are H or D are prepared by reduction of the corresponding indole compound A, wherein R¹, R², R³, R⁴, R⁵ and R⁶ are as defined in Formula I, as shown in Scheme 1

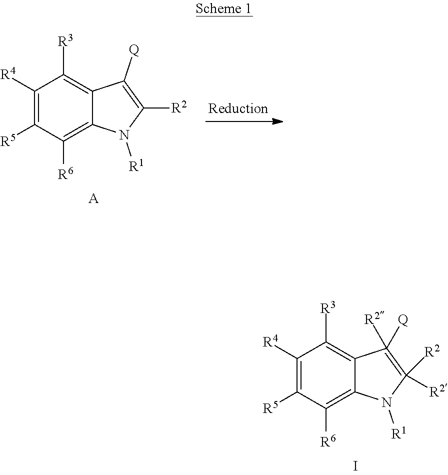

In some embodiments the reduction is performed using a silane reducing agent, such as triethyl silane in an acid such as trifluoroacetic acid, or deuterated versions thereof. In some embodiments, the reaction is made enantio- or diastereoselective via the use of chiral reducing agents or chiral auxiliaries. In some embodiments, the reduction is performed using any suitable reducing conditions known in the art, such as, for example, the reducing conditions described in He, Yi; et al: Tetrahedron Letters (2014), 55(29), 3938-3941 and Somei, Masanori; et al: Heterocycles (1995), 40(1), 119-22

In some embodiments, compounds of Formula A are available as shown in Scheme 2:

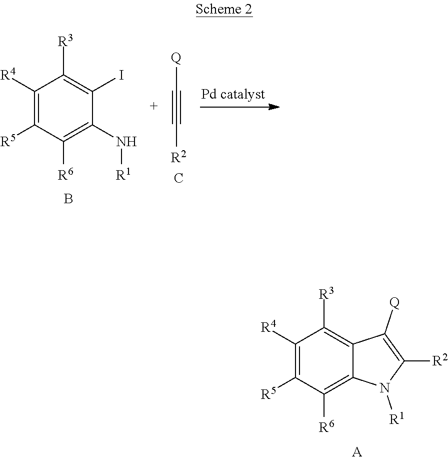

Therefore, in some embodiments, ortho-iodoaniline compounds of Formula B, wherein R¹ and R³-R⁶ are as defined in Formula I, are reacted with alkynes of Formula C, wherein R² and Q are as defined in Formula I, in the presence of a palladium catalyst, for example using the conditions described in Fricke et al., Chem. Eur. J., 2019, 25(4):897-903], to provide the compounds of Formula A. A person skilled in the art would appreciate that the reaction shown in Scheme 2 can also be used to provide compounds of Formula A by using a compound of Formula B wherein R¹ is a suitable protecting group and removing the protecting group to provide the compound of Formula A.

In some embodiments, when Q is

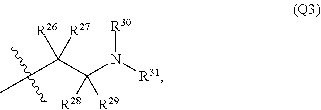

and R²⁶-R³¹ are either H or D, the compounds of Formula A are prepared as shown in Scheme 3:

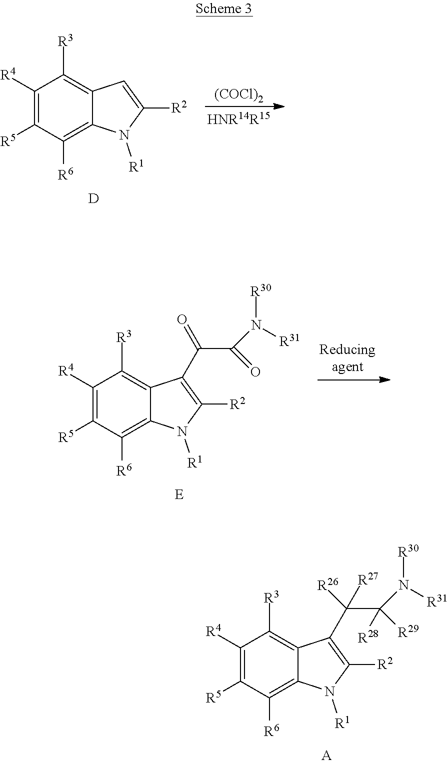

Therefore, in some embodiments, compounds of Formula D, wherein R¹-R⁶ are as defined in Formula I, are reacted with oxalyl chloride followed by an amine NHR³⁰R³¹, wherein R³⁰ and R³¹ are as defined in Formula I, to provide compounds of Formula E. Subsequent Al-based reduction of the compounds of Formula E, for example in the presence of lithium borohydride, lithium aluminum hydride or lithium aluminum deuteride, provides compounds of Formula A, wherein Q is

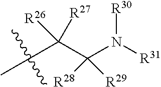

and R²⁶-R²⁷ are either H or D, R¹-R⁶ and R³⁰-R³¹ are as defined in Formula I.

In some embodiments, when Q is

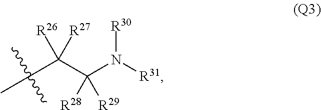

R⁴ is an alkoxy (including, for example, C₁₋₆alkoxy or O-A), R¹, R², R³, R⁵, R⁶, and R²⁶-R³¹ are as defined in Formula I, the compounds of Formula A are prepared as shown in Scheme 4:

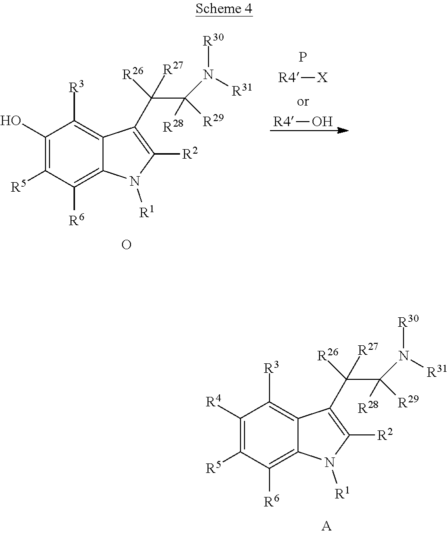

Therefore, in some embodiments, compounds of Formula O, wherein R⁴ is OH and A, R³, R⁵, R⁶, and R²⁶-R³¹ are as defined in Formula I are reacted with a compound of Formula P wherein X is a suitable leaving group such as I or Br and R⁴′ is alkyl or A as defined in Formula I, under suitable conditions such as in the presence of a base such a potassium carbonate and in a suitable solvent such as acetone to provide compounds of Formula A. In some embodiments, compounds of Formula O, wherein R⁴ is OH and A, and R¹, R², R³, R⁵, R⁶, and R²⁶-R³¹ are as defined in Formula I, are reacted with a suitable alcohol under suitable Mitsunobu reaction conditions, such as in the presence of a diethyl azodicarboxylate (DEAD) and triphenylphosphine. In some embodiments, the suitable conditions are any suitable conditions known in the art, such as, for example, the coupling conditions described in WO2005009958A1 and U.S. Pat. No. 6,133,287.

A person skilled in the art would appreciate that a similar process as described in Scheme 8 is used to provide compounds of Formula I wherein any one of R³, R⁵, R⁵ and R⁶ is an alkoxy.

Compounds of Formula D or A, wherein one or more of R², R³-R⁶ are deuterium are available, for example, using a hydrogen-deuterium exchange reaction on a suitable starting substrate, wherein this exchange reaction is catalyzed by Pd/C in D₂O as described in Esaki, H. et al. Tetrahedron, 2006, 62:10954-10961, and modifications thereof known to a person skilled in the art.

Compounds of Formula A wherein R⁴ is OCD₃ are available, for example, using methods as described in Xu, Y-Z and Chen, C. J. Label Compd. Radiopharm. (2006) 49:897-902, and modifications thereof and modifications thereof known to a person skilled in the art.

Compounds of Formula B, C, D, and F, as well as other reagents used in the above Schemes, are available from commercial sources or can be readily prepared using methods known in the art.

A person skilled in the art would appreciate that further manipulation of the substituent groups using known chemistry can be performed on the intermediates and final compounds in the Schemes above to provide alternative compounds of the application.

Nucleophilic displacement reaction conditions comprise any known method for the reaction of a nucleophile to displace a leaving group to form a bond that is compatible with the intermediates and products shown in the above Schemes or that may be used to prepare a compound of Formula I and II, or a pharmaceutically acceptable salt, solvate, zwitterion and/or prodrug thereof. In some embodiments, such conditions comprise combining reactants in the presence of a base in a suitable solvent.

Salts of compounds of the application may be formed by methods known to those of ordinary skill in the art, for example, by reacting a compound of the application with an amount of acid or base, such as an equivalent amount, in a medium such as one in which the salt precipitates or in aqueous medium followed by lyophilization.

The formation of solvates will vary depending on the compound and the solvate. In general, solvates are formed by dissolving the compound in the appropriate solvent and isolating the solvate by cooling or using an antisolvent. The solvate is typically dried or azeotroped under ambient conditions. The selection of suitable conditions to form a particular solvate can be made by a person skilled in the art. Examples of suitable solvents are ethanol, water and the like. When water is the solvent, the molecule is referred to as a “hydrate”. The formation of solvates of the compounds of the application will vary depending on the compound and the solvate. In general, solvates are formed by dissolving the compound in the appropriate solvent and isolating the solvate by cooling or using an antisolvent. The solvate is typically dried or azeotroped under ambient conditions. The selection of suitable conditions to form a particular solvate can be made by a person skilled in the art.

Prodrugs of the compounds of the present application may be, for example, conventional esters formed with available hydroxy, thiol, amino or carboxyl groups. For example, available hydroxy or amino groups may be acylated using an activated acid in the presence of a base, and optionally, in inert solvent (e.g. an acid chloride in pyridine).

Isotopically-enriched compounds of the application and pharmaceutically acceptable salts and/or solvates thereof, can be prepared without undue experimentation by conventional techniques well known to those skilled in the art or by processes analogous to those described in the Schemes and Examples herein using suitable isotopically-enriched reagents and/or intermediates.

Throughout the processes described herein it is to be understood that, where appropriate, suitable protecting groups will be added to and subsequently removed from, the various reactants and intermediates in a manner that will be readily understood by one skilled in the art. Conventional procedures for using such protecting groups as well as examples of suitable protecting groups are described, for example, in “Protective Groups in Organic Synthesis”, T. W. Green, P. G. M. Wuts, Wiley-Interscience, New York, (1999). It is also to be understood that a transformation of a group or substituent into another group or substituent by chemical manipulation can be conducted on any intermediate or final product on the synthetic path toward the final product, in which the possible type of transformation is limited only by inherent incompatibility of other functionalities carried by the molecule at that stage to the conditions or reagents employed in the transformation. Such inherent incompatibilities and ways to circumvent them by carrying out appropriate transformations and synthetic steps in a suitable order, will be readily understood to one skilled in the art. Examples of transformations are given herein and it is to be understood that the described transformations are not limited only to the generic groups or substituents for which the transformations are exemplified. References and descriptions of other suitable transformations are given in “Comprehensive Organic Transformations—A Guide to Functional Group Preparations” R. C. Larock, VHC Publishers, Inc. (1989). References and descriptions of other suitable reactions are described in textbooks of organic chemistry, for example, “Advanced Organic Chemistry”, March, 4th ed. McGraw Hill (1992) or, “Organic Synthesis”, Smith, McGraw Hill, (1994). Techniques for purification of intermediates and final products include, for example, straight and reversed phase chromatography on column or rotating plate, recrystallisation, distillation and liquid-liquid or solid-liquid extraction, which will be readily understood by one skilled in the art.

It is also to be understood that a transformation of a group or substituent into another group or substituent by chemical manipulation can be conducted on any intermediate or final product on the synthetic path toward the final product, in which the possible type of transformation is limited only by inherent incompatibility of other functionalities carried by the molecule at that stage to the conditions or reagents employed in the transformation. Such inherent incompatibilities, and ways to circumvent them by carrying out appropriate transformations and synthetic steps in a suitable order, will be readily understood to one skilled in the art. Examples of transformations are given herein, and it is to be understood that the described transformations are not limited only to the generic groups or substituents for which the transformations are exemplified. References and descriptions of other suitable transformations are given in “Comprehensive Organic Transformations—A Guide to Functional Group Preparations” R. C. Larock, VHC Publishers, Inc. (1989). References and descriptions of other suitable reactions are described in textbooks of organic chemistry, for example, “Advanced Organic Chemistry”, March, 4th ed. McGraw Hill (1992) or, “Organic Synthesis”, Smith, McGraw Hill, (1994).

Techniques for purification of intermediates and final products include, for example, straight and reversed phase chromatography on column or rotating plate, recrystallisation, distillation and liquid-liquid or solid-liquid extraction, which will be readily understood by one skilled in the art.

The products of the processes of the application may be isolated according to known methods, for example, the compounds may be isolated by evaporation of the solvent, by filtration, centrifugation, chromatography or other suitable method.

One skilled in the art will recognize that where a reaction step of the present application is carried out in a variety of solvents or solvent systems, said reaction step may also be carried out in a mixture of the suitable solvents or solvent systems.

A person skilled in the art would appreciate that that same methods of preparation described above for the compounds of the application can be used to prepare compounds of Formula II, or a pharmaceutically acceptable salt, solvate and/or prodrug thereof.

### EXAMPLES

### A. Chemical Synthesis

### General Methods

All starting materials used herein were commercially available or earlier described in the literature. The ¹H and ¹³C NMR spectra were recorded either on Bruker 300, Bruker DPX400 or Varian +400 spectrometers operating at 300, 400 and 400 MHz for ¹H NMR respectively, using TMS or the residual solvent signal as an internal reference, in deuterated chloroform as solvent unless otherwise indicated. All reported chemical shifts are in ppm on the delta-scale, and the fine splitting of the signals as appearing in the recordings is generally indicated, for example as s: singlet, br s: broad singlet, d: doublet, t: triplet, q: quartet, m: multiplet. Unless otherwise indicated, in the tables below, ¹H NMR data was obtained at 400 MHz, using CDCl₃ as the solvent.

Purification of products was carried out using Chem Elut Extraction Columns (Varian, cat #1219-8002), Mega BE-SI (Bond Elut Silica) SPE Columns (Varian, cat #12256018; 12256026; 12256034) or by flash chromatography in silica-filled glass columns.

### Example 1: 2-(5-Methoxyindolin-3-yl)-N,N-dimethylethan-1-amine (II-1)

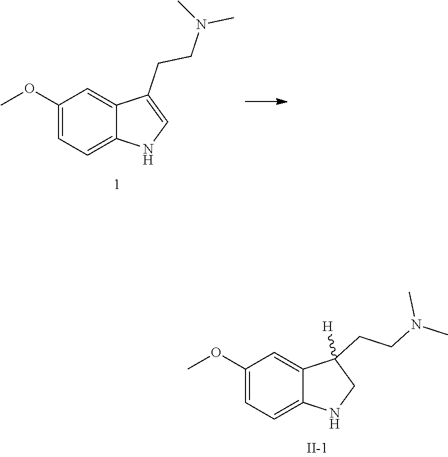

Synthesis of 2-(5-methoxyindolin-3-yl)-N,N-dimethylethan-1-amine (2): A solution of 2-(5-methoxy-1H-indol-3-yl)-N,N-dimethylethan-1-amine (0.4 g, 1.832 mmol) in trifluoroacetic acid (30 mL) was treated with triethyl silane (0.58 mL, 3.664 mmol) and the resulting solution was stirred at 50° C. for overnight (16 h). The reaction was brought to room temperature and solvent was evaporated. Crude was basified with 4 N NaOH solution and product was extracted into CH₂Cl₂ (3×50 mL). Combined CH₂Cl₂ layer was dried (Na₂SO₄) and solvent was evaporated to obtain crude product as brown liquid. Crude was purified by column chromatography (2 M NH₃ in MeOH: CH₂Cl₂, 5:95) on silica gel to obtain the title compound II-1 (0.31 g, 77.5%) as light brown oil. ¹H NMR (CDCl₃): δ 6.76 (s, 1H), 6.65-6.60 (m, 2H), 3.78 (s, 3H), 3.70 (t, 1H, J=6.0 Hz), 3.35-3.20 (m, 2H), 2.43-2.32 (m, 3H), 2.28 (s, 6H), 2.05-1.99 (m, 1H), 1.78-1.71 (m, 1H); ESI-MS (m/z, %): 221 (MH⁺, 100).

### Example 2: 2-(Indolin-3-yl)-N,N-dimethylethan-1-amine (II-2)]

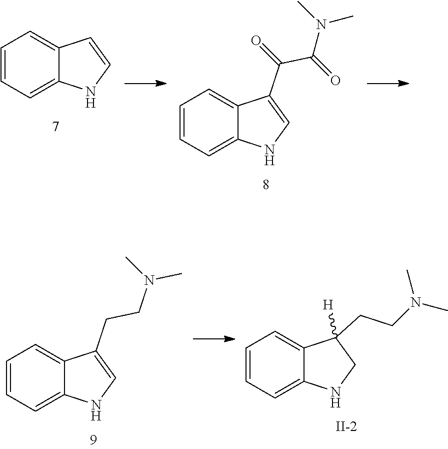 [SMILES: c1ccc2[nH]ccc2c1 ; CN(C)C(=O)C(=O)c1c[nH]c2ccccc12 ; CN(C)CCc1c[nH]c2ccccc12 ; CN(C)CCC1CNc2ccccc21]

Synthesis of 2-(1H-indol-3-yl)-N,N-dimethyl-2-oxoacetamide (8): A solution of 1H-indole (5.0 g, 42.698 mmol) in dry THF (50 mL) was treated with oxalyl chloride (3.61 mL, 42.698 mmol) at 0° C. over a period of 5 min. The reaction was brought to room temperature and stirred for 3 h. The reaction was treated with dimethylamine (85 mL, 170.0 mmol, 2 M solution in THF) over a period of 15 min. and stirred for overnight (16 h). The reaction was quenched with water (100 mL) and product was extracted into ethyl acetate (2×100 mL). Combined ethyl acetate layer was washed with brine (50 mL) and dried (Na₂SO₄). Solvent was evaporated and crude was purified by crystallization from ethyl acetate: Hexanes (1:1) to obtain the title compound 8 (7.88 g, 85.4%) as a beige solid. ¹H NMR (DMSO-d₆): δ 12.28 (brs, 1H), 8.11-8.09 (m, 2H), 7.53 (dd, 1H, J=3.0 Hz), 7.30-7.23 (m, 2H), 2.99 (s, 3H), 2.91 (s, 3H); ESI-MS (m/z, %): 238 (M+Na, 100).

Synthesis of 2-(1H-indol-3-yl)-N,N-dimethylethan-1-amine (9): A suspension of lithium aluminum hydride (2.80 g, 74.0 mmol) in dry THF (25 mL) was treated with 2-(1H-indol-3-yl)-N,N-dimethyl-2-oxoacetamide (2.0 g, 9.248 mmol) in dry THF (25 mL) at 0° C. over a period of 10 min. The reaction was brought to room temperature, then refluxed for additional 16 hours. The reaction was worked-up and purified as described for compound 5 to obtain the title compound 9 (1.7 g, quantitative) as an off-white solid. ¹H NMR (DMSO-d₆): δ 10.76 (s, 1H), 7.50 (d, 1H, J=3.0 Hz), 7.32 (d, 1H, J=6.0 Hz), 7.13 (s, 1H), 7.07-7.03 (m, 1H), 6.98-6.95 (m, 1H), 2.82-2.79 (m, 2H), 2.52-2.49 (m, 2H), 2.21 (s, 6H); ESI-MS (m/z, %): 189 (MH⁺, 100).

Synthesis of 2-(indolin-3-yl)-N,N-dimethylethan-1-amine (11-2): A solution of 2-(1H-indol-3-yl)-N,N-dimethylethan-1-amine (1.47 g, 7.807 mmol) in trifluoroacetic acid (60 mL) was treated with triethyl silane (2.49 mL, 15.615 mmol) and the resulting solution was stirred at 50° C. for overnight (16 h). The reaction was worked-up and purified as described for compound II-1 to obtain the title compound II-2 (1.1 g, 74.3%) as a pale-yellow oil. ¹H NMR (DMSO-d₆): δ 7.01 (d, 1H, J=6.0 Hz), 6.90 (t, 1H, J=6.0 Hz), 6.54 (dd, 1H, J=1.5, 6.0 Hz), 6.48 (d, 1H, J=6.0 Hz), 5.41 (s, 1H), 3.55-3.50 (m, 1H), 3.20-3.13 (m, 1H), 3.08-3.03 (m, 1H), 2.31-2.20 (m, 2H), 2.22 (s, 6H), 1.90-1.84 (m, 1H), 1.59-1.50 (m, 1H); ESI-MS (m/z, %): 191 (MH⁺, 100).

### Example 3: 2-(5-Fluoroindolin-3-yl)-N,N-dimethylethan-1-amine (1-2)]

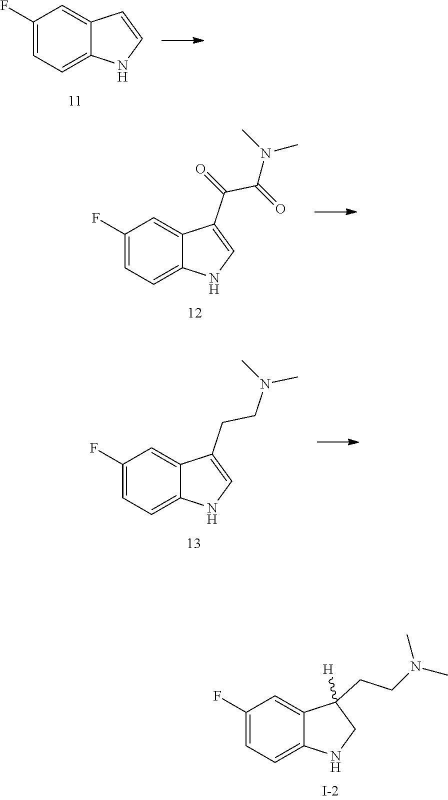 [SMILES: Fc1ccc2[nH]ccc2c1 ; CN(C)C(=O)C(=O)c1c[nH]c2ccc(F)cc12 ; CN(C)CCc1c[nH]c2ccc(F)cc12 ; CN(C)CCC1CNc2ccc(F)cc21]

Synthesis of 2-(5-fluoro-1H-indol-3-yl)-N,N-dimethyl-2-oxoacetamide (12): A solution of 5-fluoro-1H-indole (5.0 g, 36.999 mmol) in dry THF (50 mL) was treated with oxalyl chloride (3.11 mL, 36.999 mmol) at 0° C. over a period of 5 min. The reaction was brought to room temperature and stirred for 3 h. The reaction was treated with dimethylamine (74 mL, 147.996 mmol, 2 M solution in THF) over a period of 15 min. and stirred for overnight (16 h). The reaction was worked-up and purified as described for compound 8 to obtain the title compound 12 (7.12 g, 82.2%) as a light-yellow solid. ¹H NMR (DMSO-d₆): δ 12.40 (brs, 1H), 8.17 (s, 1H), 7.76 (dd, 1H, J=3.0, 4.5 Hz), 7.55 (dd, 1H, J=3.0, 6.0 Hz), 7.16-7.12 (m, 1H), 2.99 (s, 3H), 2.91 (s, 3H); ESI-MS (m/z, %): 256 (M+Na, 100).

Synthesis of 2-(5-fluoro-1H-indol-3-yl)-N,N-dimethylethan-1-amine (13): A suspension of lithium aluminum hydride (2.59 g, 68.308 mmol) in dry THF (25 mL) was treated with 2-(5-fluoro-1H-indol-3-yl)-N,N-dimethyl-2-oxoacetamide (2.0 g, 8.538 mmol) in dry THF (25 mL) at 0° C. over a period of 10 min. The reaction was brought to room temperature, then refluxed for additional 16 hours. The reaction was worked-up and purified as described for compound 5 to obtain the title compound 13 (1.7 g, quantitative) as a tan solid. ¹H NMR (DMSO-d₆): δ 10.87 (s, 1H), 7.30 (dd, 1H, J=3.0, 6.0 Hz), 7.24 (dd, 1H, J=3.0, 6.0 Hz), 7.21 (d, 1H, J=3.0 Hz), 6.90-6.86 (m, 1H), 2.78-2.75 (m, 2H), 2.52-2.47 (m, 2H), 2.20 (s, 6H); ESI-MS (m/z, %): 207 (MH⁺, 100).

Synthesis of 2-(5fluoroindolin-3-yl)-N,N-dimethylethan-1-amine (1-2): A solution of 2-(5-fluoro-1H-indol-3-yl)-N,N-dimethylethan-1-amine (1.51 g, 7.320 mmol) in trifluoroacetic acid (60 mL) was treated with triethyl silane (2.33 mL, 14.641 mmol) and the resulting solution was stirred at 50° C. for overnight (16 h). The reaction was worked-up and purified as described for compound II-1 to obtain the title compound I-2 (0.8 g, 52.6%) as a light brown oil. ¹H NMR (DMSO-d₆): δ 6.90-6.87 (m, 1H), 6.74-6.69 (m, 1H), 6.45-6.42 (m, 1H), 5.32 (s, 1H), 3.56-3.51 (m, 1H), 3.19-3.05 (m, 2H), 2.30-2.18 (m, 2H), 2.14 (s, 6H), 1.92-1.86 (m, 1H), 1.58-1.51 (m, 1H); ESI-MS (m/z, %): 209 (MH⁺, 100).

### Example 4: 2-(5-(Methoxy-d3)indolin-3-yl)-N,N-dimethylethan-1-amine (I-11)]

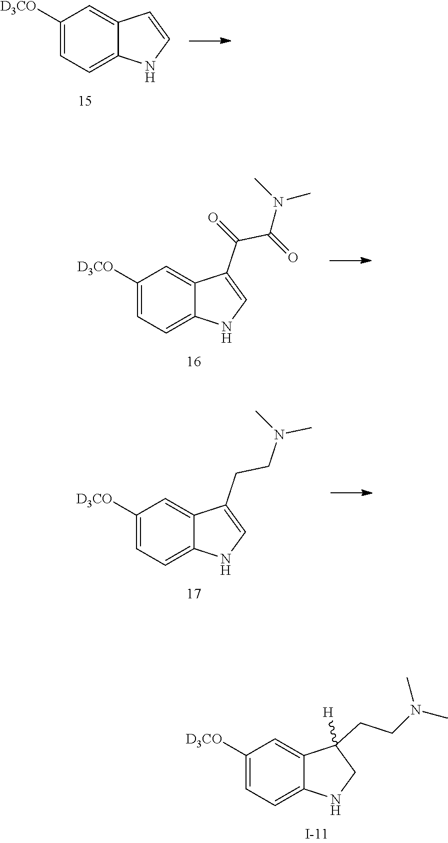

Synthesis of 2-(5-(methoxy-d₃)-1H-indol-3-yl)-N,N-dimethyl-2-oxoacetamide (16): A solution of 5-(methoxy-d₃)-1H-indole (2.5 g, 16.646 mmol) in dry THF (25 mL) was treated with oxalyl chloride (1.40 mL, 16.646 mmol) at 0° C. over a period of 5 min. The reaction was brought to room temperature and stirred for 3 h. The reaction was treated with dimethylamine (33.3 mL, 66.584 mmol, 2 M solution in THF) over a period of 15 min. and stirred for overnight (16 h). The reaction was worked-up and purified as described for compound 8 to obtain the title compound 1-11 (3.44 g, 82.9%) as an off-white solid. ¹H NMR (DMSO-d₆): δ 12.17 (s, 1H), 8.01 (s, 1H), 7.59 (s, 1H), 7.42 (d, 1H, J=6.0 Hz), 6.90 (dd, 1H, J=3.0, 4.5 Hz), 2.98 (s, 3H), 2.91 (s, 3H); ESI-MS (m/z, %): 272 (M+Na), 250 (MH⁺).

Synthesis of 2-(5-(methoxy-d₃)-1H-indol-3-yl)-N,N-dimethylethan-1-amine (17): A suspension of lithium aluminum hydride (2.43 g, 64.185 mmol) in dry THF (25 mL) was treated with 2-(5-(methoxy-d₃)-1H-indol-3-yl)-N,N-dimethyl-2-oxoacetamide (2.0 g, 8.023 mmol) in dry THF (25 mL) at 0° C. over a period of 10 min. The reaction was brought to room temperature, then refluxed for additional 16 hours. The reaction was worked-up and purified as described for compound 5 to obtain the title compound 17 (1.7 g, quantitative) as a tan solid. ¹H NMR (DMSO-d₆): δ 10.58 (s, 1H), 7.21 (d, 1H, J=6.0 Hz), 7.08 (d, 1H, J=3.0 Hz), 6.95 (d, 1H, J=3.0 Hz), 6.70 (dd, 1H, J=3.0, 4.5 Hz), 2.78-2.75 (m, 2H), 2.52-2.48 (m, 2H), 2.21 (s, 6H); ESI-MS (m/z, %): 222 (MH⁺, 100).

Synthesis of 2-(5-(methoxy-d₃)indolin-3-yl)-N,N-dimethylethan-1-amine (I-11): A solution of 2-(5-(methoxy-d₃)-1H-indol-3-yl)-N,N-dimethylethan-1-amine (1.34 g, 6.054 mmol) in trifluoroacetic acid (60 mL) was treated with triethyl silane (1.93 mL, 12.109 mmol) and the resulting solution was stirred at 50° C. for overnight (16 h). The reaction was worked-up and purified as described for compound II-1 to obtain the title compound I-11 (1.1 g, 81.5%) as a light brown oil. ¹H NMR (DMSO-d₆): δ 6.68 (d, 1H, J=3.0 Hz), 6.53-6.50 (m, 1H), 6.42 (d, 1H, J=6.0 Hz), 5.02 (brs, 1H), 3.49 (t, 1H, J=6.0 Hz), 3.17-3.00 (m, 2H), 2.29-2.22 (m, 2H), 2.15 (s, 6H), 1.91-1.86 (m, 1H), 1.55-1.48 (m, 1H); ESI-MS (m/z, %): 224 (MH⁺, 100).

### Example 5: 2-(Indolin-3-yl-4,5,6,7-d₄)-N,N-dimethylethan-1-amine (I-24)

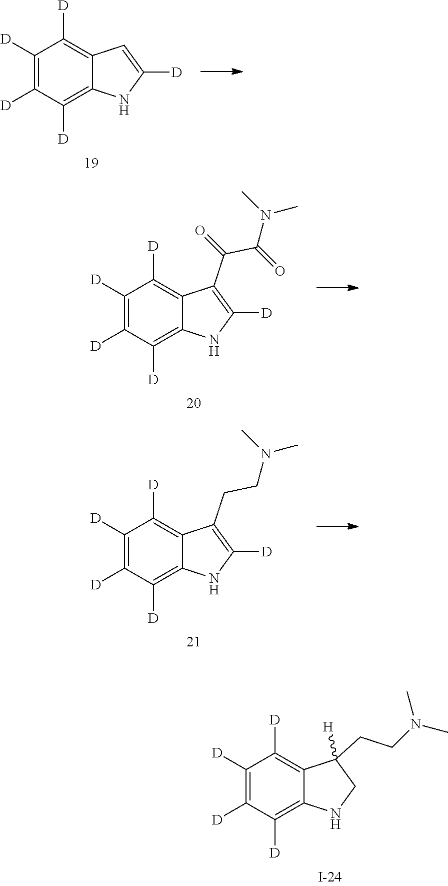 [SMILES: [2H]c1cc2c([2H])c([2H])c([2H])c([2H])c2[nH]1 ; [2H]c1[nH]c2c([2H])c([2H])c([2H])c([2H])c2c1C(=O)C(=O)N(C)C ; [2H]c1[nH]c2c([2H])c([2H])c([2H])c([2H])c2c1CCN(C)C ; [2H]c1c([2H])c([2H])c2c(c1[2H])NCC2CCN(C)C]

Synthesis of 2-(1H-indol-3-yl-2,4,5,6,7-d₅)-N,N-dimethyl-2-oxoacetamide (20): A solution of 1H-indole-2,3,4,5,6,7-d₆ (2.0 g, 16.236 mmol) in dry THF (25 mL) was treated with oxalyl chloride (1.37 mL, 16.236 mmol) at 0° C. over a period of 5 min. The reaction was brought to room temperature and stirred for 3 h. The reaction was treated with dimethylamine (32.4 mL, 64.944 mmol, 2 M solution in THF) over a period of 15 min. and stirred for overnight (16 h). The reaction was worked-up and purified as described for compound 8 to obtain the title compound 20 (3.0 g, 83.6%) as light brown solid. ¹H NMR (DMSO-d₆): δ 12.30 (s, 1H), 8.12-8.10 (m, 0.43H), 7.55-7.53 (m, 0.09H), 7.30-7.25 (m, 1.43H), 3.00 (s, 3H), 2.92 (s, 3H); ESI-MS (m/z, %): 244 (M+Na), 242 (100).

Synthesis of 2-(1H-indol-3-yl-2,4,5,6,7-d₅)-N,N-dimethylethan-1-amine (21): A suspension of lithium aluminum hydride (2.74 g, 72.310 mmol) in dry THF (25 mL) was treated with 2-(1H-indol-3-yl-2,4,5,6,7-d₅)-N,N-dimethyl-2-oxoacetamide (2.0 g, 9.039 mmol) in dry THF (25 mL) at 0° C. over a period of 10 min. The reaction was brought to room temperature, then refluxed for additional 16 hours. The reaction was worked-up and purified as described for compound 5 to obtain the title compound 21 (1.74 g, quantitative) as a tan solid. ¹H NMR (DMSO-d₆): δ 10.77 (s, 1H), 7.51-7.50 (m, 0.18H), 7.33 (s, 0.04H), 7.14 (d, 0.05H, J=1.5 Hz), 7.06 (t, 0.27H, J=3.0 Hz), 6.99-6.96 (m, 0.35H), 2.84-2.80 (m, 2H), 2.54-2.50 (m, 2H), 2.22 (s, 6H); ESI-MS (m/z, %): 194 (MH⁺), 193 (100).

Synthesis of 2-(indolin-3-yl-4,5,6,7-d₄)-N,N-dimethylethan-1-amine (1-24): A solution of 2-(1H-indol-3-yl-2,4,5,6,7-d₅)-N,N-dimethylethan-1-amine (1.2 g, 6.207 mmol) in trifluoroacetic acid (60 mL) was treated with triethyl silane (1.98 mL, 12.415 mmol) and the resulting solution was stirred at 50° C. for overnight (16 h). The reaction was worked-up and purified as described for compound II-1 to obtain the title compound 1-24 (1.0 g, 82.6%) as a light brown oil. ¹H NMR (DMSO-d₆): δ 7.00 (d, 0.32H, J=6.0 Hz), 6.90-6.88 (m, 0.41H), 6.53-6.46 (m, 0.88H), 5.38 (s, 1H), 3.53-3.50 (m, 1H), 3.17-3.13 (m, 1H), 3.07-3.03 (m, 1H), 2.31-2.20 (m, 2H), 2.14 (s, 6H), 1.88-1.83 (m, 1H), 1.57-1.51 (m, 1H); ESI-MS (m/z, %): 196 (MH⁺), 193 (100).

### Example 6: 2-(5,6-Dimethoxyindolin-3-yl)-N,N-dimethylethan-1-amine (1-33)

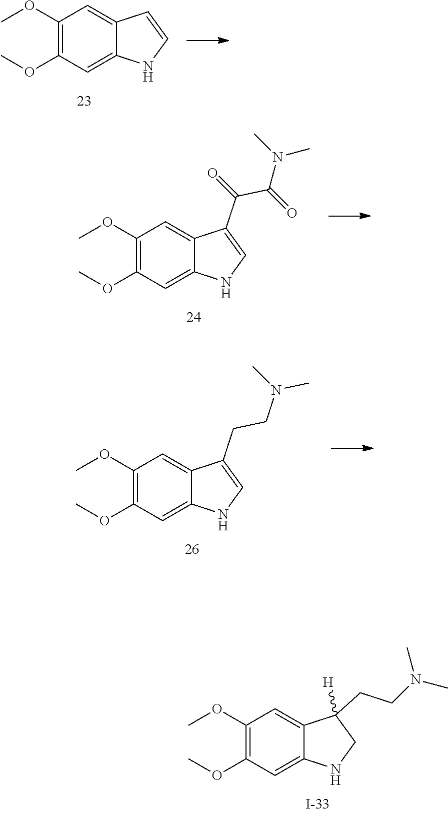

Synthesis of 2-(5,6-dimethoxy-1H-indol-3-yl)-N,N-dimethyl-2-oxoacetamide (24): A solution of 5,6-dimethoxy-1H-indole (2.0 g, 11.287 mmol) in dry THF (25 mL) was treated with oxalyl chloride (0.95 mL, 11.287 mmol) at 0° C. over a period of 5 min. The reaction was brought to room temperature and stirred for 3 h. The reaction was treated with dimethylamine (22.6 mL, 45.148 mmol, 2 M solution in THF) over a period of 15 min. and stirred for overnight (16 h). The reaction was worked-up and purified as described for compound 8 to obtain the title compound 24 (2.1 g, 67.5%) as light brown solid. ¹H NMR (DMSO-d₆): δ 12.02 (s, 1H), 7.89 (s, 1H), 7.58 (s, 1H), 7.04 (s, 1H), 3.81 (s, 6H), 2.99 (s, 3H), 2.92 (s, 3H); ESI-MS (m/z, %): 299 (M+Na, 100), 277 (MH⁺).

Synthesis of 2-(5,6-dimethoxy-1H-indol-3-yl)-N,N-dimethylethan-1-amine (25): A suspension of lithium aluminum hydride (2.2 g, 57.910 mmol) in dry THF (25 mL) was treated with 2-(5,6-dimethoxy-1H-indol-3-yl)-N,N-dimethyl-2-oxoacetamide (2.0 g, 7.239 mmol) in dry THF (25 mL) at 0° C. over a period of 10 min. The reaction was brought to room temperature, then refluxed for additional 16 hours. The reaction was worked-up and purified as described for compound 5 to obtain the title compound 25 (1.78 g, 99%) as a tan solid. ¹H NMR (DMSO-d₆): δ 10.44 (s, 1H), 6.98 (s, 1H), 6.95 (s, 1H), 6.87 (s, 1H), 3.76 (s, 6H), 2.77-2.74 (m, 2H), 2.52-2.48 (m, 2H), 2.22 (s, 6H); ESI-MS (m/z, %): 249 (MH⁺, 100).

Synthesis of 2-(5,6-dimethoxyindolin-3-yl)-N,N-dimethylethan-1-amine (1-33): A solution of 2-(5,6-dimethoxy-1H-indol-3-yl)-N,N-dimethylethan-1-amine (1.41 g, 5.678 mmol) in trifluoroacetic acid (60 mL) was treated with triethyl silane (1.81 mL, 11.356 mmol) and the resulting solution was stirred at 50° C. for overnight (16 h). The reaction was worked-up and purified as described for compound II-1 to obtain the title compound 1-33 (0.95 g, 67%) as a light brown oil. ¹H NMR (DMSO-d₆): δ 6.72 (s, 1H), 6.22 (s, 1H), 5.02 (s, 1H), 3.66 (s, 3H), 3.63 (s, 3H), 3.52-3.46 (m, 1H), 3.12-2.99 (m, 2H), 2.28-2.17 (m, 2H), 2.14 (s, 6H), 1.88-1.81 (m, 1H), 1.52-1.45 (m, 1H); ESI-MS (m/z, %): 251 (MH⁺, 100).

### Example 7: 2-(5-Methoxy-1-methylindolin-3-yl)-N,N-dimethylethan-1-amine (1-3)

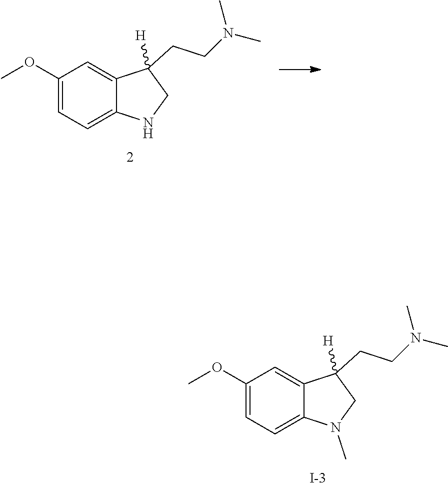

Synthesis of 2-(5-methoxy-1-methylindolin-3-yl)-N,N-dimethylethan-1-amine (48): A solution of 2-(5-methoxyindolin-3-yl)-N,N-dimethylethan-1-amine (1.2 g, 5.447 mmol) in dry CH₂Cl₂ (50 mL) was treated with acetic acid (0.93 mL, 16.341 mmol), followed by formaldehyde (1.21 mL, 16.341 mmol, 37% solution in water) at 0° C. The reaction was brought to room temperature and stirred for additional 6 h. The reaction was cooled to 0° C. and treated with sodium triacetoxy borohydride (3.46 g, 16.341 mmol). The reaction was brought to room temperature and stirred for overnight (16 h). The reaction was quenched with 4 N NaOH solution and product was extracted into dichloromethane (2×100 mL). Combined dichloromethane layer was dried (Na₂SO₄) and solvent was evaporated to obtain crude product. Crude was purified by column chromatography (2 M NH₃ in MeOH: CH₂Cl₂, 3:97) on silica gel to obtain the title compound 1-3 (0.9 g, 70.8%) as a brown liquid. ¹H NMR (DMSO-d₆): δ 6.70 (d, 1H, J=3.0 Hz), 6.60 (dd, 1H, J=3.0, 6.0 Hz), 6.41 (d, 1H, J=6.0 Hz), 3.65 (s, 3H), 3.10-3.04 (m, 1H), 2.78-2.74 (m, 1H), 2.60 (s, 3H), 2.31-2.21 (m, 3H), 2.15 (s, 6H), 1.93-1.87 (m, 1H), 1.58-1.48 (m, 1H); ESI-MS (m/z, %): 235 (MH⁺, 100).

### B. Biological Testing

### Example B1: FLIPR Assay: Human 5-HT_{2A}

### I. Assessment of the Effect of Exemplary Compounds of Formula I Targeting on Human 5-HT_{2A} (h5-HT_{2A}) Receptor Under Agonist Mode

### Compound Preparation and Assay Controls

### I.a. Reagent and Materials

| Regents | Vendor | Cat# |
|---|---|---|
| DMEM | Gibco | 10569010 |
| FBS | Hyclone | SH30406 |
| Penicillin-Streptomycin | Invitrogen | 15140 |
| Hygromycin B | Invivogen | Ant-hg-5 |
| G418 | Invitrogen | 11811031 |
| Tetracycline hydrochloride | Abcam | ab141223 |
| DPBS | Gibco | 14190250 |
| DMSO | Millipore | 1029312500 |
| Probenecid | Sigma | P8761 |
| FLIPR Calcium 6 Assay Kit | Molecular Device | R8191 |
| HEPES | Invitrogen | 15630 |
| Hank's Buffered Saline Solution | Invitrogen | 14025 |
| Serotonin HCl | Selleck | S4244 |

### I.b. Instrumentation and Consumables

| Item | Supplier | Cat# |
|---|---|---|
| Fluorometric Imaging Plate Reader | Molecular Device | Tetra |
| (FLIPR) | | |
| Countess Automated Cell Counter | Invitrogen | Countess |
| Cell Counting Chamber Slides | Invitrogen | C10312 |
| STERI-CYCLE CO₂ Incubator | Thermo | 371 |
| 1300 Series Class II Biological Safety | Thermo | 1389 |
| Cabinet | | |
| Table-type Large Capacity Low Speed | Cence | L550 |
| Centrifuge | | |
| Centrifuge | Eppendorf | 5702 |
| Echo | Labcyte | 550 |
| Echo | Labcyte | 655 |
| Electro-thermal incubator | Shanghai Yiheng | DHP-9031 |
| plate shaker | IKA | MS3 digital |
| Water Purification System | ULUPURE | UPH-III-20T |
| Versatile and Universal pH | Mettler Toledo | S220 |
| and Conductivity Meters | | |
| 384-Well plate | Corning | 356663 |
| 384-Well LDV Clear microplate | LABCYTE | LP-0200 |
| 384-Well Polypropylene microplate | LABCYTE | PP-0200 |
| 384-well compound plate | Corning | 3657 |
| T25 cell culture flask | Corning | 430639 |
| 50 mL Polypropylene Centrifuge Tube | JET | CFT011500 |
| 15 mL Polypropylene Centrifuge Tube | JET | CFT011150 |

### I.c. Experimental Methods and Procedures

1. Cells were cultured in cell culture medium (DMEM containing 10% FBS, 1×penicillin-streptomycin 300 μg/ml G418 and 100 μg/ml hygromycin B) at 37° C., 5% (v/v) CO2.
2. One day before the assays, the cells were detached using TrypLE™ Express and cells were counted using cell counter. Only cells with >85% viability were used for the assay.
3. 20000 cells/well were seeded in 30 μl/well culture medium to a 384-well cell plate and cells were incubated overnight at 37° C., 5% (v/v) CO2.
4. On the assay day, 2×dye solution was prepared following the manual of the FLIPR® Calcium 6 Assay Kit: i. The dye was diluted with assay buffer (20 mM HEPES in 1× HBSS, PH7.4); ii. Probenecid was added to the final concentration of 5 mM; iii. Vortex vigorously for 1-2 minutes.
5. Medium was removed from cell plate by flicking the cell plate on towel papers.
6. 10 μl of assay buffer and 10 μl of 2×dye solution was added to each well of the cell plate.
7. The cell plate was placed on plate shaker, the plate was agitated at 600 rpm for 2 minutes. The plate was incubated at 37° C. for 2 hours followed by additional 15-minute incubation at 25° C.
8. 3×compound in assay buffer was prepared: a. Reference compounds were diluted to required concentration with DMSO. The compounds were added to a 384-well compound plate; b. Serial dilutions were performed; c. 10 mM test compounds were added to the compound plate, and 3-fold serial dilutions were performed. d. Transfer 60 nl/well of compounds from source plate to a 384-well compound plate (Corning, 3657) by using an Echo; e. Add 20 μl/well assay buffer to the compound plate; f. Mix the plate on plate shaker for 2 mins;
9. The cell plate, compound plate and tips were put into FLIPR, 10 μl of 3× compound was transferred to the cell plate per well with FLIPR.

### Data Analysis

i. The normalized fluorescence reading (RFU) was calculated as shown below, where Fmax and Fmin stand for maximum and minimum of calcium signal during defined time window: RFU=Fmax−Fmin.
ii. Calculate the percentage activation using following equation:

$$\% Activation = \frac{( RFUcompound - RFU low control )}{( RFU top concentration of reference agonist - RFU low control )} \times 100 \%$$

Iii. Calculate EC₅₀ by fitting % activation against log of compound concentrations with Hill equation using XLfit.

### Results & Discussion

Exemplary compounds of Formula I were evaluated functionally using FLIPR assay for their effect on h5-HT_{2A} receptor under agonist mode. Table 2 shows the EC50 (nM) and RFU data for exemplary compounds of the application.

TABLE 2

5-HT2A (FLIPR)

| Compound ID# | EC₅₀(nM) | RFU@10 μM |
|---|---|---|
| 5-MeO-DMT | 88 | 2081 |
| DMT | 364 | 381 |
| Psilocin | 70/75 | 416/308 |
| II-1 | 202/274 | 1813/1180 |
| II-2 | 1225 | 544 |
| I-2 | 107 | 622 |
| I-11 | 19 | 980 |
| I-33 | ND | 286 |
| I-3 | 404 | 1014 |

ND: not detected

### Results & Discussion

Exemplary compound of Formula I and Formula II were evaluated functionally using FLIPR assay for their effect on h5-HT_{2A} receptor under agonist mode. EC₅₀ (nM) concentrations and their respective RFU at 10 μM are illustrated in Table 2. This assay confirms that the compounds of the application are effective agonists of the target human 5-HT2A receptors.

### II. Human 5-HT_{2A}: Radioligand Binding Assay

### II.1. Materials and Instruments

| Materials | Vendor | Cat# |
|---|---|---|
| Ketanserin Hydrochloride, | PerkinElmer | NET791250UC |
| [Ethylene-3H]- | | |
| Ketanserin | MedChemExpress | HY-10562 |
| Bovine Serum Albumin (BSA) | Sigma | A1933 |
| Calcium chloride (CaCl₂) | Sigma | C5670 |
| Tris(hydroxymethyl)aminomethane | Alfa Aesar | A18494 |
| (Tris) | | |
| Polyethylenimine, branched (PEI) | Sigma | 408727 |

### II.2. Instrumentation and Consumables

| Item | Supplier | Cat# |
|---|---|---|
| Microbeta² Microplate Counter | PerkinElmer | 2450-0060 |
| UniFilter-96 GF/B | PerkinElmer | 6005177 |
| TopSeal | Biotss | SF-800 |
| MicroBeta Filtermate-96 | PerkinElmer | D961962 |
| Seven Compact pH meter | Mettler Toledo | S220 |
| Ultrapure Water Meter | Sichuan Ulupure | UPH-III-20T |
| Benchtop Centrifuge | Hunan Xiangyi | L550 |
| Microplate Shaker | Allsheng | MX100-4A |
| 384-Well Polypropylene Microplate | Labcyte | PP-0200 |
| 96 Round Well Plate | Corning | 3799 |
| 96 Round Deep Well Plate | Axygen | P-DW-11-C |
| Echo | LABCYTE | 550 |

### II.3 Experiment Procedure

Prepare the assay buffer following the table below;

| Reagent | Concentration |
|---|---|
| Tris | 50 mM |
| CaCl₂ | 4 mM |
| BSA | 0.1% (w/v) |

Adjust pH to 7.4 followed by 0.2 μM sterile filtration

ii. Preparation of 8 doses of reference and test compounds starting from 10 mM stock solution by 5-fold serial dilutions with 100%;
iii. Prepare (v/v) DMSO: a. 50 μl/well of 0.5% (v/v) PEI is added to UniFilter-96 GF/B plates. The plates are sealed and incubates at 4° C. for 3 hrs; b. After incubation, the plates are washed 3 times with ice-cold wash buffer (50 mM Tris, pH7.4);
iv. Preparation of assay plates: a. Cell membrane are diluted with assay buffer and 330 μl/well is added to 96 round deep well plates to reach a concentration of 20 μg/well; b. 8 concentrations of reference or test compounds are prepared and 110 μl/well is added to 96 round deep well plates; c. [3H]-ketanserin is diluted with assay buffer to 5 nM (5× final concentration) and 110 μl/well is added to 96 round deep well plates.
v. The plate is centrifuged at 1000 rpm for 30 secs and then agitated at 600 rpm, R.T. for 5 min.
vi. The plates are sealed and incubates at 27° C. for 90 min.
vii. The incubation is stopped by vacuum filtration onto GF/B filter plates followed by 4 times washing with ice-cold wash buffer (50 mM Tris, pH7.4).
viii. The plates are dried at 37° C. for 45 min.
ix. The filter plates are sealed and 40 μl/well of scintillation cocktail is added.
x. The plate is read by using a Microbeta2 microplate counter.

### Data Analysis:

For reference and test compounds, the results are expressed as % Inhibition, using the normalization equation: N=100-100×(U−C2)/(C1−C2), where U is the unknown value, C1 is the average of high controls, and C2 is the average of low controls. The IC50 is determined by fitting percentage of inhibition as a function of compound concentrations with Hill equation using XLfit.

### Example B2: Human, Rat and Mouse Liver Microsomes Stability Objective

The objective of this study was to estimate in vitro metabolic stability of representative compounds of the application in pooled human and male mouse liver microsomes. The concentrations of parent compounds in reaction systems were evaluated by LC-MS/MS for estimating the stability in pooled human and male mouse liver microsomes. The in vitro intrinsic clearances of test compounds were determined as well.

### Protocol

A master solution in the “Incubation Plate” containing phosphate buffer, ultra-pure H₂O, MgCl₂ solution and liver microsomes was made according to Table 3. The mixture was pre-warmed at 37° C. water bath for 5 minutes.

TABLE 3
Preparation of master solution

| Reagent | Stock Concentration | | Volume | | Final Concentration | |
|---|---|---|---|---|---|---|
| Phosphate buffer | 200 | mM | 200 | μL | 100 | mM |
| Ultra-pure H₂O | — | | 106 | μL | — | |
| MgCl₂ solution | 50 | mM | 40 | μL | 5 | mM |
| Microsomes | 20 | mg/mL | 10 | μL | 0.5 | mg/mL |

40 μL of 10 mM NADPH solution was added to each well. The final concentration of NADPH was 1 mM. The negative control samples were prepared by replacing NADPH with 40 μL of ultra-pure H₂O. Samples were prepared in duplicate. Negative controls were prepared in singlet.

The reaction was started with the addition of 4 μL of 200 μM test compounds or control compounds to each master solution to get the final concentration of 2 μM. This study was performed in duplicate.

Aliquots of 50 μL were taken from the reaction solution at 0, 15, 30, 45 and 60 minutes. The reaction solutions were stopped by the addition of 4 volumes of cold methanol with IS (100 nM alprazolam, 200 nM imipramine, 200 nM labetalol and 2 μM ketoprofen). Samples were centrifuged at 3,220 g for 40 minutes. Aliquot of 90 μL of the supernatant was mixed with 90 μL of ultra-pure H₂O and then was used for LC-MS/MS analysis.

LC/MS analysis was performed for all samples from this study using a Shimadzu liquid chromatograph separation system equipped with degasser DGU-20A_{5R}; solvent delivery unit LC-30AD; system controller SIL-30AC; column oven CTO-30A; CTC Analytics HTC PAL System. Mass spectrometric analysis was performed using a Triple Quad™ 5500 instrument.

All calculations were carried out using Microsoft Excel. Peak area ratios of test compound to internal standard (listed in the below table) were determined from extracted ion chromatograms.

All calculations were carried out using Microsoft Excel. Peak areas were determined from extracted ion chromatograms. The slope value, k, was determined by linear regression of the natural logarithm of the remaining percentage of the parent drug vs. incubation time curve.

The in vitro half-life (in vitro t_{1/2}) was determined from the slope value:

$$in vitro {t}_{1 / 2} = - ( 0.693 / k )$$

Conversion of the in vitro t_{1/2} (min) into the in vitro intrinsic clearance (in vitro CLᵢₙₜ, in μL/min/mg proteins) was done using the following equation (mean of duplicate determinations):

$$in vitro {CL}_{int} = ( \frac{0.693}{( {t}_{1 / 2} )} ) * ( \frac{volume of incubation ( μ L )}{amount of proteins ( mg )} )$$

For the compound or control compound that showed an initial fast disappearance followed by a slow disappearance, only the time points that were within the initial rate were included in the calculation.

### Results & Discussion

Human, rat and mouse liver microsomes contain a wide variety of drug metabolizing enzymes and are commonly used to support in vitro ADME (absorption, distribution, metabolism and excretion) studies. These microsomes were used to examine the potential first-pass metabolism by-products of orally administered drugs. Representative compounds of the application were evaluated for their stability in human, rat and mouse liver microsomes. Table 4 and Table 5 shows the results of the stability studies.

TABLE 4

| | | | | | Predicted |
|---|---|---|---|---|---|
| | | | | Scaled-up | hepatic |
| | | | CLᵢₙₜ | CLᵢₙₜ | CL |
| Compound | | T_{1/2} | (μL/min/mg | (mL/min/ | (mL/min/ |
| I.D. | Species | (min) | protein) | Kg) | kg) |
| Diclofenac | Human | 10.35 | 133.94 | 167.98 | 18.43 |
| | Rat | 14.62 | 94.80 | 169.89 | 41.66 |
| | Mouse | 43.85 | 31.61 | 138.28 | 54.52 |
| II-1 | Human | 39.04 | 35.50 | 44.53 | 14.13 |
| (Example 1) | Rat | 14.77 | 93.82 | 168.12 | 41.56 |
| | Mouse | 16.46 | 84.19 | 368.33 | 72.33 |
| II-2 | Human | 107.20 | 12.93 | 16.22 | 9.09 |
| (Example 3) | Rat | 18.28 | 75.81 | 135.86 | 39.25 |
| | Mouse | 36.72 | 37.74 | 165.12 | 58.25 |
| I-2 | Human | 921.04 | 1.50 | 1.89 | 1.73 |
| | Rat | 12.57 | 110.26 | 197.58 | 43.15 |
| | Mouse | 37.19 | 37.27 | 163.05 | 57.99 |
| I-11 | Human | 53.38 | 25.97 | 32.57 | 12.66 |
| | Rat | 15.29 | 90.65 | 162.44 | 41.20 |
| | Mouse | 18.17 | 76.29 | 333.77 | 70.89 |

TABLE 5

| | | | Remaining Percentage (%) | | |
|---|---|---|---|---|---|
| Compound ID | Species | Assay Format | 0 min | 30 min | 60 min |
| Diclofenac | Human | With NADPH | 100.00 | 4.80 | 1.80 |
| | | Without NADPH | 100.00 | 91.86 | 91.86 |
| | Rat | With NADPH | 100.00 | 10.72 | 5.82 |
| | | Without NADPH | 100.00 | 100.00 | 96.90 |
| | Mouse | With NADPH | 100.00 | 51.50 | 38.74 |
| | | Without NADPH | 100.00 | 105.81 | 102.49 |
| II-1 | Human | With NADPH | 100.00 | 65.37 | 34.47 |
| (Example 1) | | Without NADPH | 100.00 | 89.91 | 83.49 |
| | Rat | With NADPH | 100.00 | 20.42 | 5.99 |
| | | Without NADPH | 100.00 | 86.92 | 87.15 |
| | Mouse | With NADPH | 100.00 | 31.98 | 8.00 |
| | | Without NADPH | 100.00 | 117.83 | 113.85 |
| II-2 | Human | With NADPH | 100.00 | 87.48 | 67.85 |
| (Example 3) | | Without NADPH | 100.00 | 100.69 | 86.47 |
| | Rat | With NADPH | 100.00 | 26.32 | 10.29 |
| | | Without NADPH | 100.00 | 92.41 | 83.54 |
| | Mouse | With NADPH | 100.00 | 57.37 | 32.23 |
| | | Without NADPH | 100.00 | 99.08 | 86.24 |
| I-2 | Human | With NADPH | 100.00 | 111.62 | 95.59 |
| | | Without NADPH | 100.00 | 115.22 | 108.70 |
| | Rat | With NADPH | 100.00 | 14.82 | 3.66 |
| | | Without NADPH | 100.00 | 106.67 | 104.17 |
| | Mouse | With NADPH | 100.00 | 56.32 | 32.69 |
| | | Without NADPH | 100.00 | 108.70 | 97.83 |
| I-11 | Human | With NADPH | 100.00 | 72.72 | 45.89 |
| | | Without NADPH | 100.00 | 97.58 | 92.20 |
| | Rat | With NADPH | 100.00 | 22.43 | 6.59 |
| | | Without NADPH | 100.00 | 102.53 | 85.67 |
| | Mouse | With NADPH | 100.00 | 31.50 | 10.14 |
| | | Without NADPH | 100.00 | 94.39 | 80.37 |

The data in Tables 4 and 5 show that the compounds of the application exhibit a spectrum of stability profiles in liver microsomes across different species, including human, rat and mouse.

### Example B3: Psychedelic-Like Effect of Compounds of Formula I

The effect of different doses of representative compounds of Formula I were evaluated on head-twitch response (HTR) and other behavioural responses as behavior-based models of psychedelic activity.

### Protocols

### Mouse Head Twitch

Male, C57BL/6J mice (body weight range 20-30 g) were dosed with the appropriate dose of test article, and following a 1-minute pre-treatment time, placed in individual observation chambers. Animals were visually assessed for the incidence head twitches continuously over a 1 hr period. Head twitches were defined as a rapid jerk of the head which was not elicited by an external tactile stimulus (Come and Pickering, Psychopharmacologia, 1967, 11(1): 65-78). Each head twitch was individually counted by a trained observer, and the data was expressed as the mean±SEM of 6-10 mice per group. Mice were used in a single experiment only.

### Results and Discussion

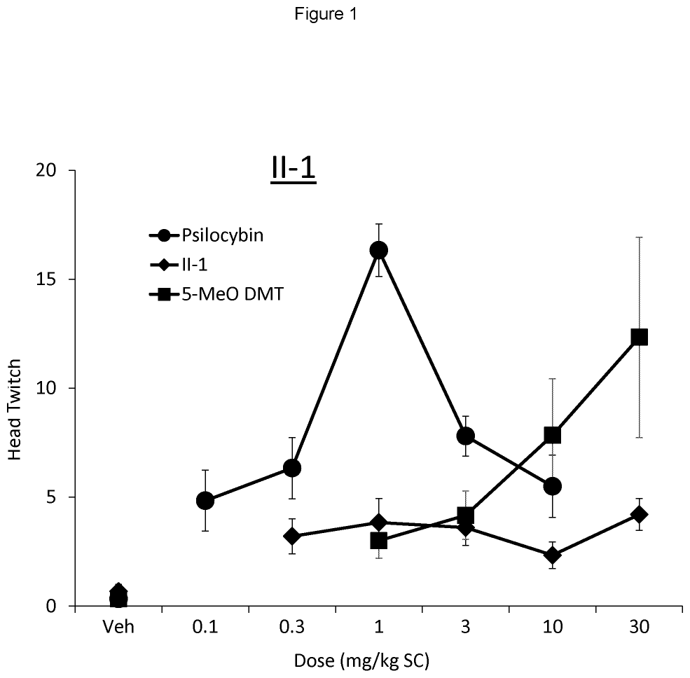

The head twitch response of exemplary compound II-1 (1-30 mg/kg SC) was compared to 5-MeO DMT and psilocybin (FIG. 1, Table 6).

TABLE 6

| | Psilocybin | II-1 | 5-MeO DMT |
|---|---|---|---|
| Vehicle (Veh) | 0.3 | 0.7 | 0.3 |
| 0.1 | 4.8 | | |
| 0.3 | 6.3 | 3.2 | |
| 1 | 16.3 | 3.8 | 3.0 |
| 3 | 7.8 | 3.6 | 4.2 |
| 10 | 5.5 | 2.3 | 7.8 |
| 30 | | 4.2 | 12.3 |
| Veh | 0.3 | 0.3 | |
| 0.1 | 1.4 | | |
| 0.3 | 1.4 | 0.8 | |
| 1 | 1.2 | 1.1 | 0.8 |
| 3 | 0.9 | 0.8 | 1.1 |
| 10 | 1.4 | 0.6 | 2.6 |
| 30 | | 0.7 | 4.6 |

The data in Table 6 shows that psilocybin (0.1-10 mg/kg SC) produced a dose related increase in the incidence of head twitch response in male C57BL/6 mice, which reached a peak at the 1 mg/kg dose. At 3 and 10 mg/kg SC doses, the incidence of head twitches following psilocybin declined, resulting in an inverted U-shaped dose response. The decline in head twitches at these higher doses was accompanied by decreased locomotion and decreased core body temperature.

5-MeO DMT (1-30 mg/kg SC) produced a monotonic increase in head twitches, although over the 3-30 mg/kg doses other behavioural signs associated with 5-HT syndrome (forepaw treading, straub tail, body twitches, backwards walking) emerged and increased in incidence across these doses. Consequently head twitches in the 30 mg/kg became somewhat variable due to response competition and marked 5-HT syndrome signs compromising the expression of head twitches in some test subjects.

In contrast with psilocybin and 5-MeO DMT, exemplary compound II-1 (0.3-30 mg/kg SC) elicited only a modest head twitch response with a peak response of 4.2±0.7 twitches at 30 mg/kg although the lower doses were also associated with incidence of head twitches. No overt signs of hypolocomotion or 5-HT syndrome was recorded over this dose range, except for 2/5 (40%) mice pretreated with 30 mg/kg dose, where some 5-HT syndrome associated behaviours were noted. Therefore the low expression of head twitches, at least over the 0.3-10 mg/kg dose range, did not appear to be a consequence of alternative, competing behaviours.

### Rat Behavioural Test

Male, Sprague-Dawley rats (body weight range 250-400 g) are dosed with the appropriate dose of test article and following a 1-minute pre-treatment time, placed in locomotor activity boxes (dimensions 17″ W×17″ L×12″ H) and continuously monitored for a 1 hr period with data collected into 10 minute time bins. Animals are visually assessed for overt behavioural signs, including behaviours characteristic of 5-HT_{2A} receptor activation (wet dog shakes, back muscle contractions), 5-HT_{2A} receptor activation (yawning, penile grooming) and 5-HT_{1A} behaviours (forepaw treading, hindlimb abduction) (Halberzettl et al, Behav Brain Res. 256: 328-345, 2013). Additional behavioural and somatic signs characteristic of 5-HT syndrome (e.g. tremor, salivation, flat body posture, core body temperature change) are also measured. Simultaneously, the spontaneous activity of the rats is measured using an automated tracking system (Med Associates, VT, USA). Activity data collected included total distance traveled, rearing counts and ambulatory episodes. All data are expressed as the mean+SEM of 6-10 rats per group.

### Drug Discrimination in the Rat

Male Sprague-Dawley rats are initially food restricted by presentation of 18-20 g food at day end (single housing). After 7 days acclimatisation to the food restriction procedure, they are trained daily to lever press for food (45 mg Bioserve pellet) in standard 2-lever operant conditioning chambers controlled by Med-PC software over a period of 1 week (Med. Associates Ins., St. Albans, VT). The rats are trained to lever press for food to an FR10 value (i.e. 10 lever presses for a single food reward). Once stable food responding is acquired to both response levers, discrimination training began. Over a period of 20-50 training sessions, the rats are trained to associate one lever to a psilocybin training dose of 1 mg/kg SC, and the second lever to a neutral stimulus (saline, SC) (Winter et al, Pharmacol Biochem Behav. 87(4): 472-480, 2007). Training sessions lasted 30-min or until the delivery of 50 pellets and continue until the animals attain appropriate stimulus control (defined as six consecutive sessions where animals makes no more than 16 lever presses before the delivery of the first reward, and at least 95% total responses on the appropriate lever). The rats continue to receive daily food ration in their home cage at day end.

Once trained, tests of substitution are conducted. On test days, both levers are designated active, i.e., every 10th response on either lever results in delivery of a food pellet. Test sessions continue until 50 pellets have been obtained or 30 min has elapsed. During these sessions response rate is also measured.

While the present application has been described with reference to examples, it is to be understood that the scope of the claims should not be limited by the embodiments set forth in the examples, but should be given the broadest interpretation consistent with the description as a whole.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the application described and claimed herein.

1. A compound of Formula I: 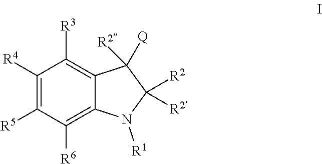
or a pharmaceutically acceptable salt, solvate, and/or prodrug thereof,
wherein:
R¹ is selected from C(O)R⁷, CO₂R⁷, C(O)N(R⁷)(R^{7′}), S(O)R⁷, SO₂R⁷, C₁₋₆alkyleneR⁷, and R⁷;
Q is Q3: 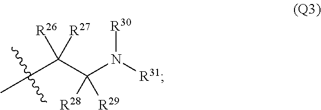 [symbol]
over a bond means that the bond is attached to a remaining portion of the compound;
R², R^{2′}, and R^{2″} are independently selected from H, halo, C₁₋₆alkyl, C₁₋₆alkoxy, C₁₋₆alkyleneN(R^{2a})(R^{2b}), and SC₁₋₆alkyl;
R^{2a} and R^{2b} are independently selected from H and C₁₋₆alkyl;
R³, R⁴, R⁵, and R⁶ are independently selected from H, halo, N(R^{5′})(R^{6′}), C₁₋₆alkyl, C₁₋₆alkoxy, C₁₋₆ alkyleneN(R^{5′})(R^{6′}), and SC₁₋₆alkyl, the latter four groups being optionally substituted with one or two substituents independently selected from OH and C₁₋₆alkoxy, or
two adjacent R³, R⁴, R⁵, and R⁶ are linked together to form O—(CH₂)₁₋₂O and the remaining of R³, R⁴, R⁵, and R⁶ are independently selected from H, halo, C₁₋₆alkyl, C₁₋₆alkoxy, N(R^{5′})(R^{6′}), C₁₋₆alkyleneN(R^{5′})(R⁶′), and SC₁₋₆alkyl, or
one of R³, R⁴, R⁵, and R⁶ is selected from A, O-A, and C₁₋₄alkyleneA and the remaining of R³, R⁴, R⁵, and R⁶ are independently H or halo;
R^{5′} and R^{6′} are independently selected from H and C₁₋₆alkyl;
A is selected from phenyl, C₃₋₆cycloalkyl, 3- to 6-membered heterocycloalkyl comprising 1 to 4 heteromoieties independently selected from O, S, S(O), SO₂, N, and NR⁵⁴ and 5- to 6-membered heteroaryl comprising 1 to 4 heteromoieties independently selected from O, S, S(O), SO₂, N, and NR⁵⁴, wherein the phenyl, C₃₋₁₀cycloalkyl, 3- to 6-membered heterocycloalkyl, and 5- to 6-membered heteroaryl are optionally substituted with one or more substituents independently selected from halo, C₁₋₄alkyl, OC₁₋₄alkyl, N(R^{54a})(R^{54b}), C₁₋₄alkyleneN(R^{54a})(R^{54b}), and SC₁₋₄alkyl;
R⁷ is selected from H, C₁₋₆alkyl, C₂₋₆alkenyl, C₂₋₆alkynyl, phenyl, C₃₋₆cycloalkyl, 3- to 6-membered heterocycloalkyl comprising 1 to 4 heteromoieties independently selected from O, S, N, and NR^{7″}, and 5- to 6-membered heteroaryl comprising 1 to 4 heteromoieties independently selected from O, S, N, and NR^{7″}, wherein the latter 7 groups are optionally substituted with one or more substituents independently selected from halo, OR⁵⁵, N(R⁵⁵)(R⁵⁶), and SR⁵⁵ and/or the C₁₋₆alkyl is optionally interrupted by one to three heteromoieties independently selected from O, C(O), CO₂, and NR⁵⁷;
R^{7′} is selected from H and C₁₋₆alkyl;
R^{7″} is selected from H and C₁₋₆alkyl;
R²⁶, R²⁷, R²⁸, and R²⁹ are independently selected from H, halo, and C₁₋₆alkyl;
R³⁰ and R³¹ are independently selected from H, C₁₋₆alkyl, and C(O)C₁₋₆alkyl, or
R³⁰ and R³¹, together with the N atom to which they are bound, form a 3- to 6-membered heterocyclic ring which optionally comprises one or two additional heteromoieties independently selected from O, S, S(O), SO₂, N, and NR⁵⁸;
R⁵⁴, R⁵⁵, R⁵⁵, R⁵⁷, and R⁵⁸ are independently selected from H and C₁₋₆alkyl;
R^{54a} and R^{54b} are independently selected from H and C₁₋₆alkyl; and
available hydrogen atoms are optionally and independently substituted with a fluorine atom or a chlorine atom and/or available atoms are optionally substituted with alternate isotope thereof,
provided that when R²⁶, R¹⁷, R²⁸, and R²⁹ are all H and R³⁰ and R³¹ are H or CH₃, and:
R¹, R², R^{2′}, R^{2″}, R³, R⁴, R⁵, and R⁶ are all H,
R¹, R², R^{2′}, R^{2″}, R³, R⁵, and R⁶ are all H and R⁴ is OCH₃, or
R¹, R², R^{2′}, R^{2″}, R³, R⁴, R⁵, and R⁶ are all H and R⁵ is OCH₃,
then the compound of Formula I is an (R)- or (S)-enantiomer of the carbon to which Q is attached.

2. The compound of claim 1, wherein R²⁶, R²⁷, R²⁸, and R²⁹ are independently selected from H and D.

3. The compound of claim 1, wherein Q is selected from one of the following groups:

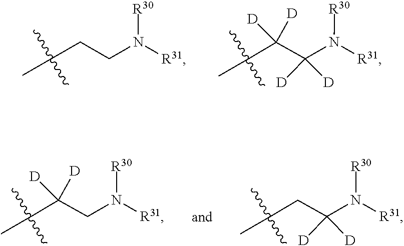

4. The compound of claim 3, wherein R³⁰ and R³¹ are independently selected from H, C₁₋₆alkyl, and C(O)C₁₋₆alkyl, wherein available hydrogen atoms are optionally and independently substituted with a fluorine atom or a deuterium atom.

5. The compound of claim 4, wherein R³⁰ and R³¹ are independently selected from H, D, C₁₋₆alkyl, C₁₋₆fluoroalkyl, C₁₋₆deuteroalkyl, C₁₋₆alkoxy, C(O)C₁₋₆fluoroalkyl, and C(O)C₁₋₆deuteroalkyl.

6. The compound of claim 5, wherein R³⁰ and R³¹ are independently selected from H, D, CH₃, CF₃, and CH(CH₃)₂.

7. (canceled)

8. (canceled)

9. The compound of claim 1, wherein R¹ is selected from C(O)R⁷, CO₂R⁷, C(O)N(R⁷)(R^{7′}), S(O)R⁷, SO₂R⁷, C₁₋₄alkyleneR⁷, and R⁷, wherein R⁷ is selected from H, C₁₋₄alkyl, C₂₋₆alkenyl, C₂₋₆alkynyl, phenyl, C₃₋₆cycloalkyl, 3- to 6-membered heterocycloalkyl comprising 1 to 4 heteromoieties independently selected from O, S, N, and NR^{7″}, and 5- to 6-membered heteroaryl comprising 1 to 4 heteromoieties independently selected from O, S, N, and NR^{7″}, wherein the latter 7 groups are optionally substituted with one or more substituents independently selected from halo, OR⁵⁵, N(R⁵⁵)(R⁵⁶), and SR⁵⁵ and/or the C₁₋₄alkyl is optionally interrupted by one to three heteromoieties independently selected from O, C(O), CO₂, and NR⁵⁷; and wherein available hydrogen atoms are optionally and independently substituted with a fluorine atom or a deuterium atom.

10. (canceled)

11. (canceled)

12. (canceled)

13. (canceled)

14. (canceled)

15. (canceled)

16. The compound of claim 1, wherein R⁷ is selected from H, D, C₁₋₄alkyl, C₁₋₄fluoroalkyl, and C₁₋₄deuteroalkyl.

17. (canceled)

18. (canceled)

19. The compound of claim 1, wherein R², R^{2′}, and R^{2″} are independently selected from H, halo, C₁₋₄alkyl, C₁₋₄alkoxy, C₁₋₄fluoroalkoxy, C₁₋₃alkyleneN(R^{2a})(R^{2b}), and SC₁₋₄alkyl wherein available hydrogen atoms are optionally and independently substituted with a fluorine atom or a deuterium atom.

20. The compound of claim 19, wherein R², R^{2′}, and R^{2″} are independently selected from H, D, F, Cl, C₁₋₄alkyl, C₁₋₄fluoroalkyl, C₁₋₄deuteroalkyl, C₁₋₄alkoxy, C₁₋₄fluoroalkoxy, C₁₋₄deuteroalkoxy, C₁₋₃alkyleneN(R^{2a})(R^{2b}), SC₁₋₄alkyl, SC₁₋₄fluoroalkyl, and SC₁₋₄deuteroalkyl.

21. (canceled)

22. The compound of claim 20, wherein R², R^{2′}, and R^{2″} are all H or R², R^{2′}, and R^{2″} are all D.

23. (canceled)

24. The compound of claim 1, wherein R³, R⁴, R⁵, and R⁶ are independently selected from H, halo, C₁₋₄alkyl, C₁₋₄alkoxy, C₁₋₄alkyleneN(R^{5′})(R^{6′}), and SC₁₋₄alkyl, the latter four groups being optionally substituted with one or two substituents independently selected from OH and C₁₋₄alkoxy, wherein available hydrogen atoms are optionally and independently substituted with a fluorine atom or a deuterium atom.

25. The compound of claim 24, wherein R³, R⁴, R⁵, and R⁶ are independently selected from H, D, F, Cl, Br, N(R^{5′})(R^{6′}), C₁₋₄alkyl, C₁₋₄fluoroalkyl, C₁₋₄deuteroalkyl, C₁₋₄alkoxy, C₁₋₄fluoroalkoxy, C₁₋₄deuteroalkoxy, C₁₋₄alkyleneN(R^{5′})(R⁶′), SC₁₋₄alkyl, SC₁₋₄fluoroalkyl, and SC₁₋₄deuteroalkyl, the latter ten groups being optionally substituted with one or two substituents independently selected from OH, C₁₋₄alkoxy, C₁₋₄fluoroalkoxy, and C₁₋₄deuteroalkoxy.

26. (canceled)

27. (canceled)

28. The compound of claim 25, wherein one of R³, R⁴, R⁵, and R⁶ is selected from C₁₋₃alkoxy, C₁₋₃fluoroalkoxy, and C₁₋₃deuteroalkoxy substituted with one or two substituents selected from OH, C₁₋₃alkoxy, C₁₋₃fluoroalkoxy, and C₁₋₃deuteroalkoxy, and the remaining of R³, R⁴, R⁵, and R⁶ are independently selected from H, D, CH₃O, CD₃O, and CF₃O.

29. (canceled)

30. The compound of claim 28, wherein one to three of R³, R⁴, R⁵, and R⁶ are independently selected from H, D, F, CH₃, CD₂H, CDH₂, CD₃, CF₃, CHF₂, CH₂CH₃, CH(CH₃)₂, CH₂CH₂D, CH₂CD₂H, CD₂CD₃, CH₃O, CD₂HO, CDH₂O, CD₃O, CF₃O, CHF₂O, CH₃CH₂O, CH(CH₃)₂O, CH₂DCH₂O, CD₂HCH₂O, and CD₃CD₂O and the remaining of R³, R⁴, R⁵, and R⁶ are selected from H and D.

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. The compound of claim 1, wherein the compound of Formula I has one of the following structures: 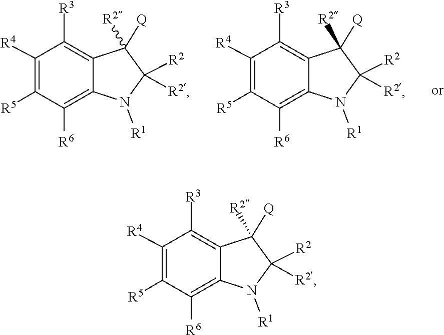
or a mixture thereof.

58. (canceled)

59. The compound of claim 1 selected from:

| Compound I.D. | Structure |
|---|---|
| (R)-I-1 | 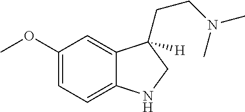 |
| (S)-I-1 | 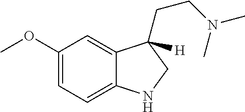 |
| I-2 | 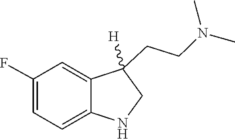 |
| I-3 | 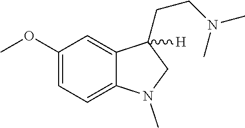 |
| (S)-I-3 | 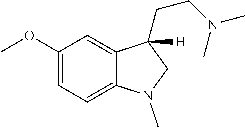 |
| (R)-I-3 | 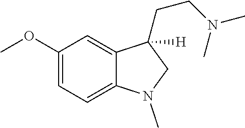 |
| (S)-I-4 | 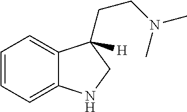 |
| (R)-I-4 | 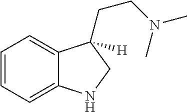 |
| I-6 | 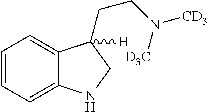 [SMILES: [2H]C([2H])([2H])N(CCC1CNc2ccccc21)C([2H])([2H])[2H]] |
| I-7 | 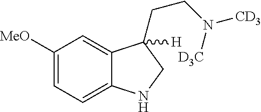 [SMILES: [2H]C([2H])([2H])N(CCC1CNc2ccc(OC)cc21)C([2H])([2H])[2H]] |
| I-9 | 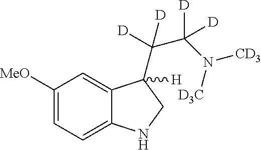 [SMILES: [2H]C([2H])([2H])N(C([2H])([2H])[2H])C([2H])([2H])C([2H])([2H])C1CNc2ccc(OC)cc21] |
| I-10 | 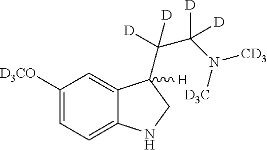 |
| I-11 | 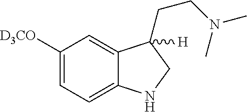 |
| I-12 | 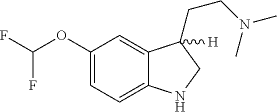 [SMILES: CN(C)CCC1CNc2ccc(OC(F)F)cc21] |
| I-13 | 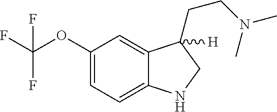 [SMILES: CN(C)CCC1CNc2ccc(OC(F)(F)F)cc21] |
| I-14 | 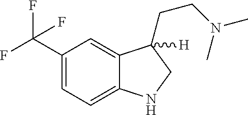 [SMILES: CN(C)CCC1CNc2ccc(C(F)(F)F)cc21] |
| I-15 | 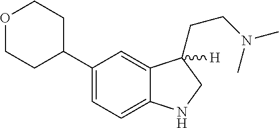 [SMILES: CN(C)CCC1CNc2ccc(C3CCOCC3)cc21] |
| I-16 | 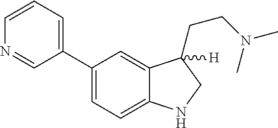 [SMILES: CN(C)CCC1CNc2ccc(-c3cccnc3)cc21] |
| I-17 | 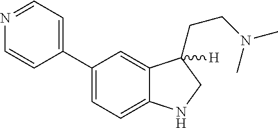 [SMILES: CN(C)CCC1CNc2ccc(-c3ccncc3)cc21] |
| I-18 | 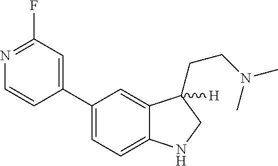 [SMILES: CN(C)CCC1CNc2ccc(-c3ccnc(F)c3)cc21] |
| I-19 | 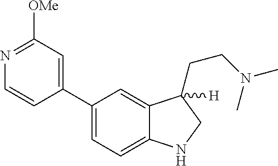 [SMILES: COc1cc(-c2ccc3c(c2)C(CCN(C)C)CN3)ccn1] |
| I-20 | 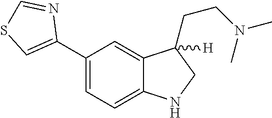 [SMILES: CN(C)CCC1CNc2ccc(-c3cscn3)cc21] |
| I-21 | 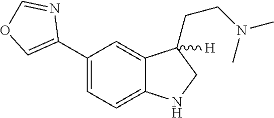 [SMILES: CN(C)CCC1CNc2ccc(-c3cocn3)cc21] |
| I-22 | 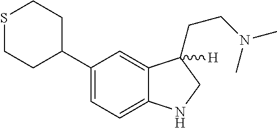 [SMILES: CN(C)CCC1CNc2ccc(C3CCSCC3)cc21] |
| I-23 | 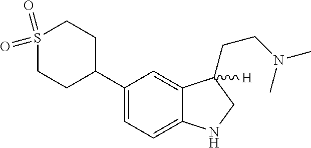 [SMILES: CN(C)CCC1CNc2ccc(C3CCS(=O)(=O)CC3)cc21] |
| I-24 | 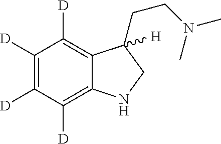 [SMILES: [2H]c1c([2H])c([2H])c2c(c1[2H])NCC2CCN(C)C] |
| I-25 | 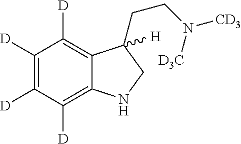 [SMILES: [2H]c1c([2H])c([2H])c2c(c1[2H])NCC2CCN(C([2H])([2H])[2H])C([2H])([2H])[2H]] |
| I-26 | 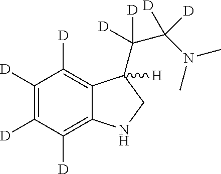 [SMILES: [2H]c1c([2H])c([2H])c2c(c1[2H])NCC2C([2H])([2H])C([2H])([2H])N(C)C] |
| I-27 | 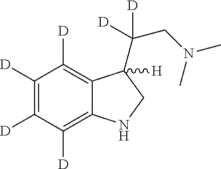 [SMILES: [2H]c1c([2H])c([2H])c2c(c1[2H])NCC2C([2H])([2H])CN(C)C] |
| I-28 | 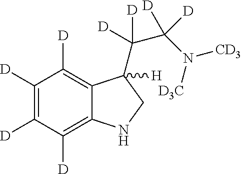 [SMILES: [2H]c1c([2H])c([2H])c2c(c1[2H])NCC2C([2H])([2H])C([2H])([2H])N(C([2H])([2H])[2H])C([2H])([2H])[2H]] |
| I-30 | 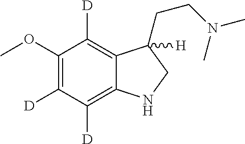 |
| I-31 | 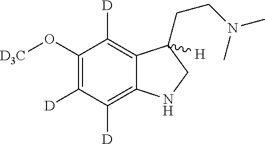 [SMILES: [2H]c1c([2H])c(OC([2H])([2H])[2H])c([2H])c2c1NCC2CCN(C)C] |
| I-32 | 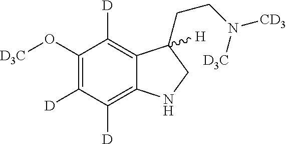 [SMILES: [2H]c1c([2H])c(OC([2H])([2H])[2H])c([2H])c2c1NCC2CCN(C([2H])([2H])[2H])C([2H])([2H])[2H]] |
| I-33 | 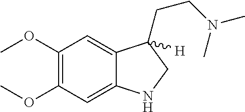 |
| (S)-I-33 | 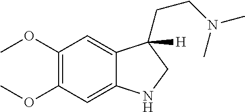 |
| (R)-I-33 | 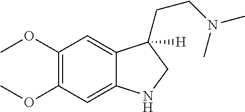 |
| I-36 | 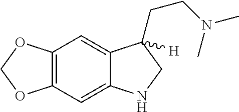 [SMILES: CN(C)CCC1CNc2cc3c(cc21)OCO3] |
| (S)-I-36 | 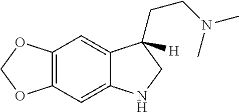 [SMILES: CN(C)CC[C@@H]1CNc2cc3c(cc21)OCO3] |
| (R)-I-36 | 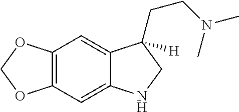 [SMILES: CN(C)CC[C@H]1CNc2cc3c(cc21)OCO3] |
| I-39 | 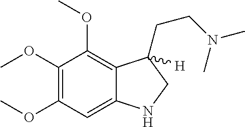 |
| I-40 | 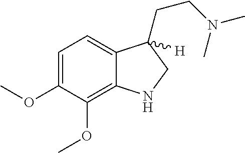 |
| I-41 | 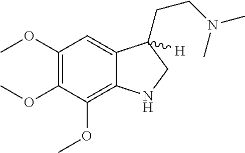 |
| I-43 | 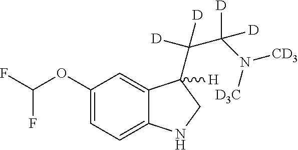 [SMILES: [2H]C([2H])([2H])N(C([2H])([2H])[2H])C([2H])([2H])C([2H])([2H])C1CNc2ccc(OC(F)F)cc21] |
| I-44 | 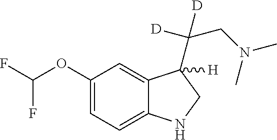 [SMILES: [2H]C([2H])(CN(C)C)C1CNc2ccc(OC(F)F)cc21] |
| I-45 | 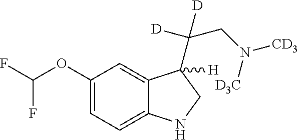 [SMILES: [2H]C([2H])(CN(C([2H])([2H])[2H])C([2H])([2H])[2H])C1CNc2ccc(OC(F)F)cc21] |
| I-107 | 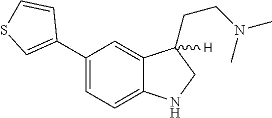 [SMILES: CN(C)CCC1CNc2ccc(-c3ccsc3)cc21] |
| (S)-I-107 | 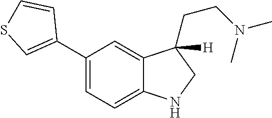 [SMILES: CN(C)CC[C@@H]1CNc2ccc(-c3ccsc3)cc21] |
| (R)-I-107 | 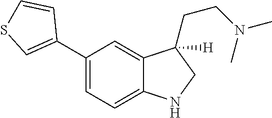 [SMILES: CN(C)CC[C@H]1CNc2ccc(-c3ccsc3)cc21] |
| (S)-I-108 | 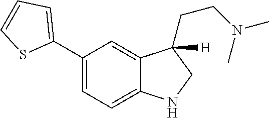 [SMILES: CN(C)CC[C@@H]1CNc2ccc(-c3cccs3)cc21] |
| I-109 | 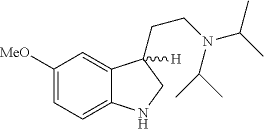 [SMILES: COc1ccc2c(c1)C(CCN(C(C)C)C(C)C)CN2] |
| I-110 | 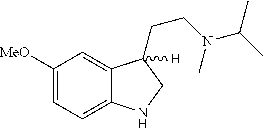 [SMILES: COc1ccc2c(c1)C(CCN(C)C(C)C)CN2] |
| I-111 | 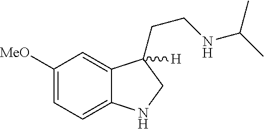 [SMILES: COc1ccc2c(c1)C(CCNC(C)C)CN2] |
| I-112 | 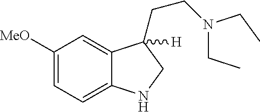 [SMILES: CCN(CC)CCC1CNc2ccc(OC)cc21] |
| I-113 | 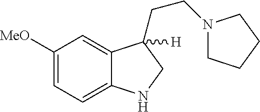 [SMILES: COc1ccc2c(c1)C(CCN1CCCC1)CN2] |
| I-114 | 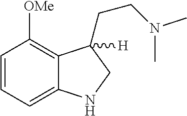 [SMILES: COc1cccc2c1C(CCN(C)C)CN2] |
| (S)-I-114 | 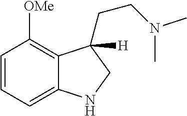 [SMILES: COc1cccc2c1[C@H](CCN(C)C)CN2] |
| (R)-I-114 | 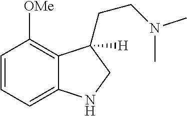 [SMILES: COc1cccc2c1[C@@H](CCN(C)C)CN2] |
| I-115 | 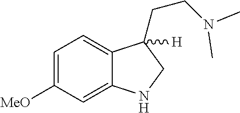 [SMILES: COc1ccc2c(c1)NCC2CCN(C)C] |
| (S)-I-115 | 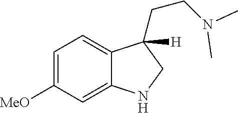 [SMILES: COc1ccc2c(c1)NC[C@H]2CCN(C)C] |
| (R)-I-115 | 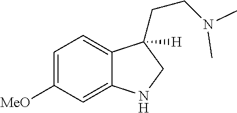 [SMILES: COc1ccc2c(c1)NC[C@@H]2CCN(C)C] |
| I-116 | 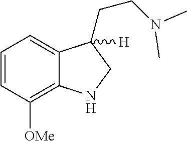 [SMILES: COc1cccc2c1NCC2CCN(C)C] |
| (S)-I-116 | 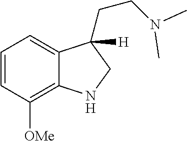 [SMILES: COc1cccc2c1NC[C@H]2CCN(C)C] |
| (R)-I-116 | 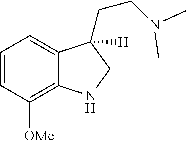 [SMILES: COc1cccc2c1NC[C@@H]2CCN(C)C] |
| I-117 | 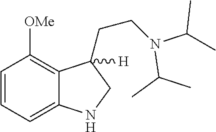 [SMILES: COc1cccc2c1C(CCN(C(C)C)C(C)C)CN2] |
| (S)-I-117 | 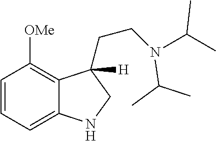 [SMILES: COc1cccc2c1[C@H](CCN(C(C)C)C(C)C)CN2] |
| (R)-I-117 | 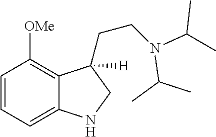 [SMILES: COc1cccc2c1[C@@H](CCN(C(C)C)C(C)C)CN2] |
| I-118 | 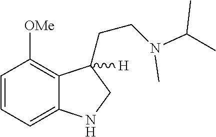 [SMILES: COc1cccc2c1C(CCN(C)C(C)C)CN2] |
| (S)-I-118 | 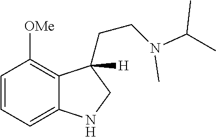 [SMILES: COc1cccc2c1[C@H](CCN(C)C(C)C)CN2] |
| (R)-I-118 | 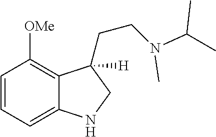 [SMILES: COc1cccc2c1[C@@H](CCN(C)C(C)C)CN2] |
| I-119 | 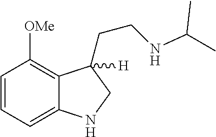 [SMILES: COc1cccc2c1C(CCNC(C)C)CN2] |
| (S)-I-119 | 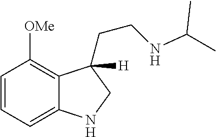 [SMILES: COc1cccc2c1[C@H](CCNC(C)C)CN2] |
| (R)-I-119 | 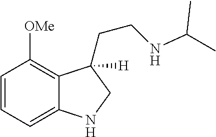 [SMILES: COc1cccc2c1[C@@H](CCNC(C)C)CN2] |
| I-120 | 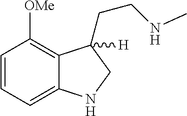 |
| (S)-I-120 | 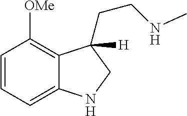 |
| (R)-I-120 | 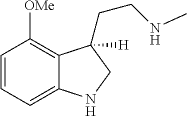 |
| I-121 | 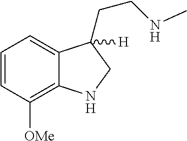 |
| (S)-I-121 | 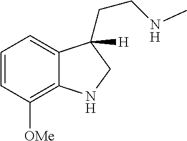 |
| (R)-I-121 | 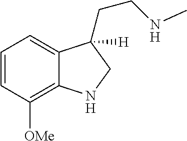 |
| I-122 | 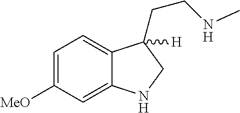 |
| (S)-I-122 | 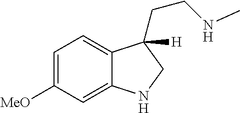 |
| (R)-I-122 | 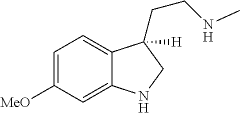 |
| I-123 | 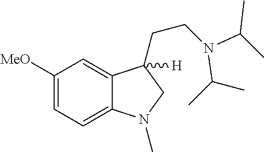 [SMILES: COc1ccc2c(c1)C(CCN(C(C)C)C(C)C)CN2C] |
| (S)-I-123 | 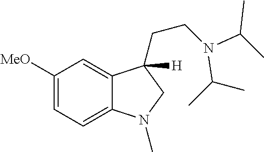 [SMILES: COc1ccc2c(c1)[C@H](CCN(C(C)C)C(C)C)CN2C] |
| (R)-I-123 | 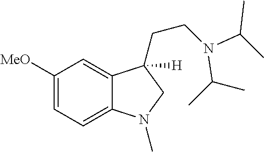 [SMILES: COc1ccc2c(c1)[C@@H](CCN(C(C)C)C(C)C)CN2C] |
| I-124 | 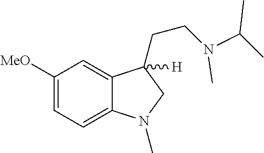 [SMILES: COc1ccc2c(c1)C(CCN(C)C(C)C)CN2C] |
| (S)-I-124 | 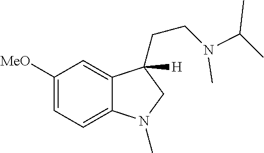 [SMILES: COc1ccc2c(c1)[C@H](CCN(C)C(C)C)CN2C] |
| (R)-I-124 | 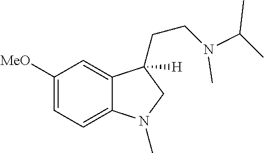 [SMILES: COc1ccc2c(c1)[C@@H](CCN(C)C(C)C)CN2C] |
| I-125 | 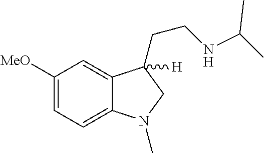 [SMILES: COc1ccc2c(c1)C(CCNC(C)C)CN2C] |
| (S)-I-125 | 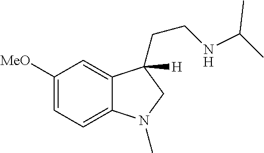 [SMILES: COc1ccc2c(c1)[C@H](CCNC(C)C)CN2C] |
| (R)-I-125 | 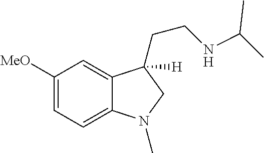 [SMILES: COc1ccc2c(c1)[C@@H](CCNC(C)C)CN2C] |
| I-126 | 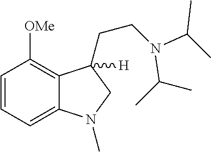 [SMILES: COc1cccc2c1C(CCN(C(C)C)C(C)C)CN2C] |
| (R)-I-126 | 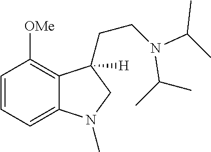 [SMILES: COc1cccc2c1[C@@H](CCN(C(C)C)C(C)C)CN2C] |
| (S)-I-126 | 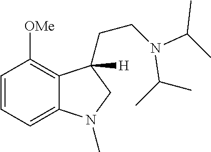 [SMILES: COc1cccc2c1[C@H](CCN(C(C)C)C(C)C)CN2C] |
| I-127 | 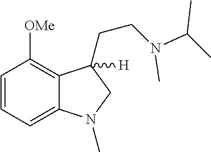 [SMILES: COc1cccc2c1C(CCN(C)C(C)C)CN2C] |
| (R)-I-127 | 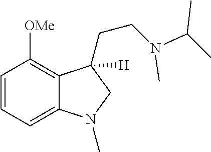 [SMILES: COc1cccc2c1[C@@H](CCN(C)C(C)C)CN2C] |
| (S)-I-127 | 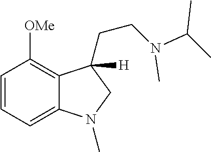 [SMILES: COc1cccc2c1[C@H](CCN(C)C(C)C)CN2C] |
| I-128 | 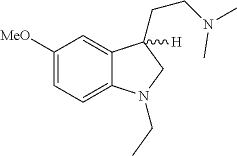 [SMILES: CCN1CC(CCN(C)C)c2cc(OC)ccc21] |
| (S)-I-128 | 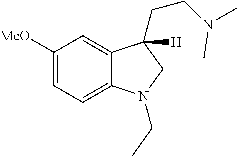 [SMILES: CCN1C[C@@H](CCN(C)C)c2cc(OC)ccc21] |
| (R)-I-128 | 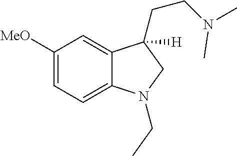 [SMILES: CCN1C[C@H](CCN(C)C)c2cc(OC)ccc21] |
| I-129 | 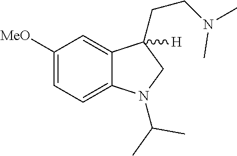 [SMILES: COc1ccc2c(c1)C(CCN(C)C)CN2C(C)C] |
| (S)-I-129 | 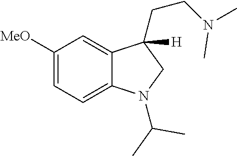 [SMILES: COc1ccc2c(c1)[C@H](CCN(C)C)CN2C(C)C] |
| (R)-I-129 | 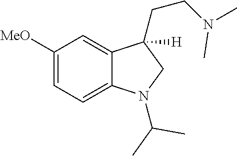 [SMILES: COc1ccc2c(c1)[C@@H](CCN(C)C)CN2C(C)C] |
| I-130 | 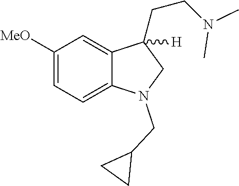 [SMILES: COc1ccc2c(c1)C(CCN(C)C)CN2CC1CC1] |
| I-131 | 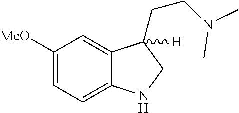 [SMILES: COc1ccc2c(c1)C(CCN(C)C)CN2] |
| I-132 | 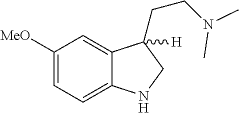 [SMILES: COc1ccc2c(c1)C(CCN(C)C)CN2] |
| I-133 | 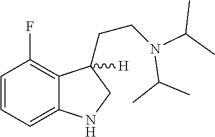 [SMILES: CC(C)N(CCC1CNc2cccc(F)c21)C(C)C] |
| (S)-I-133 | 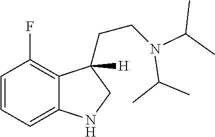 [SMILES: CC(C)N(CC[C@@H]1CNc2cccc(F)c21)C(C)C] |
| (R)-I-133 | 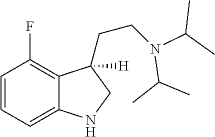 [SMILES: CC(C)N(CC[C@H]1CNc2cccc(F)c21)C(C)C] |
| I-134 | 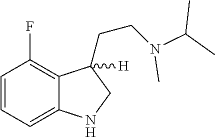 [SMILES: CC(C)N(C)CCC1CNc2cccc(F)c21] |
| (R)-I-134 | 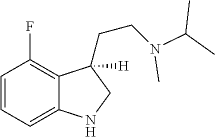 [SMILES: CC(C)N(C)CC[C@H]1CNc2cccc(F)c21] |
| (S)-I-134 | 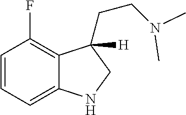 |
| I-135 | 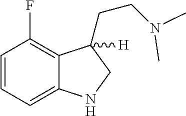 |
| (R)-I-135 | 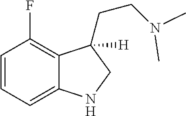 |
| (S)-I-135 | 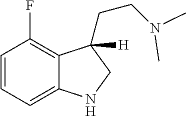 |
| I-136 | 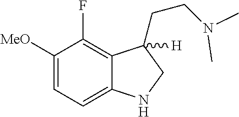 [SMILES: COc1ccc2c(c1F)C(CCN(C)C)CN2] |
| (S)-I-136 | 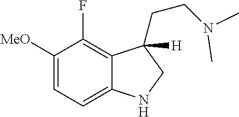 [SMILES: COc1ccc2c(c1F)[C@H](CCN(C)C)CN2] |
| (R)-I-136 | 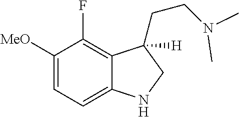 [SMILES: COc1ccc2c(c1F)[C@@H](CCN(C)C)CN2] |
| I-137 | 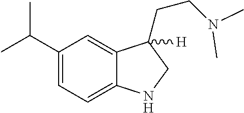 [SMILES: CC(C)c1ccc2c(c1)C(CCN(C)C)CN2] |
| (R)-I-137 | 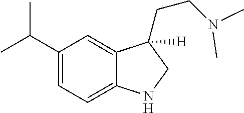 [SMILES: CC(C)c1ccc2c(c1)[C@@H](CCN(C)C)CN2] |
| (S)-I-137 | 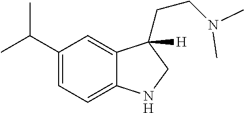 [SMILES: CC(C)c1ccc2c(c1)[C@H](CCN(C)C)CN2] |
| I-138 | 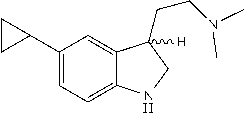 [SMILES: CN(C)CCC1CNc2ccc(C3CC3)cc21] |
| I-139 | 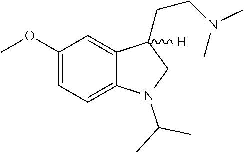 |
| I-140 | 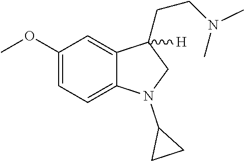 |
| I-141 | 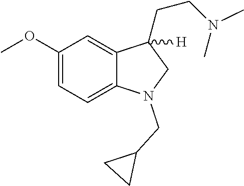 |
| I-142 | 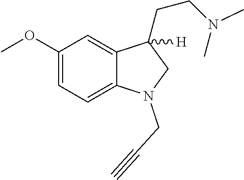 |
| I-143 | 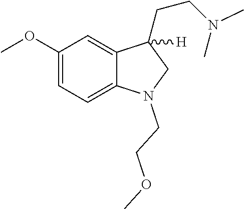 |
| I-144 | 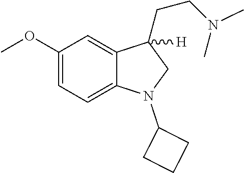 |
| I-145 | 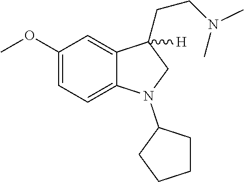 |
| I-146 | 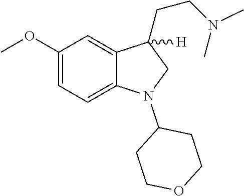 |
| I-147 | 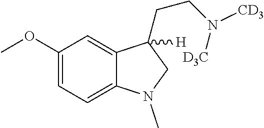 [SMILES: [2H]C([2H])([2H])N(CCC1CN(C)c2ccc(OC)cc21)C([2H])([2H])[2H]] |
| (R)-I-147 | 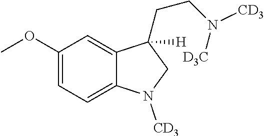 [SMILES: [2H]C([2H])([2H])N1C[C@H](CCN(C([2H])([2H])[2H])C([2H])([2H])[2H])c2cc(OC)ccc21] |
| (S)-I-147 | 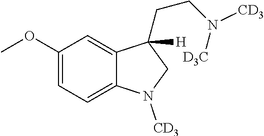 [SMILES: [2H]C([2H])([2H])N1C[C@@H](CCN(C([2H])([2H])[2H])C([2H])([2H])[2H])c2cc(OC)ccc21] |

or a pharmaceutically acceptable salt, solvate, and/or prodrug thereof.

60. (canceled)

61. A pharmaceutical composition comprising a compound of Formula I or a pharmaceutically acceptable salt, solvate, and/or prodrug thereof, and pharmaceutically acceptable carrier, wherein Formula I is: 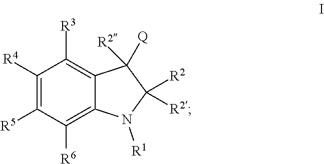
and
wherein:
R¹ is selected from C(O)R⁷, CO₂R⁷, C(O)N(R⁷)(R^{7′}), S(O)R⁷, SO₂R⁷, C₁₋₆alkyleneR⁷, and R⁷;
Q is Q3: 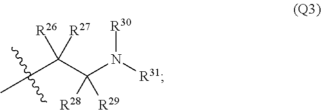 [symbol]
over a bond means that the bond is attached to a remaining portion of the compound;
R², R^{2′}, and R^{2″} are independently selected from H, halo, C₁₋₆alkyl, C₁₋₆alkoxy, C₁₋₆alkyleneN(R^{2a})(R^{2b}), and SC₁₋₆alkyl;
R^{2a} and R^{2b} are independently selected from H and C₁₋₆alkyl;
R³, R⁴, R⁵, and R⁶ are independently selected from H, halo, N(R^{5′})(R^{6′}), C₁₋₆alkyl, C₁₋₆alkoxy, C₁₋₆ alkyleneN(R^{5′})(R^{6′}), and SC₁₋₆alkyl, the latter four groups being optionally substituted with one or two substituents independently selected from OH and C₁₋₆alkoxy, or
two adjacent R³, R⁴, R⁵, and R⁶ are linked together to form O—(CH₂)₁₋₂O and the remaining of R³, R⁴, R⁵, and R⁶ are independently selected from H, halo, C₁₋₆alkyl, C₁₋₆alkoxy, N(R^{5′})(R^{6′}), C₁₋₆alkyleneN(R^{5′})(R⁶′), and SC₁₋₆alkyl, or
one of R³, R⁴, R⁵, and R⁶ is selected from A, O-A, and C₁₋₄alkyleneA and the remaining of R³, R⁴, R⁵, and R⁶ are independently H or halo;
R^{5′} and R^{6′} are independently selected from H and C₁₋₆alkyl;
A is selected from phenyl, C₃₋₆cycloalkyl, 3- to 6-membered heterocycloalkyl comprising 1 to 4 heteromoieties independently selected from O, S, S(O), SO₂, N, and NR⁵⁴ and 5- to 6-membered heteroaryl comprising 1 to 4 heteromoieties independently selected from O, S, S(O), SO₂, N, and NR⁵⁴, wherein the phenyl, C₃₋₁₀cycloalkyl, 3- to 6-membered heterocycloalkyl, and 5- to 6-membered heteroaryl are optionally substituted with one or more substituents independently selected from halo, C₁₋₄alkyl, OC₁₋₄alkyl, N(R^{54a})(R^{54b}), C₁₋₄alkyleneN(R^{54a})(R^{54b}), and SC₁₋₄alkyl;
R⁷ is selected from H, C₁₋₆alkyl, C₂₋₆alkenyl, C₂₋₆alkynyl, phenyl, C₃₋₆cycloalkyl, 3- to 6-membered heterocycloalkyl comprising 1 to 4 heteromoieties independently selected from O, S, N, and NR^{7″}, and 5- to 6-membered heteroaryl comprising 1 to 4 heteromoieties independently selected from O, S, N, and NR^{7″}, wherein the latter 7 groups are optionally substituted with one or more substituents independently selected from halo, OR⁵⁵, N(R⁵⁵)(R⁵⁶), and SR⁵⁵ and/or the C₁₋₆alkyl is optionally interrupted by one to three heteromoieties independently selected from O, C(O), CO₂, and NR⁵⁷;
R^{7′} is selected from H and C₁₋₆alkyl;
R^{7″} is selected from H and C₁₋₆alkyl;
R²⁶, R²⁷, R²⁸, and R²⁹ are independently selected from H, halo, and C₁₋₆alkyl;
R³⁰ and R³¹ are independently selected from H, C₁₋₆alkyl, and C(O)C₁₋₆alkyl, or
R³⁰ and R³¹, together with the N atom to which they are bound, form a 3- to 6-membered heterocyclic ring which optionally comprises one or two additional heteromoieties independently selected from O, S, S(O), SO₂, N, and NR⁵⁸;
R⁵⁴, R⁵⁵, R⁵⁵, R⁵⁷, and R⁵⁸ are independently selected from H and C₁₋₆alkyl;
R^{54a} and R^{54b} are independently selected from H and C₁₋₆alkyl; and
available hydrogen atoms are optionally and independently substituted with a fluorine atom or a chlorine atom and/or available atoms are optionally substituted with alternate isotope thereof,
provided that when R²⁶, R²⁷, R²⁸, and R²⁹ are all H and R³⁰ and R³¹ are H or CH₃, and:
R¹, R², R^{2′}, R^{2″}, R³, R⁴, R⁵, and R⁶ are all H,
R¹, R², R^{2′}, R^{2″}, R³, R⁵, and R⁶ are all H and R⁴ is OCH₃, or
R¹, R², R^{2′}, R^{2″}, R³, R⁴, R⁵, and R⁶ are all H and R⁵ is OCH₃,
then the compound of Formula I is an (R)- or (S)-enantiomer of the carbon to which Q is attached.

62. (canceled)

63. (canceled)

64. (canceled)

65. (canceled)

66. (canceled)

67. (canceled)

68. (canceled)

69. (canceled)

70. A method of treating a central nervous system (CNS) disease, disorder, or condition and/or a neurological disease, disorder, or condition comprising administering a therapeutically effective amount of one or more compounds of Formula I or a pharmaceutically acceptable salt, solvate, and/or prodrug thereof, to a subject in need thereof, wherein Formula I is: 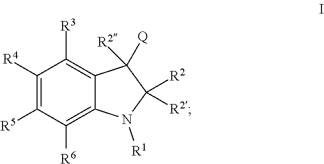
wherein:
R¹ is selected from C(O)R⁷, CO₂R⁷, C(O)N(R⁷)(R^{7′}), S(O)R⁷, SO₂R⁷, C₁₋₆alkyleneR⁷, and R⁷;
Q is Q3: 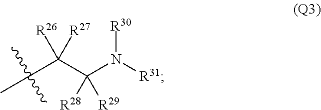 [symbol]
over a bond means that the bond is attached to a remaining portion of the compound;
R², R^{2′}, and R^{2″} are independently selected from H, halo, C₁₋₆alkyl, C₁₋₆alkoxy, C₁₋₆alkyleneN(R^{2a})(R^{2b}), and SC₁₋₆alkyl;
R^{2a} and R^{2b} are independently selected from H and C₁₋₆alkyl;
R³, R⁴, R⁵, and R⁶ are independently selected from H, halo, N(R^{5′})(R^{6′}), C₁₋₆alkyl, C₁₋₆alkoxy, C₁₋₆ alkyleneN(R^{5′})(R^{6′}), and SC₁₋₆alkyl, the latter four groups being optionally substituted with one or two substituents independently selected from OH and C₁₋₆alkoxy, or
two adjacent R³, R⁴, R⁵, and R⁶ are linked together to form O—(CH₂)₁₋₂O and the remaining of R³, R⁴, R⁵, and R⁶ are independently selected from H, halo, C₁₋₆alkyl, C₁₋₆alkoxy, N(R^{5′})(R^{6′}), C₁₋₆alkyleneN(R^{5′})(R⁶′), and SC₁₋₆alkyl, or
one of R³, R⁴, R⁵, and R⁶ is selected from A, O-A, and C₁₋₄alkyleneA and the remaining of R³, R⁴, R⁵, and R⁶ are independently H or halo;
R^{5′} and R^{6′} are independently selected from H and C₁₋₆alkyl;
A is selected from phenyl, C₃₋₆cycloalkyl, 3- to 6-membered heterocycloalkyl comprising 1 to 4 heteromoieties independently selected from O, S, S(O), SO₂, N, and NR⁵⁴ and 5- to 6-membered heteroaryl comprising 1 to 4 heteromoieties independently selected from O, S, S(O), SO₂, N, and NR⁵⁴, wherein the phenyl, C₃₋₁₀cycloalkyl, 3- to 6-membered heterocycloalkyl, and 5- to 6-membered heteroaryl are optionally substituted with one or more substituents independently selected from halo, C₁₋₄alkyl, OC₁₋₄alkyl, N(R^{54a})(R^{54b}), C₁₋₄alkyleneN(R^{54a})(R^{54b}), and SC₁₋₄alkyl;
R⁷ is selected from H, C₁₋₆alkyl, C₂₋₆alkenyl, C₂₋₆alkynyl, phenyl, C₃₋₆cycloalkyl, 3- to 6-membered heterocycloalkyl comprising 1 to 4 heteromoieties independently selected from O, S, N, and NR^{7″}, and 5- to 6-membered heteroaryl comprising 1 to 4 heteromoieties independently selected from O, S, N, and NR^{7″}, wherein the latter 7 groups are optionally substituted with one or more substituents
independently selected from halo, OR⁵⁵, N(R⁵⁵)(R⁵6), and SR⁵⁵ and/or the C₁₋₆alkyl is optionally interrupted by one to three heteromoieties independently selected from O, C(O), CO₂, and NR⁵⁷;
R^{7′} is selected from H and C₁₋₆alkyl;
R^{7″} is selected from H and C₁₋₆alkyl;
R²⁶, R²⁷, R²⁸, and R²⁹ are independently selected from H, halo, and C₁₋₆alkyl;
R³⁰ and R³1 are independently selected from H, C₁₋₆alkyl, and C(O)C₁₋₆alkyl, or
R³⁰ and R³¹, together with the N atom to which they are bound, form a 3- to 6-membered heterocyclic ring which optionally comprises one or two additional heteromoieties independently selected from O, S, S(O), SO₂, N, and NR⁵⁸;
R⁵⁴, R⁵⁵, R⁵⁵, R⁵⁷, and R⁵⁸ are independently selected from H and C₁₋₆alkyl;
R^{54a} and R^{54b} are independently selected from H and C₁₋₆alkyl; and
available hydrogen atoms are optionally and independently substituted with a fluorine atom or a chlorine atom and/or available atoms are optionally substituted with alternate isotope thereof, provided that when R²⁶, R²⁷, R²⁸, and R²⁹ are all H and R³⁰ and R³¹ are H or CH₃, and:
R¹, R², R^{2′}, R^{2″}, R³, R⁴, R⁵, and R⁶ are all H,
R¹, R², R^{2′}, R^{2″}, R³, R⁵, and R⁶ are all H and R⁴ is OCH₃, or
R¹, R², R^{2′}, R^{2″}, R³, R⁴, R⁵, and R⁶ are all H and R⁵ is OCH₃,
then the compound of Formula I is an (R)- or (S)-enantiomer of the carbon to which Q is attached.

71. (canceled)

72. (canceled)

73. (canceled)

74. (canceled)

75. (canceled)

76. (canceled)

77. (canceled)

78. (canceled)

79. (canceled)

80. (canceled)

81. (canceled)
